(19) United States
(12) Patent Application Publication
(10) Pub. No.: US 2025/0051768 A1
(43) Pub. Date: Feb. 13, 2025

(54) NON-CODING RNA-MEDIATED NEUROLOGICAL DISEASE TREATMENT

(71) Applicant: SHANGHAI GENEMAGIC BIOSCIENCES CO., LTD., Shanghai (CN)

(72) Inventors: Haibo ZHOU, Shanghai (CN); Xinde HU, Shanghai (CN); Jinlin SU, Shanghai (CN)

(73) Assignee: SHANGHAI GENEMAGIC BIOSCIENCES CO., LTD., Shanghai (CN)

(21) Appl. No.: 18/717,671

(22) PCT Filed: Dec. 6, 2022
(86) PCT No.: PCT/CN2022/136909

§ 371 Date: Jun. 7, 2024

(30) Foreign Application Priority Data
Dec. 7, 2021 (CN) 202111488197.4

(51) Int. Cl. C12N 15/113; A61K 31/7105; A61K 31/711; C07K 16/44; C12N 15/86

(52) U.S. Cl. CPC C12N 15/113; A61K 31/7105; A61K 31/711; C07K 16/44; C12N 15/86; C12N 2310/141; C12N 2750/14143

(57) ABSTRACT

A method for producing neurons from non-neuronal cells is provided, and comprises: by means of enhancing or reducing the expression or activity of certain miRNAs and/or lncRNAs in non-neuronal cells, trans differentiating or reprogramming the non-neuronal cells into neurons. Also provided is the use of reagents that enhance or reduce the expression or activity of certain miRNAs and/or lncRNAs in the prevention and/or treatment of diseases related to neuronal dysfunction or death.

This application claims the priority of the application with the title of “non-coding RNA-mediated treatment of neurological disease treatment” which application number is 202111488197.4, the priority application was submitted on Dec. 7, 2021, the entire contents of which are incorporated herein by reference.

### TECHNICAL FIELD

The present disclosure relates to the field of biomedicine. More specifically, the present disclosure relates to the use of reagents that increase or decrease the expression or activity of certain non-coding RNAs, such as certain miRNAs and lncRNAs, for the treatment of diseases associated with neuronal function loss or neuronal death.

### BACKGROUND

Cell transdifferentiation refers to the process in which one type of differentiated cell transforms into another differentiated cell in structure and function through gene selective expression or gene reprogramming.

MicroRNA (miRNA) is a kind of highly conserved non-coding functional RNA molecule existing in animal or plant cell with a length of approximately 17-27 nucleotides. miRNA causes translation inhibition or RNA degradation by binding to the 3′UTR region of the target mRNA sequence, thus inhibiting the function of the target gene. When a miRNA is fully complementary to the target RNA, it leads to the degradation of the mRNA, while when it is not fully complementary to the target RNA, it prevents gene translation. In mammalian cells, miRNAs mainly prevent mRNA translation rather than affect its stability. Long non-coding RNA (LncRNA) is a class of RNA molecules with a length of more than 200 bases, which does not participate in protein coding, but can participate in the regulation of gene expression in the form of RNA. In the past, LncRNA was regarded as the “noise” of gene transcription, a by-product of RNA transcription without biological function. However, studies in recent years have shown that lncRNAs extensively participates in various biological processes, such as gene transcriptional regulation, genomic imprinting, chromatin silencing, chromatin modification, activation or inhibition of gene expression, and many other biological processes. About 4-8% of the sequences in the genome is used to transcribe LncRNA, but the functional research of LncRNA is rather limited. Furthermore, due to technological constraints, the research of LncRNA is relatively lagging behind. LncRNAs participate in the regulation of gene expression from multiple levels such as epigenetics, gene transcription regulation, and post-transcriptional regulation, and some LncRNAs also participate in the regulation of miRNAs, thereby indirectly regulating gene expression.

In the central nervous system, astrocytes are a very abundant type of glial cells that exist around neurons and support and provide nutrition to neurons. In a variety of neurodegenerative diseases or neurotrauma, neuronal cells will die, resulting in some functional deficits, such as stroke, Alzheimer's disease and Parkinson's disease. Scientists can transdifferentiate glial cells into neurons by adding a variety of factors to astrocytes cultured in vitro, such as ASCL1, NeuroD1 and Ngn2. But in vivo, due to the complexity of the environment in the body and the interaction between cells, it is difficult to reproduce the results of many studies in vitro culture dishes. Therefore, it has great scientific significance to study how to transdifferentiate astrocytes in situ in vivo to replenish lost neurons.

Parkinson's disease (PD) is a disorder associated with neuronal function loss or neuronal death which is characterized by the loss of dopaminergic neurons in the substantia nigra of the midbrain. At present, the main treatment method for Parkinson's disease is the medicine represented by levodopa formulation. Meanwhile, surgical treatment can also improve symptoms to a certain extent. It should be pointed out that all these methods can only partially alleviate the disease, and are not yet effective in preventing its progression.

In recent years, the exploration of the functions of miRNAs and LncRNAs in the nervous system has gradually increased, which mainly focus on the distribution of different miRNAs and LncRNAs in the nervous system and the functions of miRNAs and LncRNAs in the growth and development of the nervous system. There are fewer studies in neurological diseases. Among them, the functions of miR-9 and miR-124 in neuronal development and maturation are mostly studied. There are very few studies in the use of the characteristics of miRNA and LncRNA to regulate the gene expression network in order to change the characteristics of cells. miRNA and LncRNA have very important functions. It has great scientific significance to explore how to apply miRNA and LncRNA to develop drugs, more specifically, nerve regeneration drugs.

There is still an urgent need in the field to develop new targets and new therapies that can effectively treat diseases associated with neuronal function loss or neuronal death.

### Contents of the Invention

In one embodiment, the present disclosure provides a method for producing neuronal cells from non-neuronal cells, the method comprise transdifferentiation or reprogramming the non-neuronal cells into neuronal cells by enhancing the expression or activity of miRNA selected from Let-7a, Let-7b, miR-18a/b, miR-24-3p, miR-34a, miR-92b, miR-96, miR-106, miR-125a/b, miR-128, miR-134, miR-135, miR-137, miR-141, miR-143-3p, miR-184, miR-200, miR-218, miR-219, miR-228, miR-28, miR-429, miR-430, or lncRNA selected from utNgn1, RMST, Tuna, Linc-Brn1b, Dali, Miat/Gomafu, NBAT-1, Malat1, Dlx1as, Six3os, Evf2, LncKdm2b, lncRNA_N1, lncRNA_N2, lncRNA_N3, or any combination thereof.

In a preferred embodiment, the expression or activity of the miRNA or lncRNA is enhanced through, for example, overexpression, gene activators, epigenetic modifications, miRNA mimics, direct delivery of RNA, small-molecule compounds, and/or RNA stabilizers.

In another embodiment, the present disclosure provides a method for producing neuronal cells from non-neuronal cells, which comprise the transdifferentiation or reprogramming the non-neuronal cells into neuronal cells by reducing the expression or activity of miRNA selected from miR-7a, miR-15, miR-23a/b, miR-25, miR-29a, miR-129, miR-137, miR-138, miR-155, miR-195, miR-214, miR-222, miR-223, miR-132, miR-133, or lncRNA selected from Pnky, Paupar, HOTAIRM1, IncR492, TUG1, or any combination thereof.

In a preferred embodiment, the expression or activity of the miRNA or lncRNA is reduced by techniques such as DNA editing or RNA editing induced by gene editing technologies, RNA expression inhibitors, antisense oligonucleotides (ASO), small RNA interference, miRNA technology, small-molecule compounds, gene inhibiting techniques (e.g., dCas-Krab), and/or epigenetic regulation.

In a preferred embodiment, RNA editing includes CRISPR-mediated RNA degradation or translation inhibition, RNA single base editing, insertion or deletion of bases of RNA, alteration of RNA splicing, or RNA epigenetic modification.

In a preferred embodiment, the miRNA selected from Let-7a, Let-7b, miR-18a/b, miR-24-3p, miR-34a, miR-92b, miR-96, miR-106, miR-125a/b, miR-128, miR-134, miR-135, miR-137, miR-141, miR-143-3p, miR-184, miR-200, miR-218, miR-219, miR-228, miR-284, miR-429, miR-430, or the miRNA selected from miR-7a, miR-15, miR-23a/b, miR-25, miR-29a, miR-129, miR-137, miR-138, miR-155, miR-195, miR-214, miR-222, miR-223, miR-132, miR-133, or the lncRNA selected from utNgn1, RMST, Tuna, Linc-Brn1b, Dali, Miat/Gomafu, NBAT-1, Malat1, Dlx1as, Six3os, Evf2, LncKdm2b, lncRNA_N1, lncRNA_N2, lncRNA_N3, or the lncRNA selected from Pnky, Paupar, HOTAIRM1, IncR492, TUG1 are homologous miRNA or homologous lncRNA from different species.

In a preferred embodiment, the non-neuronal cells comprise, for example, glial cells, fibroblasts, stem cells, neural precursor cells, neural stem cells, wherein the glial cells are selected from astrocytes, microglia, oligodendrocytes, ependymal cells, Schwann cells, NG2 cells, satellite cells, or any combinations thereof, preferably are astrocytes.

In a preferred embodiment, the glial cells are derived from the brain, spinal cord, eyes or ears, wherein the glial cells in the brain are derived from the striatum, substantia nigra, ventral tegmental area of the midbrain, spinal cord, hypothalamus, dorsal midbrain, or cerebral cortex, preferably, the glial cells in the brain are derived from striatum and substantia nigra.

In a preferred embodiment, the neuronal cells are preferably selected from dopaminergic neurons, GABA neurons, 5-HT neurons, glutamatergic neurons, ChAT neurons, NE neurons, motor neurons, spinal cord neurons, spinal motor neurons, spinal sensory neurons, pyramidal neurons, intemeurons, medium spiny neurons (MSN), Purkinje cells, granule cells, olfactory sensory neurons, periglomerular cells, or any combinations thereof, more preferably are dopaminergic neurons.

In a preferred embodiment, said non-neuronal and/or neuronal cells are derived from, for example, humans, non-human primates, rats and mice, preferably from humans.

In a preferred embodiment, the method is an in vivo method or an in vitro method.

In another embodiment, the disclosure provides the use of the reagent that enhances the expression or activity of miRNA selected from Let-7a, Let-7b, miR-18a/b, miR-24-3p, miR-34a, miR-92b, miR-96, miR-106, miR-125a/b, miR-128, miR-134, miR-135, miR-137, miR-141, miR-143-3p, miR-184, miR-200, miR-218, miR-219, miR-228, miR-284, miR-429, miR-430, or lncRNA selected from utNgn1, RMST, Tuna, Linc-Brn1b, Dali, Miat/Gomafu, NBAT-1, Malat1, Dlx1as, Six3os, Evf2, LncKdm2b, lncRNA_N1, lncRNA_N2, lncRNA_N3, or any combination thereof, which is used to prepare the drugs for preventing and/or treating diseases associated with neuronal function loss or neuronal death, wherein the reagent enhances the expression or activity of the miRNA or lncRNA.

In another embodiment, the disclosure provides the use of the reagent that reduces the expression or activity of miRNA selected from miR-7a, miR-15, miR-23a/b, miR-25, miR-29a, miR-129, miR-137, miR-138, miR-155, miR-195, miR-214, miR-222, miR-223, miR-132, miR-133, or lncRNA selected from Pnky, Paupar, HOTAIRM1, IncR492, TUG1, or any combination thereof, in the preparation of drugs for preventing and/or treating diseases associated with neuronal function loss or neuronal death, wherein the reagent reduces the expression or activity of the miRNA or lncRNA.

In a preferred embodiment, the drug is formulated for in vivo administration to the nervous system, such as striatum, substantia nigra, ventral tegmental area of midbrain, spinal cord, hypothalamus, dorsal midbrain, cerebral cortex, hippocampus, cerebellum, inner ear cochlea or vestibular system, preferably formulated for administration to striatum, substantia nigra, subretinal space, vitreous cavity or inner ear cochlea.

In a preferred embodiment, the disease associated with neuronal function loss or neuronal death is selected from: Parkinson's disease, Alzheimer's disease, stroke, schizophrenia, Huntington's disease, depression, motor neuron disease, cerebral ischemia, brain injury, amyotrophic lateral sclerosis (ALS), spinal muscular atrophy, Pick's disease, sleep disorders, epilepsy, ataxia, PloyQ disease, addiction, or a combination thereof, preferably is Parkinson's disease.

In a preferred embodiment, said enhancing the expression or activity of said miRNA or lncRNA or a combination thereof comprises:
(a) exogenously expressing the miRNA or lncRNA or a combination thereof, for example, exogenously expressing was achieved by an expression vector comprising a promoter;
(b) delivering the miRNA or lncRNA or a combination thereof in the form of DNA or RNA into the cell;
(c) activating the endogenous expression of the miRNA or lncRNA or a combination thereof, such as gene expression activators and epigenetic regulatory elements, etc.; or
(d) delivering an analog or agonist of said miRNA or lncRNA or a combination thereof to the cell;
wherein, it is preferable to express the miRNA or lncRNA or a combination thereof exogenously, for example, through an expression vector comprising a promoter.

In a preferred embodiment, the expression or activity of said miRNA or lncRNA or a combination thereof is reduced through the use of: antibody, small molecule compound, microRNA, siRNA, shRNA, antisense oligonucleotide, binding protein or protein domain, polypeptides, nucleic acid aptamers, gene editors, epigenetic regulatory elements, transcriptional repression elements, or any combinations thereof.

In another embodiment, the present disclosure provides a pharmaceutical composition or pharmaceutical kit or reagent kit comprising a reagent that enhance the expression or activity of miRNA selected from Let-7a, Let-7b, miR-18a/b, miR-24-3p, miR-34a, miR-92b, miR-96, miR-106, miR-125a/b, miR-128, miR-134, miR-135, miR-137, miR-141, miR-143-3p, miR-184, miR-200, miR-218, miR-219, miR-228, miR-284, miR-429, miR-430, or lncRNA selected from utNgn1, RMST, Tuna, Linc-Bm1b, Dali, Miat/Gomafu, NBAT-1 Malat1, Dlx1as, Six3os, Evf2, LncKdm2b, lncRNA_N1, lncRNA_N2, lncRNA_N3, or any combination thereof; or a reagent that reduce the expression or activity of miRNA selected from miR-7a, miR-15, miR-23a/b, miR-25, miR-29a, miR-129, miR-137, miR-138, miR-155, miR-195, miR-214, miR-222, miR-223, miR-132, miR-133, or lncRNA selected from Pnky, Paupar, HOTAIRM1, IncR492, TUG1, or any combination thereof.

In a preferred embodiment, the reagent for enhancing the expression or activity of the miRNA or lncRNA or a combination thereof is selected from: an expression vector, the miRNA or lncRNA or a combination thereof in the form of DNA or RNA, a endogenous activator of the miRNA or lncRNA or a combination thereof, an analog or agonist of said miRNA or lncRNA or combinations thereof.

In a preferred embodiment, the expression vector is a gene therapy vector, preferably is a viral gene therapy vector, more preferably the viral vector is selected from: adeno-associated virus (AAV) vector, recombinant adeno-associated virus vector (rAAV), self-complementary AAV (scAAV) vector, adenovirus vector, lentivirus vector, retrovirus vector, herpesvirus, SV40 vectors, poxvirus vectors, and any combination thereof, wherein the viral vector preferably is AAV or rAAV.

In a preferred embodiment, the reagent that reduces the expression or activity of the miRNA or lncRNA or a combination thereof, is selected from the group consisting of antibodies, small molecule compounds, microRNA, siRNA, shRNA, antisense oligonucleotides, binding proteins or proteins domains, polypeptides, aptamers, gene editors, epigenetic regulatory elements, transcriptional repressor elements, or combinations thereof.

In a preferred embodiment, said pharmaceutical composition or pharmaceutical kit or reagent kit further comprises a carrier or vehicle for delivering said reagent.

In a preferred embodiment, the carrier or vehicle is a viral vector, liposome, nanoparticle, exosome, virus-like particle, preferably is AAV.

In a preferred embodiment, the composition is locally administered to at least one of the following: i) glial cells in the striatum; ii) glial cells in the ventral tegmental area (VTA); iii) glial cells in the substantia nigra. iv) glial cells in the hypothalamus; v) glial cells in the spinal cord; vi) glial cells in the prefrontal cortex; and vii) glial cells in the motor cortex.

In a preferred embodiment, the pharmaceutical composition or pharmaceutical kit or reagent kit is formulated for cell transfection, cell infection, endocytosis, injection, intracranial administration, inhalation, parenteral administration, intravenous administration, intramuscular administration, intradermal administration, epidermal administration, or oral administration.

In a preferred embodiment, wherein said miRNA or lncRNA is Let-7a, miR-92b, miR-96, miR-106, miR-125a, miR-135, miR-141, miR-200, miR-218, miR-429, or miR-24, the non-neuronal cells are glial cells, and the neuronal cells are dopaminergic neurons.

In a preferred embodiment, the transdifferentiation efficiency of non-neuronal cells is at least 1%, or at least 10%, 20%, 30%, 40%, or 50%.

the various technical features described above and those specifically described in the following (such as in the embodiments) can be combined with each other to form new or preferred technical solutions. Due to the limited space, they will not be enumerated here one by one.

### FIGURES

FIG. 1 Transdifferentiation of glial cell to neuron induced by Pnky knocking down. (A) Schematic diagram of AAV vector design. Vector 1 is a vector schematic diagram of GFAP-driven mCherry expression, GFAP is a promoter specifically expressed in glial cells, mCherry is a red fluorescent protein which is used to label glial cells. Vector 2 is a schematic diagram of the GFAP-CasRx expression vector, the expression of CasRx is driven by the astrocyte-specific promoter GFAP. Vector3 is a schematic diagram of the U6-gRNA-GFAP-CasRx expression vector. The expression of gRNA is promoted by the U6 promoter, and the expression of CasRx is promoted by the GFAP promoter. (B) Using CasRx to knock down the expression of Pnky in 293T cells, CasRx-gRNA (Pnky) efficiently knocked down the mRNA of Pnky. (C) Schematic diagram of AAV injection and transdifferentiation. Different AAVs were injected into the mouse brain to study the transdifferentiation of glial cells into neurons: the control group was injected with a mixed virus of GFAP-mCherry and GFAP-CasRx, and the experimental group was injected with a mixed AAV of GFAP-mCherry and GFAP-CasRx-gRNA (Pnky), GFAP-mCherry labels astrocytes.

FIG. 2 Transdifferentiation of glial cell to neuron induced by miRNA or LncRNA. (A) Schematic diagram of the design of GFAP-mCherry and GFAP-miRNA/lncRNA expression vectors. Expression of mCherry, miRNA or LncRNA are driven by the glial cell-specific promoter GFAP. (B) Schematic diagram of AAV expression vectors for Tuna, Let-7b, and miR-137, wherein Tuna is LncRNA, and Let-7b and miRNA-137 are miRNAs, both of which are driven to express by the GFAP promoter. (C) Schematic diagram of AAV injection and transdifferentiation from glial cells into neurons by miRNA/LncRNA. (D) Representative diagram of the results of AAV injection into the striatum of C57 mice in the control group. The samples were analyzed one month after AAV injection. The red fluorescent signal represented the cells labeled by GFAP-mCherry, and the white signal represented the neuron-specific marker NeuN. There was no overlap between the white and red signals. (E-G) Representative diagram of the results which analyzed one month after injection of GFAP-Tuna, GFAP-Let-7b or miR-137 in the striatum of mice, the red fluorescent signal (mCherry) represented the neurons labeled by GFAP-mCherry, The white signal represented the neuron-specific marker NeuN, the yellow arrow indicated the cells co-labeled with mCherry and NeuN, and the scale bar was 50 μm.

FIG. 3 Transdifferentiation of astrocytes into neurons induced by the overexpression of miRNAs. (A) Schematic diagram of AAV vector, the vector 1 was GFAP-EGFP-labeled AAV, which specifically labeled astrocytes, and the vector 2 was GFAP-activated miRNA or LncRNA, which specifically expressed miRNA or LncRNA in glial cells. (B) The control group was injected with AAV-GFAP-EGFP, EGFP specifically labeled astrocytes. (C-G) The representive figures of transdifferentiation of astrocytes into neurons after the injection of AAV (GFAP-miR-18b, GFAP-miR-34a, GFAP-miR-128, GFAP-miR-134, GFAP-miR-143) into the striatum of the mouse. The green fluorescent signal represented AAV-GFAP-EGFP-labeled cells, and NeuN represented a neuron-specific protein marker. White arrows pointed to the cells co-labeled with green and white fluorescence, which showed that these cells expressed the neuron-specific protein marker NeuN, and the bar was 50 μm.

FIG. 4 Overexpression of miRNAs induces transdifferentiation of astrocytes into dopaminergic neurons. (A) Analysis was conducted on samples obtained one month after injection of the control group's AAV-GFAP-EGFP into the striatum of mice. (B-F) Overexpression of miRNAs (Let-7a, miR-92b, miR-96, miR-24, miR-106, -miR-125a) in the astrocytes of the mouse striatum enabled the transdifferentiation of astrocytes into neurons, among which some glial cells are transdifferentiated into dopaminergic neurons. The white arrow pointed to the green cells labeled with GFAP-EGFP, the white signal represented the neuron-specific marker NeuN, the red signal represented the dopamine-specific protein marker TH, and the merged figures showed the partial overlap between the different signals. White arrows pointed to the cells which were co-labeled with the green signal and NeuN, while the yellow arrows pointed to TH-positive cells, and the bar was 50 μm.

FIG. 5 Overexpression of miRNAs transdifferentiates astrocytes into dopaminergic neurons. (A-F) Overexpression of miR-135, miR-24, miR-141, miR-200, miR-218, miR-429 ect. in the astrocytes of the mouse striatum enabled the transdifferentiation of astrocytes into neurons, with some glial cells transdifferentiating into dopaminergic neurons. The white arrow pointed to the green cells labeled with GFAP-EGFP, the white signal represented the neuron-specific marker NeuN, the red signal represented the dopamine-specific protein marker TH, and the merged figures showed the overlap between different signals. White arrows pointed to cells which were co-labeled with the green signal and NeuN, while the yellow arrows pointed to TH-positive cells, and the bar was 50 μm.

FIG. 6 Proportions of astrocyte transdifferentiation into neurons and dopaminergic neurons via overexpression of miRNAs. (A) The efficiency of astrocyte transdifferentiation into neurons was analyzed after overexpressing miRNA such asmiR-18b, miR-24, miR-34a, miR-128, miR-134, miR-143, Let-7a, miR-92b miR-96, miR-106, miR-125a, miR-135, miR-141, miR-200, miR-218, or miR-429 in the astrocytes of mouse striatum. (B) The efficiency of astrocyte transdifferentiation into dopaminergic neurons was statistically analyzed after overexpressing Let-7a, miR-106, miR-125, miR-134, miR-135, miR-141, miR-200, miR-429 in the astrocytes of mouse striatum.

### DETAILS

miRNA and LncRNA are widely expressed in the nervous system and have important functions, and play an important role in the development of the nervous system as well as the occurrence and development of many neurological diseases. During the development of the nervous system, miRNAs are widely expressed in a variety of nerve cells, and different neural cells express different types of miRNAs. This indicates that different miRNAs may be involved in regulating the development of different neural cells. Previous studies have shown that miRNA-124 can promote the differentiation of neural precursor cells into neurons during the development of the nervous system, and miRNA-124 can inhibit the differentiation of neural precursor cells into glial cells by regulating the expression of genes such as Ptbpl. Moreover, overexpression of miRNA-9 and miRNA-124 can transdifferentiate Müller glia cells into retinal neurons when Müller glia cells MG) cultured in vitro, and the overexpression of miRNA-9 and miRNA-124 can promote AscL1-mediated transdifferentiation of Müller glial cells into retinal neurons. LncRNA is a kind of non-coding functional RNA that is very abundant in the nervous system, and about half of the lncRNA is specifically expressed in the nervous system. LncRNAs not only participates in the development and functional maturation of the nervous system, but also participate in the regulation of synaptic connections, axon growth, and post-injury repair of the nervous system. However, due to limitations in technological methods, there are fewer studies on LncRNA, and the functions of many LncRNAs are still unclear. Previous studies have shown that lncRNAs have important functions in regulating brain development, such as promoting the differentiation of neural stem cells into neurons or glial cells. LncRNA also has important functions in the process of disease injury repair. Studies have found that BACE1-AS is related to Alzheimer's disease, and MALAT1 is related to the occurrence and development of Parkinson's disease. However, there is no research on the role of lncRNA in glial cell transdifferentiation currently.

With the development of the aging, neurodegenerative diseases have gradually become the second largest disease after cancer, affecting a large number of patients. However, there are very few drugs that can be used to treat neurodegenerative diseases, and most of them can only delay the progression of the disease, rather than reversing it. The emergence of nerve regeneration technology undoubtedly brings hope for the treatment of these significant neurodegenerative diseases. Scientists transdifferentiate glial cells into functional neurons in hope of the treating the serious brain diseases such as neurodegenerative diseases or stroke. Therefore, it is particularly important to find high-efficiency neural transdifferentiation targets.

In this study, glial cells were transdifferentiated into neurons in the nervous system by AAV-mediated gene delivery technology. The experimental results showed that miRNAs or LncRNAs such as Pnky, Tuna, Let-7b and miR137 could transdifferentiate astrocytes into neurons in vivo. These results indicate that miRNAs and LncRNAs also can serve as targets for the transdifferentiation of glial cells into neurons, and lay the foundation for the treatment of various neurodegenerative diseases.

### Diseases Associated with Neuronal Function Loss or Neuronal Death

In the present disclosure, diseases related to neuronal function loss or neuronal death mainly include diseases related to dopaminergic neurons function loss or death and visual impairment related to retinal ganglion cell or photoreceptor cells loss or death.

In a preferred embodiment, the diseases associated with neuronal function loss or neuronal death are selected from: Parkinson's disease, Alzheimer's disease, stroke, schizophrenia, Huntington's disease, depression, motor neuron disease, cerebral ischemia, brain injury, amyotrophic lateral sclerosis (ALS), spinal muscular atrophy, Pick's disease, sleep disorders, epilepsy, ataxia, PloyQ disease, addiction, or a combination thereof, preferably, the diseases is Parkinson's disease or disorders of the visual system resulting from RGCs or photoreceptor cells function loss or death.

### Astrocytes

Astrocytes are the most numerous cells in the mammalian brain. They perform many functions, including biochemical support (such as forming the blood-brain barrier), providing nutrition to neurons, maintaining extracellular ion balance, and participating in the repair and scar formation after brain and spinal cord injury. Astrocytes can be divided into two types according to the content of glial filaments and the shape of neurite: fibrous astrocytes, which are mostly distributed in the white matter of the brain and spinal cord, have slender processes and fewer branches, and contain a large number of glial filaments in the cytoplasm; protoplasmic astrocytes, which are mostly distributed in the gray matter, have thick and short cell processes and many branches.

The astrocytes that can be used in the present disclosure are not particularly limited, and include various astrocytes derived from the central nervous system of mammals, such as striatum, ventral tegmental area of midbrain, hypothalamus, spinal cord, dorsal midbrain or cerebral cortex, preferably derived from striatum or substantia nigra.

### Neurons

In the present disclosure, neurons may refer to a neuron capable of transmitting or receiving information through chemical or electrical signals. In some embodiments, neurons exhibit one or more functional properties of mature neurons present in the normal nervous system, including but not limited to: excitability (e.g., the ability to exhibit action potentials, such as rapid rise and subsequent decline) (voltage or membrane potential across the cell membrane), the formation of synaptic connections with other neurons, presynaptic neurotransmitter release, and postsynaptic responses (e.g., excitatory postsynaptic currents or inhibitory postsynaptic current).

In some embodiments, neurons are characterized by the expression of one or more markers of functional neurons, including but not limited to synapsin, synaptophysin, glutamate decarboxylase 67(GAD67), glutamate decarboxylase 65(GAD65), parvalbumin, dopamine- and cAMP-regulated neuronal phosphoprotein 32 (DARPP32), vesicular glutamate transporter 1 (vGLUTT), vesicular glutamate transporter 2 (vGLUT2), acetylcholine, tyrosine hydroxylase(TH), dopamine, vesicular GABA transporter (VGAT) and γ-aminobutyric acid (GABA).

### Dopaminergic Neurons

Dopaminergic neurons contain and release dopamine (DA) as a neurotransmitter. Dopamine belongs to the catecholamine neurotransmitter, which plays an important biological role in the central nervous system. The dopaminergic neurons in the brain are mainly concentrated in the substantia nigra pars compacta (SNc) of the midbrain, ventral tegmental area (VTA), hypothalamus and periventricular. Many experiments have confirmed that dopaminergic neurons are closely related to various diseases of the human body, the most typical disease being Parkinson's disease.

### Adeno-Associated Virus

Due to the small size of Adeno-associated Virus (AAV) compared to other viral vectors, its non-pathogenic nature, and its ability to transduce both dividing and non-dividing cells, gene therapy methods targeting genetic diseases based on AAV vectors have received extensive attention.

Adeno-associated virus (AAV) belongs to the genus Dependovirus of the family Parvoviridae, it is the simplest single-stranded DNA-deficient virus discovered so far. It needs helper virus (usually adenovirus) to participate in replication. It encodes cap and rep genes in two terminal inverted repeats (ITRs). ITRs play a decisive role in virus replication and packaging. The cap gene encodes the viral capsid protein, while the rep gene participates in virus replication and integration. AAV can infect a variety of cells.

Recombinant adeno-associated virus vector (rAAV) is derived from non-pathogenic wild-type adeno-associated virus. Due to its good safety, wide range of host cells (dividing and non-dividing cells), low immunogenicity, and long-term expression of exogenous genes in vivo, it is regarded as one of the most promising gene transfer vectors and has been widely used in gene therapy and vaccine research worldwide. After more than 10 years of research, the biological characteristics of recombinant adeno-associated virus have been deeply understood, especially in terms of its application effects in various cells, tissues and in vivo experiments. In medical research, rAAV has been used in the research of gene therapy for various diseases (including in vivo and in vitro experiments). Meanwhile, as a characteristic gene transfer carrier, it is also widely used in gene function research, disease model construction, and the preparation of gene knockout mice, etc.

In a preferred embodiment of the present disclosure, the vector is a recombinant AAV vector. AAVs are relatively small DNA viruses that can integrate into the genome of the infected cells in a stable and site-specific manner. They are able to infect a large range of the cells without any effect on cell growth, morphology or differentiation, and they do not appear to be involved in human pathology. Each end of the AAV genome contains an inverted terminal repeat (ITR) region of approximately 145 bases, which serves as the origin of replication for the virus. The rest of the genome is divided into two important regions with encapsidation function: the left part of the genome containing the rep gene involved in viral replication and viral gene expression; and the right part of the genome containing the cap gene encoding the viral capsid protein.

AAV vectors can be prepared using standard methods in the field. Any serotype of adeno-associated virus is suitable. Methods for purifying vectors can be found, for example, in U.S. Pat. Nos. 6,566,118, 6,989,264, and 6,995,006, the disclosures of which are incorporated herein by reference in their entireties. The preparation of hybrid vectors is described, for example, in PCT Application No. PCT/US2005/027091, the disclosure of which is incorporated herein by reference in its entirety. The use of AAV-derived vectors for gene transfer in vitro and in vivo has been described (see, e.g., International Patent Application Publication Nos. WO91/18088 and WO93/09239; U.S. Pat. Nos. 4,797,368, 6,596,535, and 5,139,941, and European Patent No. 0488528, each of which is incorporated herein by reference in its entirety). These patent publications describe various AAV-derived constructs in which the rep and/or cap genes are deleted and replaced by the gene of interest, as well as the use of these constructs to transport the gene of interest in vitro (into cultured cells) or in vivo (directly into organisms) Replication-defective recombinant AAV can be prepared by co-transfecting plasmids into cell lines infected with a human helper virus (eg, adenovirus): a plasmid containing a interested nucleic acid sequence flanked by two AAV inverted terminal repeats (ITR) region, and a plasmid carrying the AAV capsid genes (rep and cap genes). The resulting AAV recombinants are then purified by standard techniques.

In some embodiments, the recombinant vector is encapsidated into a virion (for example including but not limited to AAV1, AAV2, AAV3, AAV4, AAV5, AAV6, AAV7, AAV8, AAV9, AAV10, AAV11, AAV12, AAV13, AAV14, AAV15 and AAV16 in AAV virions). Accordingly, the present disclosure includes recombinant virions (recombinant in that they comprise a recombinant polynucleotide) comprising any of the vectors described herein. Methods of producing such particles are known in the art and described in U.S. Pat. No. 6,596,535.

### General Method

### Animal Ethics:

The breeding and use of animals in this study was completed under the guidance of the guidelines of the Biomedical Research Ethics Committee of the Center for Excellence in Brain Science and Intelligent Technology, Chinese Academy of Sciences.

### Plasmid Construction:

The plasmids in this study were all constructed by our laboratory. The AAV backbone vector was digested with restriction endonucleases, analyzed by agarose gel electrophoresis, and then recovered. The cell cDNA was used as a template to perform PCR, and the DNA fragment was recovered by agarose gel electrophoresis. According to standard operating procedures of ClonExpress MultiS One Step Cloning Kit (Vazyme, C113-02) from Nanjing vazyme Biotech Co., Ltd, the backbone vector was ligated with the fragment. After ligation, the plasmids were transformed into E. coli DH5α. Clones that are verified as correct through sequencing are then expanded for cultivation, and the plasmids are extracted. The constructions of the plasmids were as follows: AAV-GFAP-mCherry, AAV-GFAP-CasRx, AAV-GFAP-CasRx-gRNA(Pnky), CAG-Pnky-WPRE; CAG-CasRx, CMV-mCherry-U6-gRNA(Pnky), AAV-GFAP-tuna, AAV-GFAP-Let-7b, AAV-GFAP-miRNA-137, AAV-GFAP-miR-18b, AAV-GFAP-miR-24-3p, AAV-GFAP-miR-34a, AAV-GFAP-miR-92b, AAV-GFAP-miR-96, AAV-GFAP-miR-106, AAV-GFAP-miR-125a, AAV-GFAP-miR-128, AAV-GFAP-miR-134, AAV-GFAP-miR-135, AAV-GFAP-miR-141, AAV-GFAP-miR-143, AAV-GFAP-miR-184, AAV-GFAP-miR-200, AAV-GFAP-miR-218, AAV-GFAP-miR-219, AAV-GFAP-miR-429, AAV-GFAP-RMST, AAV-GFAP-Brn1b, AAV-GFAP-Dali, AAV-GFAP-Dlxas.

### Cell Culture and Transfection

293T cells were cultured in DMEM+10% fetal bovine serum+penicillin/streptomycin medium at 37° C. in an incubator with 5% CO₂ concentration. Transfection was performed when the cells grew to about 70% of the bottom of the culture dish. Cell transfection was carried out according to the standard operation procedure of EZ Trans cell transfection reagent (Shanghai Liji Biotech, AC04L092). The transfection plasmid in the experimental group was CAG-Pnky-WPRE+CAG-CasRx-EGFP+CMV-mCherry-U6-gRNA (Pnky), and the transfection plasmid in the control group was CAG-CasRx+CMV-mCherry-U6-gRNA (Pnky). Flow cytometric sorting was performed 48 hours after transfection, and 30,000 cells were collected from each sample. GFP and mCherry double-positive cells were collected from the experimental group, and GFP-positive cells were sorted from the control group. RNA was extracted from the collected cells using Trizol (Ambion), and its reverse transcription was performed using a reverse transcription kit (HiScript Q RT SuperMix for qPCR, Novozyme). AceQ qPCR SYBR Green Master Mix (Novazyme) was used for qPCR detection. The qPCR primers were: 5′-aggcagtgtgcggaggacat-3′ and 5′-gccattgtcctagcaagtgc-3′.

Injection of AAV into Mouse Brain:

The injection was performed using the RWD stereotaxic injection system. For the RNAs that needs to be down-regulated, the control group was injected with GFAP-mCherry+GFAP-CasRx, and the experimental group was injected with GFAP-mCherry+GFAP-CasRx-gRNA(Pnky). For the RNAs that needs to be upregulated, the control group was injected with GFAP-mCherry, and the experimental group was injected with GFAP-mCherry+GFAP-miRNA/LncRNA. In this experiment, the miRNA/LncRNA are GFAP-Tuna, GFAP-Let-7b and GFAP-miRNA-137. The titer of AAV mixture in each group was greater than 5×10¹² vg/ml (1-3 μl per injection). AAV was injected into striatum (AP+0.8 mm, ML±1.6 mm and DV-2.8 mm) or substantia nigra (AP-3.0 mm, ML±1.25 mm and DV-4.5 mm).

### Immunofluorescent Staining of Mouse Tissue:

1-2 months after AAV injection, the specimens were taken, sectioned and immunofluorescence stained. The approximate steps are: perfuse with normal saline first, then perfuse with 4% PFA, take out the target tissue and fix it with 4% paraformaldehyde (PFA) overnight, then dehydrate in 30% sucrose for at least 12 hours, and sliced after the tissue sinking into the bottom of the sucrose solution. Frozen sections were proceeded after OCT embedding with a slice thickness of 30 μm or 40 μm. Before immunofluorescent staining, the brain slices were washed three times with 0.1M phosphate buffered saline (PBS), each time for 5-10 minutes. After incubation with the primary antibody overnight at 4° C., the slices were washed 3-4 times with PBS, each time for 10-15 minutes. Subsequently, the secondary antibody diluted in antibody dilution buffer was added for incubation at room temperature for 2-3 hours. Then, the slices were washed again with PBS 3-4 times, each time for 10-15 minutes. Finally, the slides were mounted and preserved using anti-fade mounting medium (Life Technology).

Antibody Information Used in this Study is as Follows:

The primary antibodies used in this study included: guinea pig anti-NeuN (1:500, ABN90, Millipore), rabbit anti-TH (1:500, AB152, Millipore), rat anti-DAT (1:100, MAB369, Millipore) and mouse Anti-Flag (1:2000, F3165, Sigma). The secondary antibodies used in this study were: Cy5-AffiniPure Donkey Anti-Guinea Pig IgG (H+L) (1:500, 706-175-148, Jackson ImmunoResearch), Alexa Fluora-488 AffiniPure Donkey Anti-Rabbit IgG (H+L) (1:500, 711-545-152, Jackson ImmunoResearch), Alexa Fluora-488 AffiniPure Donkey Anti-Mouse IgG (H+L) (1:500, 715-545-150, Jackson ImmunoResearch) and Cy5 AffiniPure Donkey Anti-Rabbit IgG (H+L) (1:500, 711-175-152, Jackson ImmunoResearch).

### Sequence Information

Sequence Information of miRNAs

### Nucleotide Sequence of Let-7a

Human Let-7a coding sequence:
(SEQ ID NO: 1)
5′-tgaggtagtaggttgtatagtt-3′
Human Let-7a* coding sequence:
(SEQ ID NO: 2)
5′-ctatacaatctactgtctttc-3′
Human Let-7a-2* coding sequence:
(SEQ ID NO: 3)
5′-ctatacaatctactgtctttc-3′

The Full Length of Human Let-7a Pre-miRNA (there are Three Let-7a Pre-miRNAs in Humans)

Let-7a-1:
(SEQ ID NO: 4)
Tgggatgaggtagtaggttgtatagttttagggtcacacccaccactgg
gagataactatacaatctactgtctttccta
Let-7a-2:
(SEQ ID NO: 5)
Aggttgaggtagtaggttgtatagtttagaattacatcaagggagataa
ctgtacagcctcctagctttcct
Let-7a-3:
(SEQ ID NO: 6)
Gggtgaggtagtaggttgtatagtttggggctctgccctgctatgggat
aactatacaatctactgtctttcct

The core sequence of human Let-7a and mouse Let-7a (Let-7a-½) has 100% similarity at the DNA level.

### Nucleotide Sequence of Let-7b

Human Let-7b coding sequence:
(SEQ ID NO: 7)
5′-tgaggtagtaggttgtgtggtt-3′
Human Let-7b* coding sequence:
(SEQ ID NO: 8)
5′-ctatacaacctactgccttccc-3′

### The Full Length of Human Let-7b Per-miRNA

(SEQ ID NO: 9)
Cggggtgaggtagtaggttgtgtggtttcagggcagtgatgttgcccct
cggaagataactatacaacctactgccttccctg

The DNA level similarity between human and mouse Let-7b core sequences is 100%.

Nucleotide Sequence of miR-18

Human miR-18a coding sequence:
(SEQ ID NO: 10)
5′-taaggtgcatctagtgcagatag-3′
Human miR-18a* coding sequence:
(SEQ ID NO: 11)
5′-actgccctaagtgctccttctgg-3′
Human miR-18b* coding sequence:
(SEQ ID NO: 12)
5′-taaggtgcatctagtgcagttag-3′

The Full Length of Human miR-18 Per-miRNA

miR-18a
(SEQ ID NO: 13)
tgttctaaggtgcatctagtgcagatagtgaagtagattagcatctact
gccctaagtgctccttctggca
miR-18b
(SEQ ID NO: 14)
tgtgttaaggtgcatctagtgcagttagtgaagcagcttagaatctact
gccctaaatgccccttctggca

The core sequence of human miR-18a and mouse miR-18a is 100% similar at the DNA level.

Nucleotide Sequence of miR-24-3p

Human miR-24-3p coding sequence:
(SEQ ID NO: 15)
5′-tggctcagttcagcaggaacag-3′

The Full Length of Human miR-24-3p Per-miRNA (there are Two Coding Sequences in Humans)

miR-24-1
(SEQ ID NO: 16)
ctccggtgcctactgagctgatatcagttctcattttacacactggctc
agttcagcaggaacaggag
miR-24-2
(SEQ ID NO: 17)
ctctgcctcccgtgcctactgagctgaaacacagttggtttgtgtacac
tggctcagttcagcaggaacaggg

The core sequence of human miR-24-3p and mouse miR-24-3p is 100% similar at the DNA level.

Nucleotide Sequence of miR-34a

Human miR-34a coding sequence:
(SEQ ID NO: 18)
5′-tggcagtgtcttagctggttgt-3′
Human miR-34a* coding sequence:
(SEQ ID NO: 19)
5′-caatcagcaagtatactgccct-3′

The Full Length of Human miR-34a Per-miRNA

(SEQ ID NO: 20)
ggccagctgtgagtgtttctttggcagtgtcttagctggttgttgtgag
caatagtaaggaagcaatcagcaagtatactgccctagaagtgctgcac
gttgtggggccc

The core sequence of human miR-34a and mouse miR-34a is 100% similar at the DNA level.

Nucleotide Sequence of miR-92b

Human miR-92b coding sequence:
(SEQ ID NO: 21)
5′-agggacgggacgcggtgcagtg-3′
Human miR-92b* coding sequence:
(SEQ ID NO: 22)
5′-tattgcactcgtcccggcctcc-3′

The Full Length of Human miR-92b Per-miRNA

(SEQ ID NO: 23)
cgggccccggggggggggggacgggacgcggtgcagtgttgttttttccc
ccgccaatattgcactcgtcccggcctccggcccccccggccc

The core sequence of human miR-92b and mouse miR-92b has a similarity of 95.45% at the DNA level.

Nucleotide Sequence of miR-96

Human miR-96 coding sequence:
(SEQ ID NO: 24)
5′-tttggcactagcacatttttgct-3′
Human miR-96* coding sequence:
(SEQ ID NO: 25)
5′-aatcatgtgcagtgccaatatg-3′

The Full Length of Human miR-96 Per-miRNA

(SEQ ID NO: 26)
Tggccgattttggcactagcacatttttgcttgtgtctctccgctctgag
caatcatgtgcagtgccaatatgggaaa

The core sequence of human miR-96 and mouse miR-96 is 100% similar at the DNA level.

Nucleotide Sequence of miR-106

Human miR-106 coding sequence:
(SEQ ID NO: 27)
5′-aaaagtgcttacagtgcaggtag-3′
Human miR-106* coding sequence:
(SEQ ID NO: 28)
5′-ctgcaatgtaagcacttcttac-3′

The Full Length of Human miR-106 Per-miRNA

(SEQ ID NO: 29)
Ccttggccatgtaaaagtgcttacagtgcaggtagctttttgagatctac
tgcaatgtaagcacttcttacattaccatgg

The core sequence of human miR-106 and mouse miR-106 has a similarity of 91.3% at the DNA level.

Nucleotide Sequence of miR-128

Human miR-128 coding sequence:
(SEQ ID NO: 30)
5′-cggggccgtagcactgtctgaga-3′
Human miR-128* coding sequence:
(SEQ ID NO: 31)
5′-tcacagtgaaccggtctcttt-3′

The Full Length of Human miR-128 Per-miRNA

(SEQ ID NO: 32)
Tgagctgttggattcggggccgtagcactgtctgagaggtttacatttct
cacagtgaaccggtctctttttcagctgcttc

The core sequence of human miR-128 and mouse miR-128 is 100% similar at the DNA level.

Nucleotide Sequence of miR-134

Human miR-134 coding sequence:
(SEQ ID NO: 33)
5′-tgtgactggttgaccagagggg-3′
Human miR-134* coding sequence:
(SEQ ID NO: 34)
5′-cctgtgggccacctagtcaccaa-3′

The Full Length of Human miR-134 Per-miRNA

(SEQ ID NO: 35)
Cagggtgtgtgactggttgaccagaggggcatgcactgtgttcaccctgt
gggccacctagtcaccaaccctc

The core sequence of human miR-134 and mouse miR-134 is 100% similar at the DNA level.

Nucleotide Sequence of miR-135

Human miR-135 coding sequence:
(SEQ ID NO: 36)
5′-tatggctttttattcctatgtga-3′
Human miR-135* coding sequence:
(SEQ ID NO: 37)
5′-tatagggattggagccgtggcg-3′

The Full Length of Human miR-135 Per-miRNA

(SEQ ID NO: 38)
Aggcctcgctgttctctatggctttttattcctatgtgattctactgctc
actcatatagggattggagccgtggcgcacggggggaca

The core sequence of human miR-135 and mouse miR-135 is 100% similar at the DNA level;

Nucleotide Sequence of miR-137

Human miR-137 coding sequence:
(SEQ ID NO: 39)
5′-acgggtattcttgggtggataat-3′
Human miR-137* coding sequence:
(SEQ ID NO: 40)
5′-ttattgcttaagaatacgcgtag-3′

The Full Length of Human miR-137 Per-miRNA

(SEQ ID NO: 41)
Ggtcctctgactctcttcggtgacgggtattcttgggtggataatacgga
ttacgttgttattgcttaagaatacgcgtagtcgaggagagtaccagcgg
ca

The core sequence of human miR-137 and mouse miR-137 is 100% similar at the DNA level;

Nucleotide Sequence of miR-141

Human miR-141 coding sequence:
(SEQ ID NO: 42)
5′-catcttccagtacagtgttgga-3′
Human miR-141* coding sequence:
(SEQ ID NO: 43)
5′-taacactgtctggtaaagatgg-3′

The Full Length of Human miR-141 Per-miRNA

(SEQ ID NO: 44)
Cggccggccctgggtccatcttccagtacagtgttggatggtctaattgt
gaagctcctaacactgtctggtaaagatggctcccggggggttc

The core sequence of human miR-141 and mouse miR-141 has a similarity of 95.45% at the DNA level.

Nucleotide Sequence of miR-143

| | Human miR-143 coding sequence: |
|---|---|
| (SEQ ID NO: 45) | |
| | 5′-ggtgcagtgctgcatctctggt-3′ |
| | Human miR-143* coding sequence: |
| (SEQ ID NO: 46) | |
| | 5′-tgagatgaagcactgtagctc-3′ |

The Full Length of Human miR-143 Per-miRNA

(SEQ ID NO: 47)
Gcgcagcgccctgtctcccagcctgaggtgcagtgctgcatctctggtca
gttgggagtctgagatgaagcactgtagctcaggaagagagaagttgttc
tgcagc

The core sequence of human miR-143 and mouse miR-143 is 100% similar at the DNA level.

Nucleotide Sequence of miR-184

| | Human miR-184 coding sequence: |
|---|---|
| (SEQ ID NO: 48) | |
| | 5′-ccttatcacttttccagcccagc-3′ |
| | Human miR-184* coding sequence: |
| (SEQ ID NO: 49) | |
| | 5′-tggacggagaactgataagggt-3′ |

The Full Length of Human miR-184 Per-miRNA

(SEQ ID NO: 50)
ccagtcacgtccccttatcacttttccagcccagctttgtgactgtaagt
gttggacggagaactgataagggtaggtgattga

The core sequence of human miR-184 and mouse miR-184 has a similarity of 95.45% at the DNA level.

Nucleotide Sequence of miR-200

| | Human miR-200 coding sequence |
|---|---|
| | miR-200a: |
| (SEQ ID NO: 51) | |
| | 5′-catcttaccggacagtgctgga-3′ |
| | miR-200b: |
| (SEQ ID NO: 52) | |
| | 5′-catcttactgggcagcattgga-3′ |
| | miR-200c: |
| (SEQ ID NO: 53) | |
| | 5′-cgtcttacccagcagtgtttgg-3′ |
| | Human miR-200 coding sequence |
| | miR-200a: |
| (SEQ ID NO: 54) | |
| | 5′-taacactgtctggtaacgatgt-3′ |
| | miR-200b: |
| (SEQ ID NO: 55) | |
| | 5′-taatactgcctggtaatgatga-3′ |
| | miR-200c: |
| (SEQ ID NO: 56) | |
| | 5′-taatactgccgggtaatgatgga-3′ |

The Full Length of Human miR-200 Per-miRNA

miR-200a:
(SEQ ID NO: 57)
ccgggcccctgtgagcatcttaccggacagtgctggatttcccagcttg
actctaacactgtctggtaacgatgttcaaaggtgacccgc
miR-200b:
(SEQ ID NO: 58)
ccagctcgggcagccgtggccatcttactgggcagcattggatggagtc
aggtctctaatactgcctggtaatgatgacggcggagccctgcacg
miR-200c:
(SEQ ID NO: 59)
ccctcgtcttacccagcagtgtttgggtgcggttgggagtctctaatac
tgccgggtaatgatggagg

The core sequences of human miR-200a/b/c and mouse miR-200a/b/c are 100% similar at the DNA level.

Nucleotide Sequence of miR-218

| | Human miR-218-1 coding sequence: |
|---|---|
| (SEQ ID NO: 60) | |
| | 5′-ttgtgcttgatctaaccatgt-3′ |
| | Human miR-218-1* coding sequence: |
| (SEQ ID NO: 61) | |
| | 5′-atggttccgtcaagcaccatgg-3′ |
| | Human miR-218-2* coding sequence: |
| (SEQ ID NO: 62) | |
| | 5′-catggttctgtcaagcaccgcg-3′ |

The Full Length of Human miR-218 Per-miRNA

miR-218-1:
(SEQ ID NO: 63)
gtgataatgtagcgagattttctgttgtgcttgatctaaccatgtggttg
cgaggtatgagtaaaacatggttccgtcaagcaccatggaacgtcacgca
gctttctaca
miR-218-2:
(SEQ ID NO: 64)
gaccagtcgctgcggggctttcctttgtgcttgatctaaccatgtggtgg
aacgatggaaacggaacatggttctgtcaagcaccgcggaaagcaccgtg
ctctcctgca

The core sequences of human miR-218-1/2 and mouse miR-218-1/2 are 100% similar at the DNA level.

Nucleotide Sequence of miR-219

| | Human miR-219-1 coding sequence: |
|---|---|
| (SEQ ID NO: 65) | |
| | 5′-tgattgtccaaacgcaattct-3′ |
| | Human miR-219-1* coding sequence: |
| (SEQ ID NO: 66) | |
| | 5′-agagttgagtctggacgtcccg-3′ |
| | Human miR-219-2 coding sequence: |
| (SEQ ID NO: 67) | |
| | 5′-tgattgtccaaacgcaattct-3′ |
| | Human miR-219-2* coding sequence: |
| (SEQ ID NO: 68) | |
| | 5′-agaattgtggctggacatctgt-3′ |

The Full Length of Human miR-219 Per-miRNA

miR-219-1:
(SEQ ID NO: 69)
ccgccccgggccgcggctcctgattgtccaaacgcaattctcgagtctat
ggctccggccgagagttgagtctggacgtcccgagccgccgcccccaaac
ctcgagcggg
miR-219-2:
(SEQ ID NO: 70)
actcaggggcttcgccactgattgtccaaacgcaattcttgtacgagtct
gcggccaaccgagaattgtggctggacatctgtggctgagctccggg

The core sequences of human miR-219-1/2 and mouse miR-219-1/2 is 100% similar at the DNA level.

Nucleotide Sequence of miR-429

| | Human miR-429 coding sequence: |
|---|---|
| (SEQ ID NO: 71) | |
| | 5′-gtcttaccagacatggttaga-3′ |
| | Human miR-429* coding sequence: |
| (SEQ ID NO: 72) | |
| | 5′-taatactgtctggtaaaaccgt-3′ |

The Full Length of Human miR-429 Per-miRNA

(SEQ ID NO: 73)
cgccggccgatgggcgtcttaccagacatggttagacctggccctctgtc
taatactgtctggtaaaaccgtccatccgctgc

The core sequence of human and mouse miR-429 is 100% similar at the DNA level.

Nucleotide Sequence of miR-430

| | Human miR-430 coding sequence: |
|---|---|
| (SEQ ID NO: 74) | |
| | 5′-acttaaacgtggatgtacttgct-3′ |
| | Human miR-430* coding sequence: |
| (SEQ ID NO: 75) | |
| | 5′-taagtgcttccatgttttggtga-3′ |

The Full Length of Human miR-430 Per-miRNA

(SEQ ID NO: 76)
ccaccacttaaacgtggatgtacttgctttgaaactaaagaagtaagtgc
ttccatgttttggtgatgg

Nucleotide Sequence of miR-7a

| | Human miR-7a coding sequence: |
|---|---|
| (SEQ ID NO: 77) | |
| | 5′-tggaagactagtgattttgttgtt-3′ |
| | Human miR-7a* coding sequence: |
| (SEQ ID NO: 78) | |
| | 5′-caacaaatcacagtctgccata-3′ |

The Full Length of Human miR-7a Per-miRNA

(SEQ ID NO: 79)
ttggatgttggcctagttctgtgtggaagactagtgattttgttgttttt
agataactaaatcgacaacaaatcacagtctgccatatggcacaggccat
gcctctacag

The core sequence of human miR-7a and mouse miR-7a is 100% similar at the DNA level.

Nucleotide Sequence of miR-15

Human miR-15-1 coding sequence:
(SEQ ID NO: 80)
5′-tagcagcacataatggtttgtg-3′
Human miR-15-1* coding sequence:
(SEQ ID NO: 81)
5′-caggccatattgtgctgcctca-3′
Human miR-15-2 coding sequence:
(SEQ ID NO: 82)
5′-tagcagcacatcatggtttaca-3′
Human miR-15-2* coding sequence:
(SEQ ID NO: 83)
5′-cgaatcattatttgctgctcta-3′

The Full Length of Human miR-15 Per-miRNA

miR-15-1:
(SEQ ID NO: 84)
ccttggagtaaagtagcagcacataatggtttgtggattttgaaaaggt
gcaggccatattgtgctgcctcaaaaatacaagg
miR-15-2:
(SEQ ID NO: 85)
ttgaggccttaaagtactgtagcagcacatcatggtttacatgctacag
tcaagatgcgaatcattatttgctgctctagaaatttaaggaaattcat

The core sequence of human miR-15 and mouse miR-15 is 100% similar at the DNA level.

Nucleotide Sequence of miR-23

Human miR-23 coding sequence:
(SEQ ID NO: 86)
5′-ggggttcctggggatgggattt-3′
Human miR-23* coding sequence:
(SEQ ID NO: 87)
5′-atcacattgccagggatttcc-3′

The Full Length of Human miR-23 Per-miRNA

(SEQ ID NO: 88)
ggccggctggggttcctggggatgggatttgcttcctgtcacaaatcac
attgccagggatttccaaccgacc

The core sequence of human miR-23 and mouse miR-23 is 100% similar at the DNA level.

Nucleotide Sequence of miR-25

Human miR-25 coding sequence:
(SEQ ID NO: 89)
5′-aggcggagacttgggcaattg-3′
Human miR-25* coding sequence:
(SEQ ID NO: 90)
5′-cattgcacttgtctcggtctga-3′

The Full Length of Human miR-25 Per-miRNA

(SEQ ID NO: 91)
ggccagtgttgagaggcggagacttgggcaattgctggacgctgccctg
ggcattgcacttgtctcggtctgacagtgccggcc

The core sequence of human miR-25 and mouse miR-25 is 100% similar at the DNA level.

Nucleotide Sequence of miR-29a

Human miR-29a coding sequence:
(SEQ ID NO: 92)
5′-actgatttcttttggtgttcag-3′
Human miR-29a* coding sequence:
(SEQ ID NO: 93)
5′-tagcaccatctgaaatcggtta-3′

The Full Length of Human miR-29a Per-miRNA

(SEQ ID NO: 94)
atgactgatttcttttggtgttcagagtcaatataattttctagcacca
tctgaaatcggttat

The core sequence of human miR-29a and mouse miR-29a is 100% similar at the DNA level.

Nucleotide Sequence of miR-129

Human miR-129-1 coding sequence:
(SEQ ID NO: 95)
5′-ctttttgcggtctgggcttgc-3′
Human miR-129-1* coding sequence:
(SEQ ID NO: 96)
5′-aagcccttaccccaaaaagtat-3′
Human miR-129-2 coding sequence:
(SEQ ID NO: 97)
5′-ctttttgcggtctgggcttgc-3′
Human miR-129-2* coding sequence:
(SEQ ID NO: 98)
5′-aagcccttaccccaaaaagcat-3′

The Full Length of Human miR-129 Per-miRNA

miR-129-1:
(SEQ ID NO: 99)
ggatctttttgcggtctgggcttgctgttcctctcaacagtagtcagga
agcccttaccccaaaaagtatct
miR-129-2:
(SEQ ID NO: 100)
tgcccttcgcgaatctttttgcggtctgggcttgctgtacataactcaa
tagccggaagcccttaccccaaaaagcatttgcggagggcg

The core sequence of human miR-129-1/2 and mouse miR-129-1/2 is 100% similar at the DNA level.

Nucleotide Sequence of miR-137

Human miR-137 coding sequence:
(SEQ ID NO: 101)
5′-acgggtattcttgggtggataat-3′
Human miR-137* coding sequence:
(SEQ ID NO: 102)
5′-ttattgcttaagaatacgcgtag-3′

The Full Length of Human miR-137 Per-miRNA

(SEQ ID NO: 103)
ggtcctctgactctcttcggtgacgggtattcttgggtggataatacgg
attacgttgttattgcttaagaatacgcgtagtcgaggagagtaccagc
ggca

The core sequence of human miR-137 and mouse miR-137 is 100% similar at the DNA level.

Nucleotide Sequence of miR-138

Human miR-138-1 coding sequence:
(SEQ ID NO: 104)
5′-agctggtgttgtgaatcaggccg-3′
Human miR-138-1* coding sequence:
(SEQ ID NO: 105)
5′-gctacttcacaacaccagggcc-3′
Human miR-138-2 coding sequence:
(SEQ ID NO: 106)
5′-agctggtgttgtgaatcaggccg-3′
Human miR-138-2* coding sequence:
(SEQ ID NO: 107)
5′-gctatttcacgacaccagggtt-3′

The Full Length of Human miR-138 Per-miRNA

miR-138-1:
(SEQ ID NO: 108)
ccctggcatggtgtggtggggcagctggtgttgtgaatcaggccgttgc
caatcagagaacggctacttcacaacaccagggccacaccacactacag
g
miR-138-2:
(SEQ ID NO: 109)
cgttgctgcagctggtgttgtgaatcaggccgacgagcagcgcatcctc
ttacccggctatttcacgacaccagggttgcatca

The core sequence of human miR-138-1/2 and mouse miR-138-1/2 is 100% similar at the DNA level.

Nucleotide Sequence of miR-155

Human miR-155 coding sequence:
(SEQ ID NO: 110)
5′-ttaatgctaatcgtgataggggtt-3′
Human miR-155* coding sequence:
(SEQ ID NO: 111)
5′-ctcctacatattagcattaaca-3′

The Full Length of Human miR-155 Per-miRNA

(SEQ ID NO: 112)
ctgttaatgctaatcgtgataggggtttttgcctccaactgactcctac
atattagcattaacag

The core sequence of human miR-155 and mouse miR-155 has a similarity of 95.8% at the DNA level.

Nucleotide Sequence of miR-195

Human miR-195 coding sequence:
(SEQ ID NO: 113)
5′-tagcagcacagaaatattggc-3′
Human miR-195* coding sequence:
(SEQ ID NO: 114)
5′-ccaatattggctgtgctgctcc-3′

The Full Length of Human miR-195Per-miRNA

(SEQ ID NO: 115)
agcttccctggctctagcagcacagaaatattggcacagggaagcgagt
ctgccaatattggctgtgctgctccaggcagggtggtg

The core sequence of human miR-195 and mouse miR-195 is 100% similar at the DNA level.

Nucleotide Sequence of miR-214

Human miR-214 coding sequence:
(SEQ ID NO: 116)
5′-tgcctgtctacacttgctgtgc-3′
Human miR-214* coding sequence:
(SEQ ID NO: 117)
5′-acagcaggcacagacaggcagt-3′

The Full Length of Human miR-214Per-miRNA

(SEQ ID NO: 118)
ggcctggctggacagagttgtcatgtgtctgcctgtctacacttgctgt
gcagaacatccgctcacctgtacagcaggcacagacaggcagtcacatg
acaacccagcct

The core sequence of human miR-214 and mouse miR-214 is 100% similar at the DNA level.

Nucleotide Sequence of miR-222

Human miR-222 coding sequence:
(SEQ ID NO: 119)
5′-ctcagtagccagtgtagatcct-3′
Human miR-222* coding sequence:
(SEQ ID NO: 120)
5′-agctacatctggctactgggt-3′

The Full Length of Human miR-222 Per-miRNA

(SEQ ID NO: 121)
gctgctggaaggtgtaggtaccctcaatggctcagtagccagtgtagat
cctgtctttcgtaatcagcagctacatctggctactgggtctctgatgg
catcttctagct

The core sequence of human miR-222 and mouse miR-222 is 100% similar at the DNA level.

Nucleotide Sequence of miR-223

Human miR-223 coding sequence:
(SEQ ID NO: 122)
5′-cgtgtatttgacaagctgagtt-3′
Human miR-223* coding sequence:
(SEQ ID NO: 123)
5′-tgtcagtttgtcaaatacccca-3′

The Full Length of Human miR-223 Per-miRNA

(SEQ ID NO: 124)
cctggcctcctgcagtgccacgctccgtgtatttgacaagctgagttgg
acactccatgtggtagagtgtcagtttgtcaaataccccaagtgcggca
catgcttaccag

The core sequence of human miR-223 and mouse miR-223 is 100% similar at the DNA level.

Nucleotide Sequence of miR-132

Human miR-132 coding sequence:
(SEQ ID NO: 125)
5′-accgtggctttcgattgttact-3′
Human miR-132* coding sequence:
(SEQ ID NO: 126)
5′-taacagtctacagccatggtcg-3′

The Full Length of Human miR-132 Per-miRNA

(SEQ ID NO: 127)
ccgcccccgcgtctccagggcaaccgtggctttcgattgttactgtggg
aactggaggtaacagtctacagccatggtcgccccgcagcacgcccacg
cgc

The core sequence of human miR-132 and mouse miR-132 is 100% similar at the DNA level.

Mir-133

[symbol]

[symbol]

miR-133 [symbol] :
(SEQ ID NO: 128)
5′-agctggtaaaatggaaccaaat-3′ [symbol]
miR-133* [symbol] :
(SEQ ID NO: 129)
5′-tttggtccccttcaaccagctg-3′

The Full Length of Human miR-133 Per-miRNA

(SEQ ID NO: 130)
acaatgctttgctagagctggtaaaatggaaccaaatcgcctcttcaat
ggatttggtccccttcaaccagctgtagctatgcattga

The core sequence of human miR-133 and mouse miR-133 has 100% similarity at the DNA level.

Sequence Information of lncRNA

Human-Pnky
(SEQ ID NO: 131)
aggcgccaggggcccggttggcgcgaacgccgggttccgagcaccctgggcttccttgtctgcctcccagcgcggcacctcttcggggctcccgaaacctgag
ctctcgctggttttaggtccagactggggcctctccaccggttcctcccccgccccgggctctggggcccattctttgggctgaccctgtcagggcagagtccgcgcgtctgc
ctgccattctccgcccgcataaaagcacgttgaaggtgtctcgggcagacacctccaggttttgaatcagtttattccctttcactgttcaaagcagctgttcaaatacacaggct
gcttacgttgacgtggagaggatttcaaacaacgctaaaatgctttgaactgacaaggtgtcttgatatctccctcactccatccagcacagctcctcgagatcactcgctagga
caatggctgagcaggcgattcgtgcgggcctcgccacctcggggcgcggactgcggggtgtcctaagccccttccgcaaggacaggatggaggcacctgtaaggagat
gctggcgccaccccagcttctcccaggtccggaggaacctctactcagtcaataccctgagctggacttgtctgaagaaacggagccgactccctcttgccggggtgcgct
gagtggaggggaaacatcctcgaataacagaactacaccaaaaagacacccatgttatctctcacactttcacactcctcgagatagtgagccggacctgggtcttagtagca
cccagtaccttgacacaaacctcccaaatttccacctgagtaacagttatggggtcagtccatgcactgtaacttgaactctaatttattaactatttcatctagtaaacacactcac
accatatataaaatagcatttatttatttctatataccaggagttggcagaaaacccaccgtgaccactcccatacattgagctggaggcacacaattactaaaacagaggtgaa
atggtattcatttgatcttaattttttcttatttatgtagtcccaggataatagaaatcaggaaacaaaagaaaacaaagaattttctgaggagatggccattgggggagtggaggt
agcagctggtttaaacctaagtaaaactagaaaaagaaactgctgtttcctttttcttatatccaccttagaggatcatgtttgaacgtccctactcctcctcctctttttaaaaagcct
tgtctcagtcattcattcctgtgcttcctgctcttctgctagaccccagcagctgtttgatttggtgaggcccccctccaacctctgagtggaacttcttttctaagggcctgcagaa
tgtcaaaactgaggctctggcttcggagctagagctttgaacagccaatccacacaaaaaggcagctggctgctttaatgaaaactgctataaagcttcaagaactttagcctt
gggggatgcatttataaggaacatggaaaatgcatttccaagttgctggttcttgggagagacataataaacatttacc
Human-Paupar
(SEQ ID NO: 132)
aaaggtaccggaataaaaatttgactaaagtttgggataaattaatcggtggcttgaagaacagtagcacatttctgattatgtaggctccagccaggatttaagttttg
tgtcatgattgttttcatatttctttgacatctattgatttggaggcagctgcaagcctttgtcacaggagagaagagcagtcacaatatattagcttgatggcataactcgaaccgg
agaagcgaggggagcctgagtgactggggcgcacatgacccccctcctggggcctttgggctcaacccttacttttaacttgttctaacaggaataatggagtagtactacta
atgcatggtcagaaggggcttttctgtctagaacgcaggcggcgggagttgcaccgccacagcccgggagaccaaagagtctccactccccgctcccggcgcctcctcgc
cgaccggagccccgagcccagtgagcccagccggtcccacgtcgagaggcgctgggttgggagaagttctgctggtcgtgggctcggcccccaggcgccaggccgag
tgcccacctcggcttctttagggggcctacagcgggcagccgagcggctggcggacccggagcttgggaggcgaccgccaggctggtgcccggcctgacccggcgttc
gcggccgcccgctcgcccgccgcgggccgggagcgtacaggagtgtgacgcagattgtgaaaacagaagggagggagttgggtcatttccttcgctaatcatcgccccc
tcctgctcctcctccccacccccatcccatcctcgccgccacccagcctcgaactctcttccttgccagagtctgcctcactttgagccagtggctgcactttatggagaaaatat
atgtgcccatcactcacccatctatttgcagcgtccatggagcgcctcggggaacacgcatggacaattaaacacacccacttacgggggatgatgaggctttctgttaacc
acatgtgttcagaaatggatacagccctaattttgacacggaatgcagctaatgcaccttgctatagatgtacgtccgttggaaaagtgtgtgatgggcacctgatctacaccaa
gagaacagggctgtctgcagaagacccagcggcctgacaccttttcttgttgccggggaaggaaagtttttttttgatttgtcctctcacgatttttcccttttcctttgcattacag
tttcagagaggaatgtaaatattttgactagacctctgcaggggccttttaaacaccaggcacaatcatctgtggaatactgtagttcatactttcaaaacttgggtgttaaagagt
ctgtcttgctgaggaaacccatagaactagtggcgacctttcaactccctatttgtgtgatgaaatgaagtatgccctagtgattgtcgaattttaaggacttgttctgtcactgcta
ctttccgatcgtttgcagggaagaaaaatcaatcaaacagacctttgatggggttgtggcaaggtcacatttcactgtgctccaggcttacctcttcttaacgcccctccctgctgt
ggagcgtgtgcaggtagtatgcaaaactctagaaataatatttgaaaaaattaacaccttgtttatctcttctgtgtatttgatataggggtttctttcttttcctgttttggtgtttaatac
aaggctgatatattggaacagaatgcagacttcaaagaaactaaaattaggaggcaaatagcgtcaaggtttcctcggtttctgtatgtagtacaaatagtatagcgtcttttcatc
caaaaagaagcaaaaattgccaggaggcataaagcatctctgaatatccgttttttttaaagaaacaaacatttcccaaatattgatgtacgcatggttttatagtctgaaaatattt
ctaggcacaaatatggcatacaaacattcgtttcaactctgtcgttgttgttaaaatgacagtaaattatagtttattttaagatacctccctgttaatgtcagttgaggttattttaggg
cctggatgctctaggaagaccacctaaaatgtgtttatttctctgcccaattcacctatagtgtaaggagcattttattttattttattttattttattttattttattatactttaagttttaggg
tacatgtgcacaatgtgcaggtttgttacatgtgtatacttgtgccatgttggtgtgatgcacccattaactcgtcatttagcattaggtatatctcctaatgctatccctcccctcccc
ccatcccacaacagtccccagagtgtcatgtaaggagcattttaaataaaatattttgggcatccttaagtccttttttaaaaatccaacattctacaattgtgacaaataggctgaa
aaattttgttttgtttataccaaattcattttaataaagcatttgctgagattttaatacaacaaatgccaacaaaatgcatcagagtagaaaaaagaaaatattttcagaattgcctata
atattgcttgtgatcctaaattttttgatgcaaggaacttgaatttttcagctaatattattctgttttcttattctgtgtttctattagaacactcacctatgctctgcttcccttgctgtcttcc
catagcttatgtgtgcactatgaatttcaataaatataaattactgcttaaaagaaaaaaaaaaaaaaaaaa
human-HOTAIRM1
(SEQ ID NO: 133)
aaaagtttgccggcttccgcagtgatggatcaccgttttagtggcatttaaatccccggcgctccgccgtctaggtgacgcgcagtcgcccccccaggcagcctag
gcggcggcagctgctgcggcgactgcaaaggccgatttggagtgctggagcgaagaagagcaaaagctgcgttctgcgcgcgcccgactccgctgcccgccccgcca
ggcctccgggaggtgggggctgggaggcgtcccccgctcccgccccctccccaccgttcaatgaaagatgaactggcgagaggacgaatcgcatccaggagctgcgca
gccctggccgctgccgggacgccctgctccgcgctgagcttggggccagaaaccagccatagtccccacactccgccgccgcagctgagatttagcggaggaaggggc
gagggaaggtagggagcaaacctatgaagaaacatcgcgttgtcattggaacttccaagcctttgctgttaagagccaggttcttaaatcaacccgccccacacacatgttgc
ttacatgctgcgttttctcacggtctgttttgcctgaacccatcaacagctgggagattaatcaaccacactgaaaatgtggagggatttatgggggagggggttgaaatgtggg
tgtttgaaacaaaagtgtataaacaaatgaattgttgataacttagttattgacctggagactggtagcttattaaagaaactccgtgttactcattcctggagttgggggtttctgta
ggcactttatttctccactttcaagagcttgggcttggcccaaatcttagactgtccaattctgcctctattaccaatttaaatctatggcttgaacctgtgcactgaaaatcaaatcct
ttaaaaagaaagaggagaagaagaagcaaaaaagaaagaaaaaacacttattagaagccctagtcattttttggctttctgttttgttgctgtccattgaagactttgaacatgcc
gccttaataaatgtattaaaattgaaaaaagaaaaaaaaa
human-TUG1
(SEQ ID NO: 134)
gagcgacgcagccgggacggtagctgcggtgcggaccggaggagccatcttgtctcgtcgccggggagtcaggcccctaaatcgaagaagccctggcgcgc
cctccccccctcccgggtctgatagcagactccttgaaagcagggtccttgtttagtgcatctttgcccacatacaccacaacatatcaagatgcatttattaggaaggaggagt
ttagagagcaggctatcagaataaccactcatcctgtgcctcctgattgctgagtgttcacctggaccttctgactaccttccctgtgctattccatcagcctacagacctggtacc
tggatttttgcccgagatgattcctaccaccttactactgacgaagacacccattccagtggaccactgtgacccaggaggcattcagccatcatgatgtggcctttacctccac
tcctgtcttgttctacccagattcagcacagccctttatagtgaagtcagagtcctcaagccaaatagctaaagctgttttatcacaacaaaggcctagtttgttccatgagtgtgc
atttcatttcttcagttaaagccttcagagacacacaataaatttggaccaggggattttttagttattaatgctctctgaagaaaggcaacatctttttgagagcagcattggacca
caccccacaatctcaaatgattgaaattcatgaacatctaggatcccgtgaaggtcactggaccctgttttttctacttcaaatcctgtagtagcctactgaatgagaaaacatatt
ctgacccattgggatcaaatcaaaggcacagtgaactcctcatagcatcttctttggaattactcaggaaccagaactttttacacaaatgtaagaaattctaccaaggagtcccc
ttacctaacagcatctcacaaggctgcaccagattccagaaaaggcttctcttgatacatcaagcattttgtgaccgacttattcttagatcattggttttccaaaggctttgtggcc
atgaagccctttgagtgaaaactgtgcagaagcccagagtaaaagtgaagctgctctggatgaagtagtgaagcaagagtaggggcctgaatcctgctacaactatcttcctt
taccaccgtggtgacacctaaggggacttccttacaacaccttgaactcttccgaacacagtttgaaaaccactgccccagacagcaatatgtttgacctgaatggcattccaat
cttttctgtacctccactcagcacagttcatgttcagtagatgctgaacattcttagaaatactgtgtgtgaacttagaaaagtgcaagaagacaggcatgtctttgaccccagga
atgatcatttgctgaagatggtgtcaagtgaacctagattaacagccctccactccagatggatatccagtgattcctagaatgggatatagccagagaacaattctatgcaccc
tacactgacagactcccttaagcaacaccagatgctctactggtacttgaagtacatgactttgaagtcttgaccctccatgaatacctgaattatcagcaagcgggttttgaagc
tggtgcctcattgaggccatattagagcaacttgtacatttgacctcttgttatcagccatggtactctacttcgtgtgcaagagataactatgaaagccaaattcaaatactggca
acatttcctaaaggggctcaatatctatcattcgtcttcttttccaaactacacatcactgtatgactcaaccagtagcagttatattgccccttggtttttattcagtttaactactgtttc
caagataaatgagctaataagctttaaaaaaaaaaaaaaaaaaggctgaattcttttttcttcatcactggcatatctgcctattctccagaattattatgactattcagctcactttaa
cagttgaacttcaagcgacaatctttgaacaccccttctcatgtgatttaaaatgaaaccatttggaaaagtttcttctagccagtaatagattttttttttaattgctctgccttgtgcc
gagagatgttcttttaagatgaatcttttgatgtctgataccaccaaatataggtggtagggagagttggaggctggccctttgagcaggccattagcttacttgctgggcatttcc
gatagcttattgcctacctttttgctggaaacaaactgatttgaaaaaaaaatctatgaagactgcagctaaggattttatcggtagacttaagagcttttgtccttgtggatatttta
gtggaaccacatcagtctcaatactgtcattttacactgactcagagcagctgacttcattccttgccatgatatatatttaaggcaggcattgtaacagacataaagacaacttat
ctgtttcagcaggaaggattcagtttatgaactctcagaccagatcatgttgaacaaggagactttgatgtgtgtcatgagaaaactcattctttacttcccagtcaatttaaaggc
cagctatcctgagctactcgaatgaatgcactggttaaacattggaaatagtttgtttatatccttgtctctctctaggccaattgtgattacatgactcgactctacatctcgtcaaa
caaggcctaggtctggttgctgtagactgctcgccctcaacaaataaaatctggttgactagcctccttgtatatacaactattatttgttaagaagaaattatcgtcaattttctact
accttccaattgtcagctctttttttcctctctggtttttcctatactttacagaaaaagacattgatctatactgccattccctctaatcctgccatactcagtcaaaaggaatgacttaa
gatgaagatgatcatctgctcgagtctaaaatatacattgtatataagaattggtgattagaaaagcaaaaaacctaaaacttaaatctaggagtctgtatactgtctccatgtctcc
atgcctcagatctcatctaaatctttgaacagcaccattcaaccaatctgaggccttgacttgcttgtaagatgattctcagagatcggctgagttaaaaaagatgacgacttgatt
accaaagaaagtagggccaactttgacaaatctggctctgctgaccctgtcactcccagatgtagcatagactcctaaacagaacctcaagtctgattgaggataaggccttct
cctgagctgaaagttctttggcagatgagcaagaaactgaaagctgatgtacctgactggctctgtaagatcagaaaactgtatccagaataagccctatggattaacccctga
gtacccagagtaaaaactaatttacagaacttccttattgatctgctggttcttccagatcatattctggctattggtatggctggcctttctgaaggtaccctgcttgtctattttcctg
actcagctcttgcctgcctttttcacatgttgctgcaattagactcaccgtgaggactacagtcaatttcagtctatcttgtgcccaatacaacaaggatttttaatagtaacaaccca
cacctcacccactaggactcaatgttcacaacaggaaggaccattgctgcatactccttgaccagcaacttttttgaagatatttttaagtgcagagtaggcctctattcctgtatg
taattgttcattttcagcacctggaacctcatctatcgggtctggaaggaatacagcagttcgaaagccgcgtccatttctctccttcagtagtgcagaaatgagtccgattcacc
agtacacacagaactgtaccagttcaacctagcaaaagaagaaaagtttccactgtacttaaaatttacagctgactcaaattgcctcacagaattatttgatgtagaaggctagt
tgtcttacttcagatcagcaggacagttgggctctcagactcatgaccactgagtttgcttgtgttgaaactgtggtttcatccaacatatgctattggacatgattattattccattca
aatggattacagacttcttgaggacaggacaaacttatctctcatggtgtttttttagaatacttttataaccaaggaagaaaccatgccagctgttaccattcaacttcttaagcag
agattaagctttttcatatctgttcttatcctggacatcagtagtttttaattgcccagcatccgttccatcttgtaacaactccctgatgtttcttaaaaccacctcttcctattttcagtct
gtggtttggacagtctgacccaaccttgagctttgtgggtgaacatgtaattcagacctcatcaatcagcaaatccatctgaactgtggaggagaagctctctttactgagggtg
ctttagctttgtaggatgaaaacctcaaactaacagggcctaccatgtagagaatgaagccagtgcaggggaaagcagagccaaaatatggagagacttgaatcctgatgac
agcgtttgtgcccctggatccaaccgtgcctgaagctagaatatcccctggacttttcagttatgtgaaccaataaatacccttttttgcttaagttactttgagttgggtttctgttac
ttgaaattgaatccacactaatatatctaccaacattgagacttgacagatccaagtatttattaagctagaggtcatggtcactgaaattactttccaaagtggaagacaaaatga
aacaggaactgagggaatatttaagatcccacagaagcgtaaaaatgacatggtagaaagtaatagaaaacctaaatgtctgtcattaaaggataggttaaggtgtggttcag
ccatataggaatatctcgtatctgttaaaatgaataaagtacattcattgtgtatggaaaaatggccatgatacattaggtgaaacaagttattaatagaaaagtgtacagtgtgaa
ctcattttaaaatgtgtgtgcttatgtttataaatgcatagaaaggtctattcacagctttctttgaacagtgtagatcacatgaaactttcaactttatacatttctgtattaatattttaca
ctacccacattatttttaaactttattttaaataaagaatttttaaaattaaa
Human-RMST
(SEQ ID NO: 135)
atttcatttgtgattcggatgatgcagctctaggtggattgactatgaaaggcgctgaatatcttcaggaaaatgggttccatgagaagtgacatcctcctcgaatcaa
aagattctcaggatgatggagtgagtgatggaataggttgccaactgtagttactccactcagcacagaatggctgcggggaaatataatcagacatgcccaaagaagtctg
cttagggtgaaacaaatacctttgaggtttaaatcaggaaggagagactgtttctacaggtgtcatacaaaaatcaatactgagccaaggattattggatcccatagaaccacg
gagacttggtagtgaaattcttccaaagcattccatccgtgcaggcagatggtgttcctaggtttggaaatataaatcccatgggaagtcagctgaagaccattttatgcatcact
catcctgttagagccctgtgctgttctgtctctctataagactggctgaaaccaccacggttgtttacaatttatgctctaactttggatcacctagttaatactctaactccgattatta
ccaaagacaatgttctataaaagatattgtctattagaaggatgcttctgcattattaataaaatcacagggaaaatgcaacattttggatgtcacatctgataagttggcagttaaa
gaacagaaacagcaataagactgcgtggctacattttcccagtgccaccaactgggatttaagctgactaacattttgaaagcccagcccatttcattcacagcaggatggca
gtgggtgactgatcgtacccgccagctggctaatgtcataattaacaaagttgctactacatctgcaaagtttaatcgaaaaatttgccagtgatatgatggccttgtcacagaga
tcacaagggacacggagttgacttttgctcaaggtggaacgacagagctgtgtaggcaagatgagaaattgcgaacttgttcggccaaaggattatctcctgagatgtgtaga
aatgaactcttgtcagagtttcaaaggaattaacgttttttggtaaaatgtgctaatgtaaagagtgagaagattagtgatacataatgaaagcaatgcattctttcacatggaaatg
ggaatataataaatgtcctgcaggtgattggggctgtagcaattttattgaagattaaagtttgattgacctttgttagggtgattaacccttctgaaattaaaagccagagctgaat
tattctgctcaatttaaatgcaaacatcgagcaggtcgtatgtcattcaaataataataagaagaaaatcttcctattttaattttcatttttgcacttctgagtggtatgctgctttagaa
ggaccatagctctcgcaaaagcattctggtggcagagaatatacaataacgttacagataattgggagcatataggaagagtcccaagcactactgcctgcattcgaaacatc
aaaaccaccatcccaacaaaagcacggagcatgaagccaatctctgtacactgaaaatgcatgctcccaatagaaaaacaacctagggatgtcctgaaggggttaggaata
aataacaatgacatctctattctagttcagtgccaaccaccgaaattgcatatcatatccatgttaaatctacatgatgtctcctggaaatgcgtgctttttggtacagttgcttatgaa
gcagtttctaaaagttgattatttctgatgtctggaatttcattctccatgatctctggcagtttctgaatatattcagtattttcattaagttcaaaaatgctgtaggagaagctaacga
attatttgcaatgccatttaatggtttagttttatttttctctgttaaattttctaagccttggtgaaaagttgtgtattcacatttcttcaaaaaactgtttccatttttcatatcatgttaattttg
tttagttctaagtcagtgaatatttttagtcttaggtcccaccacatagtgaatgtgtctgccttttatgtatctgtgaactattaaaatgataataaaggttctcttccttccat
Mouse-RMST
(SEQ ID NO: 136)
aggtacctatattctgcacgggcttctgggcaggcagcactaacactcaggagaaggtctgcctctgtctgcgccttcctggttcaatgcaggcagatgagtgtcac
agatgtaactgaggcactgaataatcaacatatcggagccaattcccaggacggcaagagggggaaccggcagagatgactgaccctggaggacaagagcattcttctga
caagttttagcatggactatcctcaggaaaatgagatccaggaagagtgacgccctcctcgaactgaaagatactcaggttgatggagtcagggacgaagtggcttatcaac
aacacaggatgtctgcagggaacagaacttattcagacatggcccagattgtctgcccgtggggactgagggacctctgagggttacatcaggaaggaaggagaggctgtt
tctacaggtgccacagaaaaatcgatgctgagcctcggattattggatcccatagaaccgtggagacttggcagtgaaattcttccaaaggcaggcagaggttttcctggtttg
ggaagtataaatcccgtgggaagtcaactggaggacattttacgcgtcactcatcctattatgagcctgctgctcctccgtctcttgacaaggccaccaactagaagggcaca
gaattgttcataaaacagctgaatgtgaatgtctttggattcccatttaatgagggtgtcttaatgagatgactgaagagtacaaccgccaaggctgagatgatgcaaaggctac
gtgtgggtacagttcccggtgcctgtgcacaccattcattggcagctaaaggacaggagcagcaataaggcttcctggctacattttcatggtgccaccaactgcgagttaag
ctgacaaacattttgagagctcggcccatttcattcccagcaggatggcaggagttaactaaaacagaataaactaaaaattgtgccttcattgcacatttgcactgattatcagc
actgaaaatggctaagttctgcttatcaagagcgggtgactgattgtccctgccagctggctaatatcataattagcaaagttgctaccacatctgcaaagtttaatcgaaaaattt
accagtggtatgatggcattgtcaccgcgatcctgagggaatcagagtcgacttctgcttcaactggaatggcaaggctgtagagctgagatgagaaactgtaaacttgctca
gccaaaggttatgcttcacatcacccaccagggaggatcatctctcatgaaaactttcaaatttgattttgaaaatttattcagaaaaatttaagccacgctctttcccaacacagg
aaataatatgctccagagaatcctctaattcaccgaaaatggacttggataacatttgggttctcatactaccacagtcctcttgagaaggacagccaggcttgggtggcagtg
gtgaggaaggttcctttctgtcaagggagctgatgccttaaagaagctatctccagataaagggagatctaaggaaggaagccaacgaaagacaccatgtaccagaaagcc
aagcaggattttgacatttataaccataatctctataaacaagatgaattatggctggggattcagttagtaaatcaacgaaaataaatcctggtaaggtaatgatacaattttatca
ttttttgttgaaatctaacagtgtatagtatgctttgctgcaatcaaagacctgcagtattttgtgtgctaaactccaatgacccatcatttattttctctttattccacctacttacctgtga
taataaccaaatagcctcacatttcccctttcaaagttgtatctcaagaggaaagctcactgaccccacacattagccctccagtagctccagctgagaagtactctctcctccat
ttttttcagtgcataattcaaagataacccaaataagaacagaaagttgtgtgtcatagtcactggcatctatccggtgcttctgaaccatgaaccacatgacctgattaaagaggt
tgctgtgagtgtgttgtactcttcgtgaactaaattccatgatgacagaaaacaaacctgaaaacaagagttatttcacaactctagagctccacagagtcggctgcaattggttg
agttgaggtgaattttcagtggacctggatcagtgtagaagggcaggaacattggcccagcacagccccaggctgtgcagcattcaatgtgacttgggactggccttcctgg
ctctgccttgccggtgaaacatggattggaagctttgaatgttggaagaaataaacaagtcttggttggagtcccagtttcaataaccacatattttcaaacctagacaaatattct
ggtctcatctcctcatttggattacttcaatcctcggagagaacggaataggactctgtatttctttggatgttcaacacagtgtttaactaatgggtgctatttgaaatattatcgtct
attatcattgctattctaataaggatgatacttgctataaatattattgagaaaaaaaacttggaagtgttcttgagcaacataaacagtacaaaatgacagatattaaacaccgttct
tttttgcacgtaaaactatacaaataaaaatatagaaaagcttgc
human-tuna
(SEQ ID NO: 137)
gtgctgctggcgcccggcccccgcggggtgcagctctgcgcgttctcatgctgtctctctctctttccctccgcgctgcctctccgaggtcctcccgccgagcccc
ggcgcggggcatgaggagcccccgggtgccgcccagagaccagcaggctgcgcgcacacctagccagcggcagacggggacatgagcagcgcgcacggggtccc
gcgcccggcggccagccctatccggcggcggccagcgggtcaacgctgcccgggagaatgaggcaggagccggcggcagcctcctttttttccttctcctcgccttcctg
cggctccggcgctccgggtccgggccgggctgcggctctgctgcgtgccccgcgcgcccctcaaccgcctccggatgcgcttctcgatctcagcccgctgtcgccttctct
ctgaccctggtcccttccactgcaggctgttgctccggtgtctctgggctgctccaccacgtggtgcctgcccatcatggcatccaccaccaggtggcctttttcttggttagcct
ggcaaggaagataaagacatttgcaaccaagatggtaatcactagtgaaaatgatgaagacagaggaggtcaagaaaaagaaagtaaagaggagagtgtcttggcaatgc
tggggattatcgggaccattctgaacctgattgtgatcatatttgtctacatatacaccaccctgtgaatggcccagagcgtcctcagaggcctcagaatggccaaagacggaa
gtcctgcgtgtcggcgcatcactgaccagaccctgcgagaacaagcaggcttgacccgcacataccacccaatcaaatgcaccttcaaactttacaaaaggtcacacaaata
gaccgatcctgctgcagggagcagacactaaagcacaatgattccaacaaaactcattcacagcactaggaactcaacgtctttggcagggggcccagaagaatgcttgga
agaccagcctctgacaccatcagtgagcggatgggtgcagaaattcattattccagatcgctgacagatatcacatatttgaaaagatgaatagggcggacatggctcagatg
tgtgtctcccaggacaagtgtttcatcttcacttgacgagctatttagtggaaaaaccacaggcgcagccctttgacaggcatcccattcatcaaaagtgtctaactatttgatact
ggggagataacttatttttttttttcattggcttgacatgtgtatctgttcatgtcaaggtttataaatatatatttttaataaatgtgctctattttttagcatgaaccaaatacttggaga
ggcactcccagatccatagagctttccttagttttatctgctttgtcccctcctcccccaactacagatgttctgttgtggagccattctagtccttttgtctcatcttgagtcttttacctt
gcgcttttgttctctctctctcctctctctctgcctctttggtctgaaggacattttcccatactgtcagccatggttttgggtgcatgttttaagattgtccattgagtggctttttgttgtt
atctcggagatataaaatgattgtgggcatgcagaccttagatgcaccctatctttactgagaattatgcatgaataagggctgagtgatagatcagcttaaaattaaaaggacta
cctttgaggaagaagagcgtggctatatttgcagatgaacttttgaacagaatattcagcttcttaccggcagcgttattgtttcattcttgtgaccattcgtttatcagattttgatttt
agcggtcatgtaccgcgagagttgggaagaacaagggggaaagctcgggattaggtgcattactccttcctttgcaagatacctgggatcctcctcaaaagcgggtggggt
ataaatgacacaagaactcccccaggagatctcatggtgattcaggctgtgaggacagccctgtgacaggtgacttttcagggacatgaggaggggatttaatgattgcccta
aaggacttctgtatttttaaagcccctggtttacacccacatgaagctatttcctctctggcagggatggttgcataaaaacaaattagctcccttctggctccctgaaatgggccc
ttgcctggctacagtggcatggccttaaagagagggttagtattccttctgccattgccagctgtattagtctgttttcacactgctgaaaaagacatccccaagactgggcaattt
acaaagaaagaggtttattggacttacagttccacgtggctgaggaggcctcacaattacggtggaaggtgaaaggcacgtctcacatggtggcagacaagagaagtgaac
atgtgcaaggagactcccatttttaaaacagatctcgtgagactttttcactatcatgcaaacagcatgggaaacctgcccccgtgattcagttacctcccaccgggtccctccc
acaacacatgggtattcaagatgagatttgggtggggttacagccaaactctatcaccagccttgcccctgggcagaagcagcagcagtctgcctggctggattcaaatgatt
ctgaggcttctatagtctatgcctgcagatctctccctcacccatgctatagtgtctgaaattccaccattagagagtcatttcttgggctctgttaaatggaccaggctcttttataa
agaaaatgcccctgagcagctggctctggcattgatttatgatatcttctcttccctgccagaaggaaggaagctaaggtgcatgtagggcgtactgtgtgcccaggcactgtg
ccagatgctttggatacttggagtcattgaattcttgtagtaaccctgtgagagagggagtcttttctccacattgtagaagaaggaaaaagggctcagagaggtcaagaaatgt
ccctgagatcacatggcttctagtggagtcaagatccaaacccaatgtgtctgattccttagcccttgggggtccggaggctgctgaacaagaaaggaggtggagaggaga
gaaagctgcaggcataccaccgcacacccttctccctcccctgtaaaaacaaccctgggaactccctggacactagcagaatatcatacactaaggataagggatgagagg
aggctggttagaaataaagcagtgtcagggggaaggagctactcagtaggctctgtgtgattctagaaagactgtatgaaaattctgaacagtgaacagaataaacaataaa
ggtgcaatggaaaaaaataaaaaaaaaaaaaaaaaa
mouse-tuna
(SEQ ID NO: 138)
tcattcccctccttatccctcctcttctctccccttccctccccgcccttcccctctgccgcagcccagctcctagcgcgtcagcgtgggcttcccggagcgagtcgct
aggtaacagggctagctccgcagacccgggccagggaagcccgcgcgcgtcgtcagcatctgcgccgaggggggggaacgcactgacactcccctatcggagcctg
cagacccttcagctggttctcagtcccctccctggatgccttaagggtggctattgccaccgcggagttgcccctcccctgcggggaccccgacgtcgccgcccccttctcct
cccacgctggtccgcaccactctccgcaccgccagccaccctccgagatcttcccgccgagcccaggcgtggggcatgaggagcccctgggtgtcgcctgcagacaacc
cagcctcgcgcacacctcgccagtcgctgacagggacatgaacagcatgattgggttccggtgtccggcggccagcctcagccggaggcggccagtgggtctgcgctgt
ttgggagaatgaggcaggaacccacggcaacctctctctcctccttctcctcgccttcctctggctcccgctccccggacccgggccgggctgtggctctgctgcgtgccct
gcgcgccccccacctgcctccggatgctcttctcggttagcctggagaggaagataaagacatttgcaaccaagatggtaatcacgagtggaaacgatgaagaccgggga
ggccaagagaaagagagcaaagaggagagcgtgttggccatgctgggcatcatcgggaccatcctgaacctgatcgttatcatctttgtatacatatataccactctatgaaa
ggccccattcagacctcggtggcctcaggatggccacgagacaagggtcctgtgcatccgtctatcagcgaccacacactgcggggggaccagcaggtttgaagaccac
acaatgctccatcaatgcaccttcgaactttctgacgggtcacctgatggacccgtccttctgtgaggagcagacactctgaagcacaatgaatccagtgggactccttcagg
ccacaaggaactcccacaggggacccagcgggatgctggaaaggccagccttctgcaccggcggtgagcacgagggtgaggaagtgcttcaccctggagtgctgacca
catctcacactggaaagaggatgcgtggggtagtcgcagctccgctgacctcctcccaggactggcaagataaataagctctaggtgcggttcgttgacaggcattgcattc
atcaaatgtttccagctgtttgatatgggagataacttatttttcctttttcattggcttgatatgtgtatctgttcatatcgaggtttataaatctatatttttaataaatgtgctctatttttta
gcatgaaccaaatatttggagaagcacttctggacccatagctcttcttggttttaatcgcttttgctccatcagctgcaagacgtctttgtctgtagtcatccggttgcctcagctca
tctcaagccttgtcgtacctcaggttcttcttccttcccatccctgcccctcccttctctcctctctctttcccctgtctctcccttccatctcttaattcccctatccttccccatctcccc
accccccatctcctctctcccctcctcctcacctcttgtctagaggacattttcccataataccagctgtggtcttggctggtgtgtgcttcaagattgtcgagcggcttttcattgttc
tgtcagagatataaaccgattgtgggcatgcagaccttagatgcaccctgtctttattgagaattatgcatgaacaagggctgagtggcaggccagcatctccagtttttaaagg
gactaccaaggaggaagatgagcagccctgcctgcaggccacccttgggacagagcagtcaccagctcaagtgcagtattgcttatttgctcctgtaaccatccaattatcag
attctgatttatttatttatttatttatttatttatttatttatttatttatttttgtgaccatgagagatggaaacggggcgagctcgatgttcagtgcaccagccgtcatcccttccttggtg
agctgcctggggaggtggagggtgggatctcttcagaagtcagtgggatatgagggaagtgagacctccccgctgtgagaatgcacacacagttcctgctgtaaaaacact
gctgggacgagtgacccttcaagggcattgggaggggacttaatggtctctgttaaggagccctgcacattttaaagctgtttttccactcacacaaagctctttttttctctgagg
ggatggttgaataaatacaaatttgccctgctctctggctccgtaacacagccccaacctggatgcagtgtacagctttaaggagagggtccgcattcattctgccaagggca
gaagtggcagcagccccccctttggattcagatgacactaagggggggcatgtctcagcttgcccccttccttaaatggcaccatgaaggagtcatttcttgggcttcactaag
ggaccagtctcttttataaatcaaatacccatgagcacctggctctgcccagagatgtctcctcttccccaccagaaggacagataccgtgtacacaagtaaccaggctttcata
gttgctggtgagagggagagtctttctctctcacctgtgtggtagaaggaagaagaaaggacagctcggaccaggaatcttccctgaggtttcctggcacccagtggagtca
gattctagcctcacctggccagttccttatcccagcgaggacttggatgctgttgtgggatgctgatgaggatagtgactgtggctggaaagcctgtcgctttgctgaggatca
acccagcacacacacacacacacacacacacacacacacaccacaccacaccacaccacaacaggaaacaaccttagaactccttggacactggcagaatttgacacaa
ataaaagacagtagagaggctggttaggaaggtgggaggcaccccggggcgagcactacctaccaggctgtgtgattcttgacaaactgtaattctgcacagtggactaaa
caaacaataaaggtgcagtgggggaaactattggtgtcgccaacttcctcgtgagcccgtgtgtaattgtttgatttaataaaacttgaccgtggctgtaaaaaaaaaaaaaaaaaaaaa
human-Linc-Brn1b
(SEQ ID NO: 139)
gcgagtgcgtccccaccagcctggccgccgcgggaactcggggaccgggaggggggggttagcgggaggaaagggtaagctgaagaggggctgggaa
cgggagggaggctcggggcgaacgggccgtcgcgttcatttccataagaaagctgaaatcgcagttggaagcaggatttctcgcccctgcagcccttcccccatacccggt
tttctgcaagaagcgggctccagagctcagagcccgaagtgtgtgctctggtctttgatgatgttgataaaatgtggtttcaaatgttgtcccgaagaggccggtttcctgtctgt
atgtggaatggaaggaccgtgccggagttgtgttctcaccacccactttgtcggcggaggagaaacactgctgcctgatgcttgcactgctgcactgttcttgcacctgctcct
agccctgaactcgggccctgagctcctcactgccgcccgctctgcgctgagccccactcctgagaccgcgaggtgccagagcagcgcctctcccaccaactcctagacgc
ctgtaggagctgcgccggagccttcagaaccccatgggggtgtgggggtgaagttggaggtcatccgggtgatttttttcttactttttcctaattgttctcctgcgttgatttcaac
tcttccagtctcccggtgctggggcgccaatcctttcccaacaattcctacccaccagctggaggtcggggcccagggttaccccagagttctatggtggcagggggcgcc
gagtagaggggcgggaggagctgggtcctcgggaaccaaaggggacctcagcgggggcggagtgcgcagccttggctgctgggaaatgcccaactagccgctgtga
gtgcccacagcgcgtcgtgggggcgtccgttctgcagctcgggtgactcagacgccctgccaaccccaccagctcccggccttctcgaacgctcttctgatcgcccagcgg
ggagacttcagggcctgcctcgtcaggccagattgcagcccgcgaccttcccggattaaccaagggcggggcctgttgcccagcaaggctgctgcctaactgagcctccc
ggggaacagccatgaccaaagccccgcgggcccaggcgcgggcacctacccctacccgctcaggcctgtgggagcgcaggggcagaggccgagacgctggacttcc
agccccacacctctgacaacccgggcacgtctgcggagcccgggggaacagtcagcgggctctgcgtcccgggagctggggacacgcctgcaaggtgtgtgatccttta
aagatccttagtactagttttgtagggagcctttgttatagctttgtcagcctgcgcgccttcagcagctccccctcccgggcccctcggttccctccgggctcgcccccagcgg
agagggcccggcgcaggggatctgcccaggatcaactgccctcttccagatccccttctgcgagattcttttcctttggttacaatctcccctccgtgcctggggcagataaac
acggttcgccccgcctgcagtattggggcgatgctgtcctggaagaacgggctttctgaatgtgacctcgggatgctgcagcggcgcaatgctgctactgctgaatgagtag
ctctcctgccaccgcagggcgactgcggtgggcacagcagggcgcacagaggagggggtaatctgcccacccaggctaaggatttgagggttctgacaaaaaagagct
ggttgaggatgacacagttgtgaactaacctggctcctactaccccacagggcagccaggtcccagtagtcttgggtaccactgcccccaccataccaccattgtaaagtatg
gactcctgcttagccaacgtgcagctctaggcaggatctctgaaggatgctctggcacacagatggtgcaggtatgactttacctgcacacaagcttacatgtgccctgtcata
tatggctacacgtttccccatttccccacagtcttcctatttaactggtcttcaatcctcctttctagggctttccgcctgcacacagtcatttggagaaaatcaagtgagtgcacttct
ccccacagaggattctgtatgttatgtaacacactcacactgggattcctcaagttaccattttgccccaactcagccagcacttggttttctggggggggtcaggatgatgca
aatggaggtggaagcttcattccctgggggcttttattcccctccttgaagccccagttcaaggcccaggaacaaaggggaaaggtaactgatctctcttttggtctttctctccc
ctcacttccaccccttcacctgcaggccctcctctttgataaatagaggaaagcccgtgggatcaggtacatatgtaaatactgctgacctcggccgcccctcctcaccgaaaa
gcagaaagcccgtttcaggtgcaggctgctccgtgcctttgagtgtccaccacttctattcccccagactgcttgggactcaaggccactcacttaatacccgtctcttgttaaaa
acattcaggacaactattaaatagttattggaagagttaaagaccttctttcaggagtgaaacgtttttaaagctgtgcttggcataagtctttaaggcgtacaaagtagaatcagt
agacttgcctgagcgattttgaaatttgaaagatttctccccatgaaatggtaatttaaaatagtttgtagatgttgttttgttctgtgatggctttttcaccattgtcacctaaagattct
caagaccaagctactaagacatttcatttggttagtttctgggtgatactgtgctttctgattagacctatttaatttatttttaactctgcaggagagttttcaaccttggaaatgtaagt
gaaacacagactcaccctccttctgagaccttgataggggcagaaaggaacaaatctggctagcccccctcacacctggaagtttccctgtctaaacctgggtacaatgagc
gtggtgggttttgtcaccttttattttattgctggctcaaggaaaattctttgaaatgaacatctattctatttcagtgtgcaatgttccagccgacttaatgggcttgccttatgcggag
aggcagtcctgcaggacagagctaatgtgtggtctcccaggaagctcatcagaacacctcttgtcccttttcttt
mouse-Linc-Brn1b
(SEQ ID NO: 140)
ctaggcgactggtgtggattggtgttgatgagatgtggcttccagcgttgcccgactcagacctgtttccagtctggatggagcggaccgactgtgtgggatgggg
tgagaggaacaagtgaaactcaacagctaaggagaaaatgaagaactagtgctacgttttccacgttctttttgctccggggttgacatggcatttggcctttctacctggagaa
aatcaagagtatgtaactttccctgcagatggtctgtctgtaagagctgacacaggaacctttgggagccagtatctattttgtttccgactgcaaagttccagccaatgatttgga
ggggggccggcaccttctgctgggagctagtctgggagggaacaactcattcctgagttctctttcgcaaacatatctcttgtctattaaaataatagtaataaaaataagaataa
taattaaaa
Mouse-Dali
(SEQ ID NO: 141)
tttgttttttaaatcaagattatagattttggggaggtgactcaggagttaagagcatttgcaactctccaaggaggcttggattcagttcctagtacccacatggtggct
cacaatcatctataacttgagtttcaggaaatccaatactctcttctgacctctacataaaccaggcatgcacatgatgcacataaatattgtgcaggcaaacaccgtcccccaga
cactcaaaatacataaaataaaaataagtattttttaaaagttggaatttggtatgattagctttatagctttagctatttatattagttttatggcaacttgacactacccagagtcatttt
ggaagagggaacctcagttgagaaaaatcctccaccatattgatctatgagcaagccagtaagcagcactccttcatggcctctgcatcagctcctgcttccaggttcctgccc
tggttgagttcctgccttgactttccctcagtgatagagtgttcttgggagtgtagatgaagtaaaccctcgcctcctaaactgcccttggtcatgatcttttatcatagcaatggaa
cccctacttaagacaattatctttgaagcacatattttgacatttaggcatatcaataaatgattgatggttagtttcttaaaatgccccttgaccacaaagacagtgaattttctccctt
gctccctagcatagattataggtctgagccatcagatctggcttgaggttcaaagcttgtgaccacctgttccaaggacaaatgagctgcctagaactcttccactggcctgcct
gtagccgggaatggtggagttccctgtgactgtgggattctatatgagctactgatgagacataagcaagtaaggaaagttgatcttgggatcaacactcatcaagaataagat
gggtcccccactctctagctgtgcttccagacaggtttgcttcagtgtggcttcagcaaagccttcctagtggtctacagtagagaatgcaagagtccatccctagcctgaaca
ctgcacacagacaccaattatgctaaaatatttaatttcattccataaataattaagcctaaataataatacctagatataacttggaacttgtgacaatctacctgcctctgtctctgt
gtaccaggattataggcctgggcacacattcttcttcaggactgattataatacattttgtatattgatttaaaaaaatgatgcttaggtagttatggcctgtgagtaattctataaagt
agactaagtggatatcatcagtctatactctaagacattcctttaatttttttaaatttttttttaaagttctactttttaaagattcctagagagccagatgggtgatgaaaagccaggtt
tcacatgtaacactaatattgggggaaaaaaaacctgacgctagaaattatttagttattctgaaagctgtcacttcttgaaagcagagtcagaattagaaaagaattaacagtgt
atcgttgtgttctcacatttccaatgtggctggtgaatcatgaagcaaaaatttcctcaaaactggagccccagagtcttagtctgagcccatcctcctgcctgagaatctccaaat
tgcctagtgaaggaaaagctatgtccatggtcccacctgttccaaagtccctttggaaatctaggcagaagtaggtttgctagagtcttacttatgacaacctgggaaacaaag
aaggctcttcatacaagggtgtgtatccacacaagaggcagcatcctgaaggaaaccttcaggagcctccgatcctgcagttagctgactgtttcctgccctccttggtccaac
tcgttatggccccaggtgttccttcttgctgctgaactgctgtctctaccagctgagcccactcctgtggtctgcactcactgccctggctgcaggttgaacagggtcaggctact
tctcagatgcttcccaccagccgcagtgaactccattttgacatgaagccatttaaatggaaatccagttttacaaagctcaagtgactggtaatgaagatgtgaatgatgcttat
gaggtttttatttaattttaaatcaattgtgtattctaattcattaataaaggggaaaggaaagaatacaggaaaaaaaaaaaaaaaaa
human-miat
(SEQ ID NO: 142)
cagctacaaagacgacgccggctgcgctcgcgggaccagagggagggaggctgcggacgagtgcggggaggagtttgagatgggaggcgcttgcagggg
ctcccccagttctgcgctgtgagccggggcacaaagagccctctgcactagcgccgcaggaccgcggacccgagttggaggcatctgtccacccatgtggttccagacac
gttcatgtggccaccatgaccccgtcggcatcacaggggtaaccaaggtccacagaacgaggggaaagggtcactgtggatcaagccaggctcctctgagcagtccagg
gtctatttacagagagcagcaagttcctgggacagaggagacagaagggaatttccaggttcttcaggacgttcacaaccacactgagaagcatctttgcagataagtatttaa
acttaccagcccatagcctaccagcctgccctcttctggtctgtgcaggaaagtgtagtatcctagaatgccaaagtgggaggggaaatgggtgatgtagctcattctcttttct
acctctatggaagaaagaaagagcctgtccccattttgtggggtcccagaaagggtgattttcacacttcacatttggcgttagggctagtatttcacaaacattaccgtctggaa
cttttgaaggctgagttaaatgacttcatcttgtgtacttgtagaactgctattagaaggaggctggggactccctgatgatgtgaaccagcccttctcttctcagtttcagcacttt
gtgatcatatttataaaccttggacagggctccttggccttagagttaagtgaagagttaacactgtgactttagatgcatttttctcagtgtaagtgtctgagctcatctccagttct
ggactcgtgcccccactcccgggtgctgacagcaagctctgaagatatgctgagagagctccctgactctcctgactcaggtctgccagttgatgaagacagcacagttgtta
ccaaaaacaaaacctggcagatggtcctaggtcaggattagtggtcattctctggtcttctccagactggtgacacagggtggtcttaaaagagtcctagtccagcctctggct
ccccgaatttcctctggtgcatggggcatcaggaaagggcaggggagaaggagggggagcagaaaaataactgaggctgggcagaagggtcccttccatttcccatcca
ttgcttcaaacaatgggtgattaccgtgcaccttgagtgaatgtcaaggcaggaagggttccagggccacccagtgtggcaggagcagagctgggatcagaattctggattc
ctgactctccgttcagctgctattcagctatcaccaagctgctgagtttgtcctactttctcctgggtcacattctgctgttgttgggaaacagggagtgagtgaagggaacctgg
gaacccttctcccctcaccttttaaggatgttctggggtggaataaagagaggtggaggggggggcctgggcccagcctccctggctcctggtccttctttcttcagcaggg
agttctggcaaggcctgggcctggggagggggcctgggtgcagttttctcctgcggtgtggttggctcttttgtcttccaggatgggcctgtggggtctgtgtggggaaggaa
ggagctgggagcctgggctgcctgtgtgtcggctggatgctgccagcaccggcatggaccaagcaacttctctgagccagatgctggggacgcagaggtgaacccagag
acatgatcccatctgctcagatcttcatgtcagaacacgctttattacagtctcggcaagggggtggctccatctgggccgcatctccaggactcctggcctggacttctctttg
gacctcgggtgaccttggattctggccacagtgacttagcggtcagactccggtggttttctagcccgctgggtgactccctgaagatctcatcctcttcacacctgttgggatg
gagcctcccatggataggaactcttcctggttgatgctcagcagatgactgatctggctctggaggacagctccaggggtatgagggaggcctggcagcccactactccctc
ctcacctgctcgggccctgcctgctgggccctattgtctgggtctgggggccctttcctgccccatgtgaacttgattcctcctcgcgtttctgccggtgaccagacagagcctg
gcctgggccaagtccccactgaccacactttccccaaggctgggtctcaaaggaccctacaggatggggacaggaacaaggatgggagtcggggaggggctgtcaggc
agagggcagatgcagctcctcgggtgccgctggctggagtcctttgtctccatttgctcagtgcgtaattgtcattttgtgggggacttccttggtggggcttttccatgcttttcct
ctccctgatggttacaaagaggagacagtcaggatctgggaggagctggagccttccccctgtgctgttgaactctgggaggggcagccagaggggctgagagtgcacc
atcctgaaaagagagggtctttaagagataagatttaggctagggaaattcactagaaagagacaggtggccctgggttttttttcctgccatccaaagggaaccagtcttacc
caaaggcttaaggggtcaccttagcccagtctcaggacactcttctccaatcagatcgctgcttctagttgcactgctcctggattctgttcttggggaacagaaggtggcagg
acaggccccacaatctgcaattccagctccctccagaggagacagtcccagcgtttgcaagagagcagcttgtggcctcccttaggcaagtttaaaagccagatgtcctttttc
ccagaatgcggagtggtgtgtactattcatcagcttgggctgccacagcaccatgccagactggatggcttaaacatcaggaatttattttctgccagttctggaggctggaaa
gtccagatcaaggtgttaagacttggtttctggtgaggcctctctccttggcttgcaggtggcaccttctcactatgtcctcacatggccttttctctgtggagagggacagagag
catgagcaggctctggtgtctcctcctcttcttataaagacactaatatcaccatattagggcttaaacctatgacctcatttaaccttaaccccttaaaggtcccatctccaaaaac
agtcacatagcaggctactgcttcaacatatgcatttgggggaggggacaccattcagttcttaacagggtggtcaccgcaaacatggaaagtcagagccttctccccttcag
aattcccgcccccacccagggatggggaggaggagcagagaggtatgggaagcagacacggagagtggcaggtaccatgctggggggctcaggagtgcttcggagg
acatatggaactggcagggctcagtgcagggaggcggaggccctgggagagccgtgtcctgagaagggcctgggctacaaccctgggcaagttacttcacctctgagcc
tccgatgctctgtgaaatggaaggaatgtgcttgcctgtcaggtgcagggagaattcagtgagattgtgtgcctagcacagtgcctgactcccagtaggtgctcagcaaatgc
tcccatccatctgggagtatagacttagggtttatctatttttttttttttttggctctctggactttaaaactcagcatcttctgaaccagaggcatttctgattagcccttccctacctatt
ttcctagtatcactctttaatcagcttggggaggtggcagcatttcatggcctccgtagtaactcacaatgcttcctggggtatttaaattctactctctcatcagcactgagcacct
actttgggccctttcccgtgctagccatttgggggaaatataggtgaagagtggctggggtttgaacctttaggatttcacagccatgctggatgggataaaaccagctcacat
ggagaatcagagaatgggacagtgccacaaaacagtctactgaggcacaagttctgagtgcctgggaagtggtagagagaccccagagagggctgctgcagtctggaac
agcttcctagcaagctgtaccaaacagggccttgaaggatgagaaagatctggctgagatgatacccgaccctctagggaaattcttaaagtaacttctaggaaatgtcattgc
tccttaaaaaaaaaaaaaaaaaaaaaaaagcaggttctaattcaaattccgctgcactactgaagtagtgcaggacttatcagaacctttaaaatgctaatggcaatttcaaatac
tcagggacagggcataggatgaagtgttccttgacttttatggccgtagaatctttttctcatggaacatcgtttagaactgtgggagatgctaggctggggtttctatgggaaaa
gtacagcagtgatagaagatggcataattagggtacacttaggtgctttgtagcattggctgaaaaggaagcagaccggggggcgtgggggagtctcaacttggttccagg
agccaggccagcttgatgcgttttttttttttctaatggtgggagggcaggccaatagttggagaaactagaggcctgacagtcggtgatggaagagatgtctactaagcacgg
ttgtcagatcttttgcggagagatgcgggtgtgcgtgtgtctgtcagagtgtaacaggatccagccctctgggaaggggctgaccagaaccaggcagggggtttctgtctag
gacaagtagggtccattttcaggggagctgaaatatttgcagggggtgctctgagacccacagccaataaatagggaagcaacatgcttttgggcagggtccatgtggttag
ggttgattttgatggggctggggggcttagagttagaccccctgccctggtcaggccccactccctggctggtggggggggaggcctctgaagctgcagctggaggggc
cggggcagtgttgcatggtgtgattaacctactaacccagggctcagccgggacgctgtggcgtgtgacgaggtgcctggccttctgccaaccaactaacccactaaccag
gcccgagcatgccctactaaccacccccccgcagccagatcctactaactgtgcagctcatgggcctttcaaggggctgggatatgggggcagccaaggctccagaagg
ctctgcctatgggctgggatctttgtgcagacccacgttccaggacagagaatgcaaataagactcaccaactctgccacttcctgttgagtgagtttgagtaagtcacctcact
tctctcaacctcagtttcctcagtggtagaatgcgagttctgataccttacaggttgcagtgaggattcccgtcaatataagggtctcagcacatggcagacacctaaaaaaaaa
aaaagtatgaggatggaggaaggcccctcaaacctggttgaacacgtctgctgcctaggccctggcctctctgggtgttctgcacacagatccttctgcctggggcaagaca
cttggctttgaatccatgtcacttccccacattcttcctccgtctcagctttcttttctgtaggcagctcttttgttcgattactaacccgtctaacccctggggtgcctcaactgcacc
ccgatcatcccttaacaaactaacggacctgcgtgcgttcttccttttctcttagcgactcctgtgtgtgtctgctgaggtgccctgtccgctggtgctgtgctctgacttactaacc
cagcccctactaaccctgttttctcttcttactaaccccagccctgccgagctctgggctccccccgggggctggtccccctccttttggcaagcagatgacctggggctactg
gccctgtagacagatgtcccactttgctgccccatattggctgtaagatcagagtccactgggccaggtctaaggcaggggatggccctattaacaagactcagaggaggaa
gaggtggtcctgtggatgtgggaggctggactctgagtatgacatctctcctatgtgcagaagtctggttgccactgggagtaggtgggaccagggaaatctctgggacgtg
agtgtggaggcctgttggtctagactctagactgtggagctctgagcttttgtgtcctctggaaggaagctggggaagaatcctctccattgttaagtgacggggatagaagct
gtcctgcacaggaagtcacgaggggggcgtatcccacgaggaaggcaggagggggcgtgcccctcaccggaaattagcagaggggcgtgtcccacaccggaagtca
gaaagcggagcctttcttacaccggaagtcaatgaagcgggtctttcctacgctaaaaaccactgagtggagtatttagtacacaggaagtcggccagagaaacatttctcat
atttgaaggccggaaagagggacatttctgacaccggaagtcagtgagaggactctttcccacacaggaagtcagctagagagccgtctcccctctctggagccgagaga
ggccggtttcccccaccgtaagtagacgtggggccgtgaccggaagtccttgggaaagatccgtcccattcccggaagctagagggcgttagttgtcgggttgaaaaggg
gtgtggggaggggaagcagctttaccccgggctcggagtttgcaggagagagaagtggggagcaagaagtgaacctcaggggctcacagggttcccgcagatgctcag
gccggccaggaatgcatctctggctctctgttcccacggacgtcactgcctcagccagcctcccccagagcccgccagccgctaagccggggccacacctgggggtgatt
tcatgcctcacctccagtaggcaccttggtttctttgggctaatctctggctcccttgcgctaactcttgctctcacccagctaatccctgcctcaccctgactgccccaggggct
gaccactaacaaccaacctggccctgtctgggggttccaggctcctggcctggccctgaccggttcttaattaacctttccttcaccttgactaactcctgccttcctggtctgttc
ctttcagcagaaactaatggtttgtggatttttttctgactaacaacaggtctaacattcctcgttactgttaacagcttggatgtcggcatggctgggaaggggctaacacagcttt
gaacttggctaacacaggtttgaacttggctaacacaggtttgaacttgactaacacagggaaaagcatagctaacaattttgggcgtggtggctgctctgagtcagaacaatc
agaagtcggtaaagatggtagttttctaaaggaggtgccagggctctggtgtggaccaggcctgatggagcagtggtacccaccaaggtggggtcagaagtatagccagtc
ttgcaaggttttggccattgggcatatcttcactcctcatagtctgcatttggtttcagttcttaaaaaaatatagccttatagctacagtagtttgcacaagtagatgcagctcttata
aaccttaaaatacctgtctggtgtctacagagcatatgccattttgtatagagtcacccttccccaggccagggcctagagtcttcattttggggactttgtgttttggaagttctag
gacataaagctttagatcagagtattcagaagggttataactccgtcagtcatttacatgttagtagtataaattatctgagcttcctgttctaacttttagctcttctagcataaactct
tctgtgcaatttagctgcaccgaggaaacgggagtttttctggaagggacttttgatctctttagactgagggaacgtcctttgggagtagaggggcagggagcatacgcaag
ggattccaggtgcaggtaaaaggtggcactagttcaaggttttgctgactcagtctggtagtcagagtctgcaggagaagacagttcaaggcagggcctggaggattggatc
agtttagggacaggtcaaaggctggcttacagaccttagaggcaggttgcttgggtcgttgaatgctagtctggtgctgagagcccttttctctggcaactgtggactcagagc
taaccaattgtagttggcagtgggggtgaagggtgatccagaggcctgagctgcagagggcacaagagagaaaagatgtcttagaaagagctttgagaacatgccttggct
gctggcagggaccttggatggggtagtctacacccggaagtgcctgcctgccatcctctagtggctgccttgctccatttcactcaaagcaggaagctcacacctcctattcct
gaaactcctctttgtttaactgcaaagacttgatgctgctaaggatctactatgtgccaggcactgctctgggcgctgggacctgcacctgggctttttcgtcatggtgcttttata
gcctagtgggagagttggtgaagtagatagtgattcagtgagatgggtgttatgattggtcaggggtctgtgggagcaccaaggagacagacaagattgatgtgcacctact
ctgtgccaggcgtgtgccaggcattggggatgtagtggtagttaaacaccatttggtcttcaggagctttaattctagtgtgttgggtgcaggggggtggaatggggacagag
agacacctaatccaccctgtggtggctttctggagagggaggcatctaagctgagctgtggctgggtggagtgtgggtggggatgagttccgggcagcgagagtggtgga
caccagtttctggggatcagagaggatccaaagaggttctggaaggttcatgtggaatgtagcaagagataggagacatggacatggtgccgggtctggttgccaagaagt
ttagattttatccttaggccttggggagcgacggatatgatctgagaaagggagttagtggatttgagttttaggctggccatttggcttttccagcccaggtggaactcagagg
agtttgcaatggcctctggccacattttagacaactgagcagaactttttgaaactaggaagaccctttggtccatcttttgataaacagaatccatacatgtctaccccagttgga
agtatctctgcaatgactggaaagtaaagaggaccaaggtgaaaataaaggctcggaaggggagcaatcttgaaaacatgtcatcccatggtggtgggaagtccctggaga
agatcaggggaaacacagtcataggctgcaagtctataagataattccattggggagggagcccatttgtcatgcatggctgcaaggggcagatacaagtgtggagtaagct
tgcaagagctgatcctggtcccagagagggaaaaatatgccttggtgggtaatgaaccttttgttcccagaggcagaaggattgggactaggccaacatagagattggcgat
ggttgtgagattctaagagtgtgtgtgcatcttgacaatattagaggaggctgagcccaagcaggcacattctcttcgacccctccctcattcagtctgctttggagtctactgaa
catcaagcttgctatgagcaggatcttagagctgaggaattggcctcccaatccgaacaggtgttataatcctttcttaataggttgtgctgtggacccaatgtgagggctgtgct
ggtgtaaatggtgacatgttgagctggggggatgctttcggggtggggggactggttccattccatcaaaggccctcttgagagtctatccagggacccattgttttactttaac
agaccagaaaagatgtttgttttccatgtcattacccccaggggataccgaatgtgtgggtagaaatttctctgtagattaaaaatcagatttttacatggattcaacaaaggagc
gtcacttggatttttgttttcatccatgaatgtagctgcttctgtgtaaaatgccattttgctattaaaaatcaattcacgctggaaaaaaaaaaaaa
mouse-miat
(SEQ ID NO: 143)
tttgttttttaaatcaagattatagattttggggaggtgactcaggagttaagagcatttgcaactctccaaggaggcttggattcagttcctagtacccacatggtggct
cacaatcatctataacttgagtttcaggaaatccaatactctcttctgacctctacataaaccaggcatgcacatgatgcacataaatattgtgcaggcaaacaccgtcccccaga
cactcaaaatacataaaataaaaataagtattttttaaaagttggaatttggtatgattagctttatagctttagctatttatattagttttatggcaacttgacactacccagagtcatttt
ggaagagggaacctcagttgagaaaaatcctccaccatattgatctatgagcaagccagtaagcagcactccttcatggcctctgcatcagctcctgcttccaggttcctgccc
tggttgagttcctgccttgactttccctcagtgatagagtgttcttgggagtgtagatgaagtaaaccctcgcctcctaaactgcccttggtcatgatcttttatcatagcaatggaa
cccctacttaagacaattatctttgaagcacatattttgacatttaggcatatcaataaatgattgatggttagtttcttaaaatgccccttgaccacaaagacagtgaattttctccctt
gctccctagcatagattataggtctgagccatcagatctggcttgaggttcaaagcttgtgaccacctgttccaaggacaaatgagctgcctagaactcttccactggcctgcct
gtagccgggaatggtggagttccctgtgactgtgggattctatatgagctactgatgagacataagcaagtaaggaaagttgatcttgggatcaacactcatcaagaataagat
gggtcccccactctctagctgtgcttccagacaggtttgcttcagtgtggcttcagcaaagccttcctagtggtctacagtagagaatgcaagagtccatccctagcctgaaca
ctgcacacagacaccaattatgctaaaatatttaatttcattccataaataattaagcctaaataataatacctagatataacttggaacttgtgacaatctacctgcctctgtctctgt
gtaccaggattataggcctgggcacacattcttcttcaggactgattataatacattttgtatattgatttaaaaaaatgatgcttaggtagttatggcctgtgagtaattctataaagt
agactaagtggatatcatcagtctatactctaagacattcctttaatttttttaaatttttttttaaagttctactttttaaagattcctagagagccagatgggtgatgaaaagccaggtt
tcacatgtaacactaatattgggggaaaaaaaacctgacgctagaaattatttagttattctgaaagctgtcacttcttgaaagcagagtcagaattagaaaagaattaacagtgt
atcgttgtgttctcacatttccaatgtggctggtgaatcatgaagcaaaaatttcctcaaaactggagccccagagtcttagtctgagcccatcctcctgcctgagaatctccaaat
tgcctagtgaaggaaaagctatgtccatggtcccacctgttccaaagtccctttggaaatctaggcagaagtaggtttgctagagtcttacttatgacaacctgggaaacaaag
aaggctcttcatacaagggtgtgtatccacacaagaggcagcatcctgaaggaaaccttcaggagcctccgatcctgcagttagctgactgtttcctgccctccttggtccaac
tcgttatggccccaggtgttccttcttgctgctgaactgctgtctctaccagctgagcccactcctgtggtctgcactcactgccctggctgcaggttgaacagggtcaggctact
tctcagatgcttcccaccagccgcagtgaactccattttgacatgaagccatttaaatggaaatccagttttacaaagctcaagtgactggtaatgaagatgtgaatgatgcttat
gaggtttttatttaattttaaatcaattgtgtattctaattcattaataaaggggaaaggaaagaatacaggaaaaaaaaaaaaaaaaa
human-NBAT-1
(SEQ ID NO: 144)
ctagaatgagaatgcacagcaagcggcagacaatcacccggcattcggcatctctttcttttcacgcgctccctcgctccgcctttctccagctctgccttctcaggc
agatacatcagataccttgtttatccatcttcagctccactctgagggcgcagacgcacgattccgggatcgggtgcaccacggcgaagcccaggcgggagacggcagga
gcagctcagatgaagaaactgaaacccacagagatgaagtaacatcctcaagatcaaaaggtaacaaacgattgaggcaggatttggatattgctctacatgacgggaaag
cctgtgctcttggaattacagtcatagtacaacagatcaagttcaaagcatcatctaagtcaaggaaaaccagtaaagaggtttggagagacaaacagggtcaactccaggc
atttgggtcagcagaaacggcacgattgagcagcactgtgactataggatcatggatcagaggctgcttcctctttggttctgggcatcagcctcatgtccactcaaagtaagt
ggcccctctgattggaatcggaggtgcctgggtcatctcacagagccaaacaaatacaattagctattgcaaagccttttgggaattattcccagtgtaaataaacacataacca
tatagcaagagccttgataaagtccaaaaacatgcaaacttggagtatctaagagaaaagaccacaatgtaaatgaaaaaccaaataaactcgggcaaaccataggatagg
gccctgtctgtgatggcctgcatatgatgagccatagaaaaaggatggtgaattctggataataagaaatgtcaatgagatgcaagaaccacctgttttatgtaaagctccaaat
aaccagatcacagtggacagccactcaaataatgccttcataatacagagtattattgagaataactcagttcacagagagcttaaggcagccaatatttgatagcctgtcagaa
aaaaacagaacagtaattatagaaaagaatcatatcctcggaaaaacaaaaattaatcaaactaagtttgtaaagtctatcttacagacacattgtctggactggtcctctcaaaa
atactgtttttttttaatgccaatttgtttagttaatgatttttgtcttattacttcaaaactggaaatatcctatgactcataatatcttacaacctttctactttcttaaagaatctcaagtttat
aatcacaggggatcggattatttttcaaaaattaaatggtgatgtaatgatttctgtgtctattgtagaaaagtcaaccttattacagctgcaacaatggcattaagaaatatgagta
attccaatcaacttgagataatgtctaatcaaacacaaatacaactggtaaatttcattaaatagcatggagattaaattaaaacactattatgtaataaaaacctttagtggtactaa
aattttagaatagttcagatatacagaaaaatttcaaagatacacagagttcccatttttttcctattactaacctctcatatttgtcacaactaatgaatattcaatagagtattattaac
taaagcctatacatttatttagatttccttagtttttagctaacattcttttttttgttccaggatcccatccgggccaccacattgaatttatttgtcattttaggtacctcttggctgtga
gtttcttagactttccttgtttttggtgaccctgacagtttgagggagtactagtcagtcagttatttttgcagaatgccctaaatttgagtttggctgatgtttttcttagggtttgactgg
ggttatgggttttggggaggaagaccacagaggtgaagtaccattctcaccaaattatattaaaggtacataccatcagcatgccttatactattgatgtgaactttgattgcctgg
ctgtggtagtgtttgtcatgtttcttcactgtaaagttactcttctcatcacccacttttctgtactgtactctttggaagaaagtcactatatgcatcccaaatttaaggagtgggaagt
tatgctccacccatttgtaagcagaaaatctacataatttgtttggcattcttctgcataggaaaatcatctcactctcccagttatttatttatttgatctttttttatatcagtatggactc
atgggtatttcttttatactttgggttataatccaatactaacacaataaagaaatttttaatggagatgcaaaaaaaaaaaaaa
human-Malat1
(SEQ ID NO: 154)
cgcagcctgcagcccgagacttctgtaaaggactggggccccgcaactggcctctcctgccctcttaagcgcagcgccattttagcaacgcagaagcccggcg
ccgggaagcctcagctcgcctgaaggcaggtcccctctgacgcctccgggagcccaggtttcccagagtccttgggacgcagcgacgagttgtgctgctatcttagctgtc
cttataggctggccattccaggtggtggtatttagataaaaccactcaaactctgcagtttggtcttggggtttggaggaaagcttttatttttcttcctgctccggttcagaaggtct
gaagctcatacctaaccaggcataacacagaatctgcaaaacaaaaacccctaaaaaagcagacccagagcagtgtaaacacttctgggtgtgtccctgactggctgccca
aggtctctgtgtcttcggagacaaagccattcgcttagttggtctactttaaaaggccacttgaactcgctttccatggcgatttgccttgtgagcactttcaggagagcctggaa
gctgaaaaacggtagaaaaatttccgtgcgggccgtggggggctggcggcaactggggggccgcagatcagagtgggccactggcagccaacggcccccggggctc
aggcggggagcagctctgtggtgtgggattgaggcgttttccaagagtgggttttcacgtttctaagatttcccaagcagacagcccgtgctgctccgatttctcgaacaaaaa
agcaaaacgtgtggctgtcttgggagcaagtcgcaggactgcaagcagttgggggagaaagtccgccattttgccacttctcaaccgtccctgcaaggctggggctcagtt
gcgtaatggaaagtaaagccctgaactatcacactttaatcttccttcaaaaggtggtaaactatacctactgtccctcaagagaacacaagaagtgctttaagaggtattttaaa
agttccgggggttttgtgaggtgtttgatgacccgtttaaaatatgatttccatgtttcttttgtctaaagtttgcagctcaaatctttccacacgctagtaatttaagtatttctgcatgt
gtagtttgcattcaagttccataagctgttaagaaaaatctagaaaagtaaaactagaacctatttttaaccgaagaactactttttgcctccctcacaaaggcggcggaaggtga
tcgaattccggtgatgcgagttgttctccgtctataaatacgcctcgcccgagctgtgcggtaggcattgaggcagccagcgcaggggcttctgctgagggggcaggcgga
gcttgaggaaaccgcagataagtttttttctctttgaaagatagagattaatacaactacttaaaaaatatagtcaataggttactaagatattgcttagcgttaagtttttaacgtaatt
ttaatagcttaagattttaagagaaaatatgaagacttagaagagtagcatgaggaaggaaaagataaaaggtttctaaaacatgacggaggttgagatgaagcttcttcatgg
agtaaaaaatgtatttaaaagaaaattgagagaaaggactacagagccccgaattaataccaatagaagggcaatgcttttagattaaaatgaaggtgacttaaacagcttaaa
gtttagtttaaaagttgtaggtgattaaaataatttgaaggcgatcttttaaaaagagattaaaccgaaggtgattaaaagaccttgaaatccatgacgcagggagaattgcgtca
tttaaagcctagttaacgcatttactaaacgcagacgaaaatggaaagattaattgggagtggtaggatgaaacaatttggagaagatagaagtttgaagtggaaaactggaa
gacagaagtacgggaaggcgaagaaaagaatagagaagatagggaaattagaagataaaaacatacttttagaagaaaaaagataaatttaaacctgaaaagtaggaagc
agaagaaaaaagacaagctaggaaacaaaaagctaagggcaaaatgtacaaacttagaagaaaattggaagatagaaacaagatagaaaatgaaaatattgtcaagagttt
cagatagaaaatgaaaaacaagctaagacaagtattggagaagtatagaagatagaaaaatataaagccaaaaattggataaaatagcactgaaaaaatgaggaaattattg
gtaaccaatttattttaaaagcccatcaatttaatttctggtggtgcagaagttagaaggtaaagcttgagaagatgagggtgtttacgtagaccagaaccaatttagaagaatac
ttgaagctagaaggggaagttggttaaaaatcacatcaaaaagctactaaaaggactggtgtaatttaaaaaaaactaaggcagaaggcttttggaagagttagaagaatttg
gaaggccttaaatatagtagcttagtttgaaaaatgtgaaggactttcgtaacggaagtaattcaagatcaagagtaattaccaacttaatgtttttgcattggactttgagttaaga
ttattttttaaatcctgaggactagcattaattgacagctgacccaggtgctacacagaagtggattcagtgaatctaggaagacagcagcagacaggattccaggaaccagtg
tttgatgaagctaggactgaggagcaagcgagcaagcagcagttcgtggtgaagataggaaaagagtccaggagccagtgcgatttggtgaaggaagctaggaagaagg
aaggagcgctaacgatttggtggtgaagctaggaaaaaggattccaggaaggagcgagtgcaatttggtgatgaaggtagcaggcggcttggcttggcaaccacacgga
ggaggcgagcaggcgttgtgcgtagaggatcctagaccagcatgccagtgtgccaaggccacagggaaagcgagtggttggtaaaaatccgtgaggtcggcaatatgtt
gtttttctggaacttacttatggtaaccttttatttattttctaatataatgggggagtttcgtactgaggtgtaaagggatttatatggggacgtaggccgatttccgggtgttgtaggt
ttctctttttcaggcttatactcatgaatcttgtctgaagcttttgagggcagactgccaagtcctggagaaatagtagatggcaagtttgtgggttttttttttttacacgaatttgagg
aaaaccaaatgaatttgatagccaaattgagacaatttcagcaaatctgtaagcagtttgtatgtttagttggggtaatgaagtatttcagttttgtgaatagatgacctgtttttactt
cctcaccctgaattcgttttgtaaatgtagagtttggatgtgtaactgaggcgggggggagttttcagtatttttttttgtgggggtgggggcaaaatatgttttcagttctttttccctt
aggtctgtctagaatcctaaaggcaaatgactcaaggtgtaacagaaaacaagaaaatccaatatcaggataatcagaccaccacaggtttacagtttatagaaactagagca
gttctcacgttgaggtctgtggaagagatgtccattggagaaatggctggtagttactcttttttccccccacccccttaatcagactttaaaagtgcttaaccccttaaacttgttat
tttttacttgaagcattttgggatggtcttaacagggaagagagaggggggggagaaaatgtttttttctaagattttccacagatgctatagtactattgacaaactgggttaga
gaaggagtgtaccgctgtgctgttggcacgaacaccttcagggactggagctgcttttatccttggaagagtattcccagttgaagctgaaaagtacagcacagtgcagctttg
gttcatattcagtcatctcaggagaacttcagaagagcttgagtaggccaaatgttgaagttaagttttccaataatgtgacttcttaaaagttttattaaaggggaggggcaaata
ttggcaattagttggcagtggcctgttacggttgggattggtggggtgggtttaggtaattgtttagtttatgattgcagataaactcatgccagagaacttaaagtcttagaatgg
aaaaagtaaagaaatatcaacttccaagttggcaagtaactcccaatgatttagtttttttccccccagtttgaattgggaagctgggggaagttaaatatgagccactgggtgta
ccagtgcattaatttgggcaaggaaagtgtcataatttgatactgtatctgttttccttcaaagtatagagcttttggggaaggaaagtattgaactgggggttggtctggcctact
gggctgacattaactacaattatgggaaatgcaaaagttgtttggatatggtagtgtgtggttctcttttggaatttttttcaggtgatttaataataatttaaaactactatagaaactg
cagagcaaaggaagtggcttaatgatcctgaagggatttcttctgatggtagcttttgtattatcaagtaagattctattttcagttgtgtgtaagcaagtttttttttagtgtaggaga
aatacttttccattgtttaactgcaaaacaagatgttaaggtatgcttcaaaaattttgtaaattgtttattttaaacttatctgtttgtaaattgtaactgattaagaattgtgatagttcag
cttgaatgtctcttagaggggggcttttgttgatgagggaggggaaactttttttttttctatagacttttttcagataacatcttctgagtcataaccagcctggcagtatgatggcc
tagatgcagagaaaacagctccttggtgaattgataagtaaaggcagaaaagattatatgtcatacctccattggggaataagcataaccctgagattcttactactgatgagaa
cattatctgcatatgccaaaaaattttaagcaaatgaaagctaccaatttaaagttacggaatctaccattttaaagttaattgcttgtcaagctataaccacaaaaataatgaattga
tgagaaatacaatgaagaggcaatgtccatctcaaaatactgcttttacaaaagcagaataaaagcgaaaagaaatgaaaatgttacactacattaatcctggaataaaagaag
ccgaaataaatgagagatgagttgggatcaagtggattgaggaggctgtgctgtgtgccaatgtttcgtttgcctcagacaggtatctcttcgttatcagaagagttgcttcatttc
atctgggagcagaaaacagcaggcagctgttaacagataagtttaacttgcatctgcagtattgcatgttagggataagtgcttatttttaagagctgtggagttcttaaatatcaa
ccatggcactttctcctgaccccttccctaggggatttcaggattgagaaatttttccatcgagcctttttaaaattgtaggacttgttcctgtgggcttcagtgatgggatagtacac
ttcactcagaggcatttgcatctttaaataatttcttaaaagcctctaaagtgatcagtgccttgatgccaactaaggaaatttgtttagcattgaatctctgaaggctctatgaaagg
aatagcatgatgtgctgttagaatcagatgttactgctaaaatttacatgttgtgatgtaaattgtgtagaaaaccattaaatcattcaaaataataaactatttttattagagaatgtat
acttttagaaagctgtctccttatttaaataaaatagtgtttgtctgtagttcagtgttggggcaatcttgggggggattcttctctaatctttcagaaactttgtctgcgaacactcttta
atggaccagatcaggatttgagcggaagaacgaatgtaactttaaggcaggaaagacaaattttattcttcataaagtgatgagcatataataattccaggcacatggcaatag
aggccctctaaataaggaataaataacctcttagacaggtgggagattatgatcagagtaaaaggtaattacacattttatttccagaaagtcaggggtctataaattgacagtg
attagagtaatactttttcacatttccaaagtttgcatgttaactttaaatgcttacaatcttagagtggtaggcaatgttttacactattgaccttatatagggaagggagggggtgc
ctgtggggttttaaagaattttcctttgcagaggcatttcatccttcatgaagccattcaggattttgaattgcatatgagtgcttggctcttccttctgttctagtgagtgtatgagacc
ttgcagtgagtttatcagcatactcaaaatttttttcctggaatttggagggatgggaggagggggggggcttacttgttgtagctttttttttttttacagacttcacagagaatgca
gttgtcttgacttcaggtctgtctgttctgttggcaagtaaatgcagtactgttctgatcccgctgctattagaatgcattgtgaaacgactggagtatgattaaaagttgtgttcccc
aatgcttggagtagtgattgttgaaggaaaaaatccagctgagtgataaaggctgagtgttgaggaaatttctgcagttttaagcagtcgtatttgtgattgaagctgagtacattt
tgctggtgtatttttaggtaaaatgctttttgttcatttctggtggtgggaggggactgaagcctttagtcttttccagatgcaaccttaaaatcagtgacaagaaacattccaaacaa
gcaacagtcttcaagaaattaaactggcaagtggaaatgtttaaacagttcagtgatctttagtgcattgtttatgtgtgggtttctctctcccctcccttggtcttaattcttacatgca
ggaacactcagcagacacacgtatgcgaagggccagagaagccagacccagtaagaaaaaatagcctatttactttaaataaaccaaacattccattttaaatgtggggattg
ggaaccactagttctttcagatggtattcttcagactatagaaggagcttccagttgaattcaccagtggacaaaatgaggaaaacaggtgaacaagctttttctgtatttacatac
aaagtcagatcagttatgggacaatagtattgaatagatttcagctttatgctggagtaactggcatgtgagcaaactgtgttggcgtgggggtggaggggtgaggtgggcgc
taagcctttttttaagatttttcaggtacccctcactaaaggcaccgaaggcttaaagtaggacaaccatggagccttcctgtggcaggagagacaacaaagcgctattatccta
aggtcaagagaagtgtcagcctcacctgatttttattagtaatgaggacttgcctcaactccctctttctggagtgaagcatccgaaggaatgcttgaagtacccctgggcttctc
ttaacatttaagcaagctgtttttatagcagctcttaataataaagcccaaatctcaagcggtgcttgaaggggagggaaagggggaaagcgggcaaccacttttccctagcttt
tccagaagcctgttaaaagcaaggtctccccacaagcaacttctctgccacatcgccaccccgtgccttttgatctagcacagacccttcacccctcacctcgatgcagccagt
agcttggatccttgtgggcatgatccataatcggtttcaaggtaacgatggtgtcgaggtctttggtgggttgaactatgttagaaaaggccattaatttgcctgcaaattgttaac
agaagggtattaaaaccacagctaagtagctctattataatacttatccagtgactaaaaccaacttaaaccagtaagtggagaaataacatgttcaagaactgtaatgctgggt
gggaacatgtaacttgtagactggagaagataggcatttgagtggctgagagggcttttgggtgggaatgcaaaaattctctgctaagactttttcaggtgaacataacagactt
ggccaagctagcatcttagcggaagctgatctccaatgctcttcagtagggtcatgaaggtttttcttttcctgagaaaacaacacgtattgttttctcaggttttgctttttggcctttt
tctagcttaaaaaaaaaaaaagcaaaagatgctggtggttggcactcctggtttccaggacggggttcaaatccctgcggcgtctttgctttgactactaatctgtcttcaggact
ctttctgtatttctccttttctctgcaggtgctagttcttggagttttggggaggtgggaggtaacagcacaatatctttgaactatatacatccttgatgtataatttgtcaggagcttg
acttgattgtatattcatatttacacgagaacctaatataactgccttgtctttttcaggtaatagcctgcagctggtgttttgagaagccctactgctgaaaacttaacaattttgtgta
ataaaaatggagaagctctaaattgttgtggttcttttgtgaataaaaaaatcttgattggggaaaaaa
mouse-Malat1
(SEQ ID NO: 146)
caggcattcaggcagcgagagcagagcagcgtagagcagcacagctgagctcgtgaggcaggagactcagcccgaggaaatcgcagataagtttttaattaaa
aagattgagcagtaaaaagaattagaactctaaacttaagctaatagagtagcttatcgaaatattacttagtcttaataatctaagaagatcttaagagataacatgaaggcttatt
taaacagtttgaaaaaggaaatgaggagaaaagtatttgtactgtataatggaggctgaccagagcagtttaggagattgtaaagggaggttttgtgaagttctaaaaggttcta
gtttgaaggtcggccttgtagattaaaacgaaggttacctaaatagaatctaagtggcatttaaaacagtaaagttgtagagaatagtttgaaaatgaggtgtagttttaaaagatt
gagaaaagtaggttaagttgacggccgttataaaaatccttcgactggcgcatgtacgtttgaaggcatgagttggaaacagggaagatggaagtgttaggctagccgggcg
atggtggcgcacgcctttaatcctagcacttgggaggcagaggcaggcggatttctgagttcgaggccagcctggtctacagagtgagttccaggacagccagggctacac
agagaaaccctgtcttgaaaaaacaaaaaggttaggctagtatttggagaaagaagattagaaaatggaagtgaaagacgaagaagacatacaggaaggtgaagaaaaag
ctgttagagaagataggaaaatagaagacaaagcatctttagaagacagaaaaggtacttaaaggcacaggtagtaggaagccgaagaatagaagatagaaagaagcaa
gatagaaaaacaaaatggaagttaagacaactttggatgccagcattcaagataggcaaagaagataagattgaggccaaaaggttggataagatataaagtcagaaggaa
attatctttaaagccataagttcaaatttctgatggagcgagcagtttagaagagtctttagacagccacatacaagattgaagctagcaatcaaagctactaggactgaagtaa
aaagttaaggcagaatgcctttgaagagttagaagaatattaaaagccttaacttgtagcttaattttgcttgatgacaaaaggacttttgataacagtttcaagattgtcagcatttt
gcattggacttgagctgaggtgcttttaaaatcctaacgactagcattggcagctgacccaggtctacacagaagtgcattcagtgaactaggaagacaggagcggcagaca
ggagtcccgaagccagtttggtgaagctaggaaggactgaggagccagcagcagcagtgcatggtgaagatagcccaggaaagagtgcggttcggtggaggaagctag
gaagaaggagccatacggatgtggtggtgaagctgggaaagggttccaggatggtggagcgagagcgagttggtgatgaagctagctggcggcttggcttgtcaactgc
gcggaggaggcgagcaggcattgtggagaggatagatagcggctcctagaccagcatgccagtgtgcaagaaaggctgcagggagagcatgcggtgcggtaacattcc
ttgaggtcggcaacatggtggtggttttctgtaacttggatggtaacttgtttactttgtcttaatagttatgggggagttgtaggcttctgtgtaaagagatatatctggggctgtat
gtaggcctttgcgggtgttgtaggtttttctttttcagggttatgtcctcttgcatcttgtcagaagcttttgagggctgactgccaaggcccagaaagaagaatggtagatggcaa
gttgtctttaaccgctcagaggggaatgaatggtagagccagcacaacctcccagttttgtaagacgttgtagtttgaacagatgacctaccacaagcctcactcctgtgtagg
ggaggtaattgggcaaagtgcttttgggggaatgggggcaaaatatattttgagttcttttccccttaggtctgtctagaatcctaaaggcagatgactcaagggaaccagaaa
aaaggaaatccactctcaggataagcagagctcgccaggtttacagtttgtaggaagtagaggatggatgctagctttcacactgagtgtggaggagctggccatggcgga
attgctggtagtttactctttccccctcccttaatgagatttgtaaaatcctaaacacttttacttgaaatatttgggagtggtcttaacagggaggagtggggggggaaacgttttt
tttctaagattttccacagatgctatagttgtgttgacacactgggttagagaaggcgtgtactgctatgctgttggcacgacaccttcagggactggagctgccttttgtccttgg
aagagttttcccagttgccgctgaagtcagcacagtgcggctttggttcacagtcacctcaggagaacctcaggagcttggctaggccagaggttgaagttaagttttacagc
accgtgatttaaaatatttcattaaaggggaggggtaaaacttagttggctgtggccttgtgtttgggtggggggggtgttaggtaattgtttagtttatgatttcagataatcatac
cagagaacttaaatatttggaaaaacaggaaatctcagctttcaagttggcaagtaactcccaatccagtttttgcttcttttttcctttttctttttttgaggcgggcagctaaggaag
gttggttcctctgccggtccctcgaaagcgtagggcttgggggttggtctggtccactgggatgatgtgatgctacagtggggactcttctgaagctgttggatgaatatagatt
gtagtgtgtggttctcttttgaaatttttttcaggtgacttaatgtatcttaataactactataggaacaaaggaagtggctttaatgaccctgaaggaatttcttctggtgatagctttta
tattatcaagtaagagatactatctcagttttgtataagcaagtctttttcctagtgtaggagaaatgattttccttgtgactaaacaagatgtaaaggtatgctttttttcttcttgtgcat
tgtatacttgtgtttatttgtaacttataatttaagaattatgataattcagcctgaatgtcttttagaggggggcttttgttgatgagggaggggaaacctttttttttctgtagacctttt
tcagataacaccatctgagtcataaccagcctggcagtgtgatgacgtagatgcagagggagcagctccttggtgaatgagtgataagtaaaggcagaaaaaataatgtcat
gtctccatggggaatgagcatgagccagagattgttcctactgatgaaaagctgcatatgcaaaaatttaagcaaatgaaagcaaccagtataaagttatggcaatacctttaa
aagttatggcttatctaccaagctttatccacaaaagtaaagaattgatgaaaaacagtgaagatcaaatgttcatctcaaaactgcttttacaaaagcagaatagaaatgaagtg
aaaatgctgcattaagcctggagtaaaaagaagctgagcttgttgagatgagtgggatcgagcggctgcgaggcggtgcagtgtgccaatgtttcgtttgcctcagacaggtt
tctcttcataagcagaagagttgcttcattccatctcggagcaggaaacagcagactgctgttgacagataagtgtaacttggatctgcagtattgcatgttagggatagataagt
gccttttttctctttttccaaaaagacctgtagagctgttgaatgtttgcagctggcccctcttaggcagttcagaattttgagtagttttcccatccagcctcttaaaaattcctaagc
cttgcaccgatgggctttcatgatgggatagctaataggcttttgcatcgtaaacttcaacacaaaagcctacatgattaatgcctactttaattacattgcttacaagattaaggaa
tctttatcttgaagaccccatgaaagggatcattatgtgctgaaaattagatgttcatattgctaaaatttaaatgtgctccaatgtacttgtgcttaaaatcattaaattatacaaatta
ataaaatacttcactagagaatgtatgtatttagaaggctgtctccttatttaaataaagtcttgtttgttgtctgtagttagtgtgggcaattttggggggatgttcttctctaatcttttc
agaaacttgacttcgaacacttaagtggaccagatcaggatttgagccagaagaccgaaattaactttaaggcaggaaagacaaattttattctccatgcagtgatgagcattta
ataattgcaggcctggcatagaggccgtctaactaaggactaagtaccttaggcaggtgggagatgatggtcagagtaaaaggtaactacatattttgtttccagaaagtcag
gggtctaatttgaccatggctaaacatctagggtaagacacttttcccccacatttccaaatatgcatgttgagtttaaatgcttacgatcatctcatccactttagccttttgtcacct
cacttgagccacgagtggggtcaggcatgtgggtttaaagagttttcctttgcagagcctcatttcatccttcatggagctgctcaggactttgcatataagcgcttgcctctgtct
tctgttctgctagtgagtgtgtgatgtgagaccttgcagtgagtttgtttttcctggaatgtggagggagggggggatggggcttacttgttctagctttttttttacagaccacaca
gaatgcaggtgtcttgacttcaggtcatgtctgttctttggcaagtaatatgtgcagtactgttccaatctgctgctattagaatgcattgtgacgcgactggagtatgattaaagaa
agttgtgtttccccaagtgtttggagtagtggttgttggaggaaaagccatgagtaacaggctgagtgttgaggaaatggctctctgcagctttaagtaacccgtgtttgtgattg
gagccgagtccctttgctgtgctgccttaggtaaatgtttttgttcatttctggtgaggggggttgggagcactgaagcctttagtctcttccagattcaacttaaaatctgacaaga
aataaatcagacaagcaacattcttgaagaaattttaactggcaagtggaaatgttttgaacagttccgtggtctttagtgcattatctttgtgtaggtgttctctctcccctcccttgg
tcttaattcttacatgcaggaacattgacaacagcagacatctatctattcaaggggccagagaatccagacccagtaaggaaaaatagcccatttactttaaatcgataagtga
agcagacatgccattttcagtgtggggattgggaagccctagttctttcagatgtacttcagactgtagaaggagcttccagttgaattgaaattcaccagtggacaaaatgagg
acaacaggtgaacgagccttttcttgtttaagattagctactggtaatctagtgttgaatcctctccagcttcatgctggagcagctagcatgtgatgtaatgttggccttggggtg
gaggggtgaggtgggcgctaagcctttttttaagatttttcaggtacccctcactaaaggcactgaaggcttaatgtaggacagcggagccttcctgtgtggcaagaatcaagc
aagcagtattgtatcgagaccaaagtggtatcatggtcggttttgattagcagtggggactaccctaccgtaacaccttgttggaattgaagcatccaaagaaaatacttgagag
gccctgggcttgttttaacatctggaaaaaaggctgtttttatagcagcggttaccagcccaaacctcaagttgtgcttgcaggggagggaaaagggggaaagcgggcaacc
agtttccccagcttttccagaatcctgttacaaggtctccccacaagtgatttctctgccacatcgccaccatgggcctttggcctaatcacagacccttcacccctcaccttgatg
cagccagtagctggatccttgaggtcacgttgcatatcggtttcaaggtaaccatggtgccaaggtcctgtgggttgcaccagaaaaggccatcaattttccccttgcctgtaat
ttaacattaaaaccatagctaagatgttttatacatagcacctatgcagagtaaacaaaccagtatgggtatagtatgtttgataccagtgctggggggaatgtaggaagtcgg
atgaaaagcaagcctttgtaggaagttgttggggtgggattgcaaaaattctctgctaagactttttcaggtggacataacagacttggccaagctagcatcttagtggaagcag
attcgtcagtagggttgtaaaggtttttcttttcctgagaaaacaaccttttgttttctcaggttttgctttttggcctttccctagctttaaaaaaaaaaaagcaaaagacgctggtgg
ctggcactcctggtttccaggacggggttcaagtccctgcggtgtctttgcttgactcttatatcatgaggccattacatttttcttggagggttctaaaggctctgggtatggtagc
tgatatcactggaacactccccagcctcagtgttgaactcttgataattaactgcattgtctttcaggttatgcccaattcgtcttattacctctgagtcgacacacctcctactatttat
tgaatactttgattttatgaaataaaaactaaatatctctca
mouse-Dlx1as
(SEQ ID NO: 147)
agctcagagatgcaaaaagcctgcggcgctctggaggcgcctggtcctggggacctctttcctccactctgttctcagcataggaagatggggctggggaattctt
tttggttttctggttttgttttgctctggttttggtcggggttttgtatttttctattcttccttggttttattttttcctttttttcctttttttttttcttttgctttcctgtcattctcccagccaaaagccgccttcg
acccttttgatttgatgtagcaaagagtgtgtgtccagattatctgaaatagacactgaagaagctacatagatggtcaagcagggcccggaagaagaccattca
ggaaaaagtggtccaggactcggtggatgggctgccggcagaccggtgtggctcagacctggtgactttttggagaggtcgtagaatgaggacagcagacacacgagga
ccttgacttcggcgcacagacagtgcaaaaaggagacaaagacggacagaatcaaaaaagagcgtatttccccctcctacgtatttgtttttcctgatagtctcacaaagatga
gagtctggtccggtccagcgggttatcttgtggcactggctgtctccagtggctgtgcctgcgctttgaggccactctgctgttcaccgtggcccaaaccagattcgggctgaa
aggtcgctgagtcagacttttaggggctccgagaagcagcaagccctctgccttcactgttcctttttcttttatttttagctccccacccccacccctgctcccgctcgtgggctc
ttctcctcctggcccagggcgtgcgaaaaggtggatgaacctggagggggaggggccgggccaagggagacgggcaggaagcgggtgagtgcgaactgataacctc
acatcagttgaggctgctgcatagcttcttggtgcgctgagggataccatgatgtatagctggggacgtaggagccagcgctgctgccggagcccttcccagatgaggagtt
cggattccagccgggtggtaccggtggggagccggcagacaaggctctgccgttcgccagcgcgctgccctccagagctgccccgccttgcttcatcagcttcttgaactt
ggagcgtttgttctggaaccatatcttgacctgcaggatcgggcgacaacgtgactggactatgaccattgagcctacccaggtccaaggagaaaagggacgggtggaca
acaatacccgccctccttgctttgggaaagctgacagctccaggacagaggggcagagaccaccgcctccccacaccccatgagggccaaaggccagaaaattgagccc
caggtccagccgctgttggcaacttttagagcagctatcagagccgggaataagagaacaggggagagccaggccttggcttgcaaatgagacgatgccacctgggcga
cgtttatggaagacctcatgcagcacaatgcaataacaataaagaaaatttaaaacctagataggaacgggggggggggggggcatttgttaaaattagatctcctttcctc
gtaggactattggaagaggctacagcgtctttgttaaactctttagactcaaaccaattctcccccccacccccaacggtgtatttgctttccccagctccaacctaaatctagag
acagactggccctcgttcgaatgcctaggaactgcccagggtcttagagaagtgcaggaagaaaaaagaaaaatctcactttttttttcccccttcttcttaaggatctggaactt
gggaagtgaagggccagagggaaggatctgcctctgccacctcgcaagaagaaggaggtgggggaagcaggcttaggcccaggacctgagtgattagtggcacacttt
aaaggctaaggaagattaaatcccccttctattgttccagttgttcaggcacgtgtgacctcagaacctccaaacaaaacgtctggggaaatccttccagttccctgcctggtca
gaagaccaggtagcctgcaggtcagagagtagggtccgagctagggcaaacagtttgagggaggaagaggagacgctgaaagtcagaggtggatacacacacacaca
cacacacacacacacacacacgggttggggggggactgtgacacctagaaccagaggcccagcgaacaaaaaatgtgaagcctggggctggagcccagctcccgag
gcttgtttggcagagcggccaggtacctgcgtctgtgtgagtcccaaggaggccgccagctccgccctctcaggcagagctaagtactgagtttgttggaacctccggttca
aagcctgcaactgcaaactggaataaattgtcctgggtttacggatctttttccctttgccgttaaagcgcacctcgccgccttccaccaccgtactcttctcggagtccgcccct
ggagggacaacagaagcagagacccttgttaatgctgcactgcctttagtgaggagggacgagaatgaatgtctatggatgggttggcagaagtagggtccggaaaagaa
cgttcaagaaaccctgaaagactgcgaaggtccctcaaggtccgagcgcacgtccctggctgctgcagacagaattgggtcgttcccaagagcgcagctattgcctttgca
gagaaagtttgctattttttgcaggcggtagttgaggtttaattacccttaattgcatacattgtttatagcgctacgcaataaataattaccgag
human-Six3os
(SEQ ID NO: 148)
ttttttttatctttgcaactcttaatctcgctacctccccctcctctcttctctcttctccctctctctcctcttttctctaccgctgtctctgagacttcttcttccctttttcctaaag
aagttgatccagaaattcgaaaagccctgggcagagttgttgaatgggatgtgcaattagagaggggattttgttgggaggcaagagggtcccccaggctccacatagtcca
ggtcggcaggcaaggaaagacggctgaccagcccagcgggcgcggtttgcacatggtttgcacgtcgggccgcctctttctgcctataattcagctgcctctctgaggagt
ggtggaggagctgcgggagccgagaagcccaagagccctctggacccagaaaagtcctacagatgcccactcctcaccacacaacagaaggagctctggtcctgcctg
ccagccccagagggcactcaaactttggaggccagccacccggatgaagtgaataaggcctgaaaattgcttgtttgctcgtattgtaaaataataataattaccattattattttt
aaactgtctaatttctctagggaaagtaacatcgaaagcctaaaacagacgccaaaaggcccatagaacacagagggccctctctgcctctggccaccacagcccctaggc
caggcatgggtatttattcttaggtatgttgcttttaagaagctgtaatcagcatcttgagccgggcctccctttgtgaggcttctgtaactatggaagtgtgatttacgcagatttgt
cggggtcagagacgtctttccctgagcactgtgtatatttagacaggactcggtttggtgttaaaaagtgtatatgttgaatggattcacacacagtagccaacaatgaccacatt
gtcggcccgtgtacaacgcgtattgaaacgcagcgcccagacttcaactaatctgccctcaataaagctgaaataattatcctaa
mouse-Six3os
(SEQ ID NO: 149)
aaaaccctcgccactcatgaagaactgaggcaaaaggatcacagatgctgctcggggccagccctatactccctccagtgcctggtctccggtggacccgcgg
ggctctgcagttctctggggccgcgccttgtaagcgctaagccgggaggagggcgcaggcagggcgcacagctctgagcgcgaccctacccggctcgggcccgcag
caaccccgactgtccgagagcctctccccaagcaaggctctgcgccgccctctgagcccacctcctggccacaacgctgcctgcgcttctccagcccaggccggaagccc
tatgctgcctcccgaagccggactttctggaagcccatcttcgtatgctcaccagcccaccctcctacgctggtcagagtgagattgctaacctcaagaccccattcaatgcca
gctttcaactactgctgaagaccaatgtaggtcctggaatagaagaactgcagagtatccggagctggccgaccccagactgattctcaacaggtggctgggtagtgccccc
caccctgtgttggtcgtgcgggacccatcgcacacagagaagccaccttacccctcacctcaccccgtcggccaaagactgaccacccgcgcccgagtgtggggagggg
tgggaattccggatcacacacacacacacacacacacacacacaccagggtaggggaacagggaagatcttacctgcagaaaggtaagagaagccgggagcgtgtagc
agcggcggataaatattcattaggaagtgtaagtcctttcaaagggctccgcgcgcgcgctctagctcctgtctctctttctctccctccctctgtcctccaggaaggcttcccct
cctctagctatctcaacgcccccccccccttcctgcgaaataccgggtggatcctaggctagctaacctgttgggaagcctgaactgcggtagcgcctctgcagctagcagtt
tgctttcgctcctggggcgcgcgtccccccccatcccctcccctccttcctttcgcccagcccctcacccctcctaaatcactacatcattagctttattggggggggggggg
cgggtaacggagcagccagacgcacctctccatagaagggtgaatagttggtgccctgcgatgcaaccccaactccatctccacctcattctcacccatctgcacatcctcat
ccaatcttgcttctttttcttcgctctttccacctccacctccatcctgtgtcaacctaactccctcagcagccctcccacccgccgccgccaatccggatcacagattgggttggg
agtgagattgcaaggctgttaggtccgaaggtcctgtgtgtcgaggttagaactcctccctggcgtgagtacggggtggttatcaagatctccttctccttcctgaccctgcaag
gttaccaaacagcctggacttttgaatcagatctctggagcaagcattaagaactccagccacacagatctcagggcctggccctttcactcagccgcggaggctgcttgag
ggatggggagaggtgatttcactcagcgccaacgcgcaggaaaaactcatagacgacagtcggactaatactcgtcccttaagaatgcactacctgaagttagtcgcaagtc
atcttcaatcgaggaaaggaaaaaaaaaatcctcacatctagaagcttcgccgaagccacttccaacagcacgcgaacatgttcgcgcacacccttctctgtgcacgcccca
aagcctgctctctcgcgggcaggtgaaaatacaggctgcccaggccagaggagagaaggggcggaggaatacatcgcttagggttgttgagaaactaggaataactgtt
ttcaatactctgctctgctaccaggaccttaggtccctcttgggaccctgaaaagtggctttgccccacgatgggggggggggggcagtgactctagctctggccctcctact
gggagcttaaagttggggtgaagggtttaaggaggagccgaggggggggccctgttagtttgcagttgtaagtgcagctgtgcctgggacaccaagaaggagagtctcg
ccccctaatgctcacggtgcttgcttagaggacccccgaggttaactaccggagcttctgctctctcctgagccccaggccaacacaggacgatggtggccgggatgccag
ccgggatgcctaaacacagtaagacttcagttgcctctcatttggctgttcagagctaatgtctaccagaggaggttttagaagtgggtcgcacccgtcctgaatccatcctgtg
aggtggcagagggtcagagggtcactccttcttgttaaaggttagcataataagtattattattactagggtagaggaagatgtggaggaaaaaaaaagcctagggtgcctag
gacactaaccaaccttacagcttacccaaagtccagaacctcccattgtacaccttctctccccaacacagatccctaaggtctcctcttcatctggcatgtgtgttcacagaagg
tacagtgtgcgagaacacacttaggaaattttaggaacgctaaccacagtactggaggaataaagatgtgtcgaaggaatggatggaatgcatgaacacatacaccttgttgt
ccagatgcagacaaaagctaccatgtccgcttggagtgacgcccagacacgcacagagatgtacaggaagccaaggagagtgggatccaccagagtacagtacaagtac
agagtacagtgcaagatgcccagtggccctggagaaaaaccttcgttgactcttctgtatggaaagtttctgaagaaagggccaaggaatgatctgccccagaggctgccttt
aggctcccatacctgctctgtttggagacacccagaagaggccagtacagcaaagactctttttggtttggttatttctgttgcttgttgtctttttttcatcctttgcctgggaaattgt
gctctggaatagggcttgagtagacagcacaggcctccagtagataacaagagattctcaatctccaatgaggacattccgttctaaggaagcttccagataggggaaagcc
ctgttttgtcccccttttctcctctgagatctgaaaaaatagaaagaaaaaaaaagaaaaacaaaacaaatataaaaactatagcccaatcccatgtacatcaaataaatccacaa
ggaatatgctagatcaagctaaattatgtatttaactcattttcttttgtgagatactgagaagtcttcaacttcctctattccaggcagggatcgaggacctggcctggcagaggtt
ccaccaccagggtttcccacagtggttctgggactaggcgtaaggtgtcctgacttgttttctctgtcacctcttctggggaactggggaactgtcccagacaatggctgcccc
agttccctctagaagagacagcccccacccccagaacccgttgccctccaacctcacctcattttctcttgagctcattctccgagctcttaaagtagtaggtgtgagtcttggac
catttctttaaaagaaacaactaaggaaagggagggaggggaggagggaggaggggagcgggaaggagagctacctcctagactaacgcgtccaggaaccactctcta
aggagcaaaagagagcctgtgttgtaaagtagaagtgatcccccgacattgctgcagggcgccttgcaacagatgtaactgtccactgaagagacagaggggggcatgg
ggaagattttttttcctcccttttgtgaacagctcttacacttttccaagacacattgagttcctggacatttttgtgaatttgttcttgattgtgttttgaacaataaactgtgaaaatcaa
cttcaagcccacacaatcagaatctttcttgggagggcaggttctttcagcgctgtttttctccaggtcgcttttatcttacccccaatgggagtcacctgctgatgctcctcggga
ggagactgtaactgaaagacggaagtgaatgcagacacagcatcctctgagccaaaccgttgctttggttccttctagcttaggatgccgaacactggctggctttgaacacc
ctctacttcgctaagaagtttagagcctttctcacaacaaaccacagcgttttagaactggaaatgtttcttctgattttccagtcttaggacaattttcgaactctaacttaaaaaaaa
atttgtttatgggggtttagctagaacacttgtagcattcgcttctcaaggaaagatcaactgttttctcataaccccaactgctccacacacttcataggatataatgtattttctttac
ttatccttgtgccttctgcccaggacatagaactaggggcaaaactttggaggtctatgacctcttgatagcacctgtaattgttaggcttttgttcttgtgccttgtaaatctctcatg
tacgtctatgaaaatatgcattgcatgggtgtgatcctatcaagttgtccacactttctcaacaaccactgatgagtgaaaccatttctattgcccagggcaaagggctaatcgcc
ttgtgggtctagagttgtcataggataatatccggtgtgtgtgtgatatgacagttattactttcaagaataaattaaaataaccagatacccctttaatactttcctcaacagcgtca
atgctctagagatgggacctcgccttccaaggtgtcttcactatcacagggcccgggctctctgcctcagcccaaccctgggcttgcgctcagagaggaggtattccgacgg
aaaaaaatcccatacatttttaactacagtgattactaaattaatagcaggcaccaagacagatgtcaaaacgtcttgaaaatgtttatctgtgaataattgagaaggtagtggga
cgcattatctgattgtgtaatgaaatatgtatgaggaacagctgtttgcagcagccccttccccttgccgccttagccagggcccttctctcgcaagctttcttctactttcttccac
ctttttcttccttctactttctagttcgctctcttcttccttaactgctcgcacggctttggctgcagtgaacccagagtcgcctggttctctgatgaaggttagccacaaggtttcctct
aagccttgctggattggcgttccgtagtgggatgaggcttcgttgtctgctgttctggccatcgcgccttccggccaggctgcccgctgggtctccatggagtctgcggg
human-Evf2
(SEQ ID NO: 150)
atttcacacctggatgtgctcactcaaccaagaatatagagaaagagcttctgccctgagactcagaaaaatattctcctgtgctttggttcagtatagatttctaaacc
ctgatcattgcttaagagatattcactgaggacagagtcttgctctattgcccaggctgcaactggtgtgatctcggctcactacaacctctgcctcctgggttcaagcgattctc
ctgcctcaagctcccaagtagctgggattacaagcatgcaccaccatgcctggctaatttttgtatttttagtggagacggggtttcgccacattggccagggtggtcttgaactc
ctgacctcaagtgatccacctgccttggcctcccaaagtgctgggattataagcatgagccactgcacccagccttatactgaactttcaatgggttcaattccactaggagcat
aaaggccactgcatatgagttgtggaaagaagagattagaagaaggaagaacttgagatgagttcctcccttcaacattctgtctcctcctacctagcatcttctttcttttagtctt
tctagaatgtccatctgtttttggccattgcggagagagaagctgagctttaaaggagtaggagcttcaaaggcgtaggagcttcaaaattcttgtttcttcatgtttgatcaccctt
ctaaacctgtcttctgttccttctgctattcttttttcttagagcataggaaaggggagcttttaaattaatacttaaagcatggaaaaaaagaacttgagaagaaagtaaaacaagg
gagatgaggctagtaaagtaaggaaaatgaagaggaagaggaggaagggttagcttctaaattccaagtcaaattgatatggaacaggcaagccgcttgtcttacttaaactt
cagaaaaggatctgctgaaacttgatagaaatggaaagggaaatccttggggggggaacctccaaacattagtaatgatattgaacaactcaaagtattgaggaaatctgca
ggctacatgcctgaagattacccatgcagatagaccaaaaggattagaattatctgttgatattagtaatatttattgacatctagctagtattggtaattttaagttttagattaatttct
ttggtaatagctatgatatattttatagacaagaattatatctataggcttgctatcataggctcttttaatcagcattaatttagtctactgatttttagcacatttgaatcattcacttatg
ctaggtaactcattgcaaaataaaaagatgattcctgtatgtatggcagctatacattaaggaggagtctaccagaatatgaaaaagtcagctgacctaaatattgctgagacaa
aggaaaacccactcccttggaggagcatgaccttttcctgtaattcttcccactgctgttgttgagctccttggatcctggctcctggacaccatcatcaagaagactttatggatg
ggctgtccacccactgagagaagaggagcatcagctacagtttctctctagattgccttcttcattttgagtaatgactgtcagcagggtcagattaaacacaaaacaactggac
aattgcttggaggactaaactataagggcactaacatgtcaatagtaggctaacacatccatggaaaatatatttaccagctcttctctcagggaggattctgtgtggggttgga
agtaatgatttgttaaattccttaggggtagaaagtagggcataatcagaatatagaggaatatgctgtttgacttcagggtttctgtttttcttactaggatatataaaacagggact
ctagctagattgtttatgaccacagagggtaggctgagtgctcccatgatcttcctgcttggttcttgcccatacagaggtcagcctttcctctaataaagattgaacaagtagtggtctgaggga
mouse-Evf2
(SEQ ID NO: 161)
gcttcaaattggatggcactgcagctggaggctttgttcagaattgatcctggggagctacgaacccaaagtttcacagtaggaagggggaaaaaagaaaagaaa
acatttttcctaatgtaacaatgcgaatgctagaaaatgacaagactgatcggttttaaaccattctgaagactgactgagcgtggaagttgctcaacaaaaaagggaacgggg
atattgaaccagagagaaacctacgcccagaagaacatgtccctggattgctttcccactgctgtggagtgtcttgaacactggtccctggacaccaacttcaagaagacttca
tggatggctgtccagtcttatgagccacagtttcccctctacattttcttcactccagcgaggctcttatcagggtcagatcagagatgaaccagctggacgacagattggagcg
ctgacctcttagagtgctaacagtgaacagtgtggggtcagatctatagaaagataataataagaaaacaccctatatgcaagggagagggatggttcataatttcttaaagatt
gaaatcaaggaacaatcaaaatatagaagaatgtggacgtgttttgctgcaggacttctgttttgtccccattggaatatgtattatggtattcctgttggatcaggactcaggggc
aaggctaagcattccagtggtcctcctacttagctcttgtcctttcgtaagaaacaccaactcattagtctctatattacttctctgtactgtagatctgcattcttgatctgagagatat
tggcaatgacactcttgtatgataaagctcaatgataagagtacttcaaacccccttgaactttttgtttatacatcaagtggtgacattgtgtattgagctaattagatcaatggagt
cacagggtgatactgaactcttttaaaatatttggctgaaacatgacattgtagttatttgtagaagagaacattatggaatatgaaaaacatcacagaacacagaactagcagc
agaaactagcagcaggtagacatttttccttttccatagagctttcaaccaaatgtctctgtagaaaatagtggctatcgtgtatatatatagccacatagatgtccttgagtgtacc
ctgtagtcagtgggagagttcctactgccacagtcatggccatggctatgttctctaagcctacattttataaacactctgtgaatcttgactacttttctttagcaagcattgcaaag
tcctgggatgtcagagaagtgcctggggttggcagggtttctagagaggaaattgttaaatgatttgaaccagaaaacaaacaggggatggggttcagaaccaacaattacc
tctattctatgtaggaaaccacaacatgaaatatgctgggcatggaaactttgataccttggtttttcattctttttaaaaattaatactaaagagctatgcgactgtaggcaagccat
ttcccatcccctgtgaatatctcccagatgactttaaatcccttctagttctgaaaggcttttaacatcagggcccaggctccagtggccagtttcaaaataccctcccttttgatgtt
aggttacataaacattgttcttttttagggagggtctcttttatcaacttttaaaaacacacatcaggttctctggtattaaaaagatgccatctctgagtcccctactatctgtgctgcc
tgcctttcctcctgttctttccttattcccatccctattgaacttgtgctatgcagtatgcatcaggtatgtgttagctttggggatacatgatagataaactggacacacagggtcttc
ccattctcttctggaattttctttggagggagcctcttgtatctagacagaccgtgctgtggtaccccagaggtaaccacctacaggcttcactctgcctaagcaattttgctgtgc
actaagatacacattcaagtaactttagattaccacaataactttctccaggtatgaggaaaagagataatttacttctgagatgtgtataggatagccctccatcctgggaagaa
cagtgactactccctgcatcccgaccttgcccagggaaagctaatgtttctctgtgttatccctgtgacttgccacttctttaaaaaggaatgggcaaacaataaacagacaaaa
atgttgtctgacctcattggaaatccttttaagaattaatcctttctatctccttcattatcaacaaatctattgaatacttatctctgagtccagggcatattttataatacataaaacaat
ggaatttcaaaattggagcactgacatacaatattggttttgagtatttttattatagggaatgactttagacattgcaatttatgacttaactgataaaatggatgactcttgactttca
attttcattttcagttcagtcgaggaatagcttcctccaggtaatgtctatactttcctatgactaagggctctaactatctctgttgcttttctttatgtaggcatatgttagtatttattttc
tatatgacaaatgtattaaagaaagcatgaaattaatgagataaacttttcagataggagtttagaaaatcaaggggccaagataaataaatgaaaaatcaacttaaataattaac
atattccagatatattggaataaatgtttattgtacccttttggttttgtcttgggttatttttttcttatctcactgatttttttttctttcctttttagcttttttgtcttttttgatttttgttgttgcgtttctcctttt
tttttttcttgttgatgttgtttgtttgtttgtttgtttgttttttgagaaagaacagaaggttggttggatagggaggtggggaagatctatctggatggagttgggaggag
ggaaaatacacgatcaaaatatattttgtgatgggcagggcatagtggtacatgtctttaatctcagcactctggaggcagaggcaggtggatctctatgagatggaggctagc
ctgatatacaaagtgagaccagaacatagggctgcctcaaaaactttatatatatattaaaaatgtttgctttttgagacagtcacagataaccaaaactgatcttgtaatgatgtaa
acatgtccagctaattttcaaatattgtagggcagcatttctccctttgtgcacacgtggagtcagcaaatccatataattctaaccattctggtgaaaaggagaacactcggcca
agcatctcacacttccaagtgtgaagccttgtttgaaagctccgagtatctaaatagtagccctgtgaaaggtaaatttatgaatggtctggtgtgttcttattccagccattgacct
taaagcaacttatatatgttttctttatccttcaagagaaaagaaaaatcatatttttccaagcaattaaaattcttctgcttcaggtaggaagaaggaattaggagttatgtctccttgt
atataattgcaagtttcatttttcttgttttaatgattgacagaaaactgataaactgagacatctccttattagggttgaatgtactctcttggtggccccattgctaatttgtttgactat
tttccatgatttcttactctgtaatggaaaggtttattaaatatgagggttgcaaagctttctgaatactaatgaacttatttgccaaaatttaaatgttcttcttgtcagtgaatgcctgtc
tcacttaacaggcaccaaattgaataatgaagaaaattagactctatcgtaccctcaagagaaatcgcgtgtgaattgtaatagaaaattgagggagaaaagggtcatattgta
gcaataacactagataatttggattattttaaaaaaggatgaacttagggaagctcaggtcttttcaaagaaacacacatttggttaattcatgcaaaacgctggtttcccctcaac
ccaggtggtctatacctatcgccagtttacagaaaaaggaagccaggtggatggaaaacgtgtgccaagtttctgtgcttacaatccactaaactcattctcatatgaggacttt
atatacctgtgatggagtgggaaaatcaataacctggaaaaaatgagtaccattttccaaagaagttcaataaagagatggaatttgggaaactgctgcagttcttcctataagcataaaaaaaaaaaaaaaaa
human-IncRNA N1
(SEQ ID NO: 152)
attccggcgtggagttctctgagttcggtggcacttgggatattccacttcagtgccatgggaagaaaatggtaagcaaaaagcaaaaggaataatgcaaaaaaaa
aaaaaaaattataatgttgtggtatccaattgctcctgcttcagccttttatccaaagccttgagatggatctgcctcatccaggaagttttcctgcttcgttagagtatcttcgttttctt
tatcagacgctggctggcactccacgccagtgcacggctccatggaatgcctgctgttggacacttgcagagctctgccaccaagcaacctcctgcattggaaatcggggg
actactccaggatcttatgcgtgcttgctgtttgaagctgcagaaaagtgaataatcaattaagtattgatttcttgtaagatttggtaaatttcaaaggcaagacaaagaggatgg
cttaaggaaacactgcagggacttggcgtaaattagacaactaacggaagcgcagaataaaataacacctcaactgaagatttccttggaagaattagcactagctattggaa
gacaatggtggatttaatcaaaacagcaggcatgcaagaattttacccgtgaaccagaaaaaggagctaattataaccacctttttaatggagctaatttgactgaatggatgca
aatgtgctttatttatggatgaagaagcaagccttactgcatgatcttgggatcatcggatctgctaagctttgcagctcctgttaccttcagtacattttttatttccggagaaggtat
ttgtatgtaaacaacaactaaccatgcaaaagattcatcggaacaatatactgtggtatatgagatataatcataattttgcagcctctcaggatttcctctaacttggaggttttgca
acctttgtgtgcaatgggttatggtaaacaataaatgaatgatgctaaa
human-IncRNA N2
(SEQ ID NO: 153)
ggggagccgccggagggcagctggggctgcggaattttggaagcgcagaagttttctcctcctgtcctgtaactggttttcactcactcaactgagagatttctttgt
tgcaatgatggggtctaagataacacttctggaggctgcaggcgagaaacacagctgatatcatcttttattgtgtgttgttatttgcctagcattataaattaaggaggaatagta
acaaagagcttgaagacatgcacagctcacaggccccgggtggaggctggcgacatcagacagacagaaccaagacatctgaggggcaaccaggaggtgcgtgtggc
tgcagagcacacagacttgtctttggacaaaattgagaagaactcataaacttggcttcctcgcttctgctcctcagacgcgcgtggatgatttgaagatattagcctaacaaga
tggaagcaggtttaaaattttacataagaagttacctttttcagttgtgttgatgttcctttcagctggtcctatagtacccctcctcaggaatgtctccccagtgcaaggacaaaga
ctgaagagactgctatattgatggactctcaagccaactatgaagttgaaacaaagaaagtgatcacctgaagacacctcctctgctaagaaacacccccaaattgtgcagctt
ctgccactagaactctcagaacaagagacaatcttttcaagaaacagaaaaactcaataatgacatctagattttcatgagccaagaactttcccttcctcatgtgtattcctctgtt
tgtacttaaattcatgtgacattcatttttttcctagtatggatatgcttattaatgcacttgtttcaaaatcccaaattgcacaaatgtgttaatattttaagaaacaaaatgaatcctaca
aggagaatgatttttagccacacatagggttggatcttgagagtgacctacagaataaaagtacttttaaaataaagtagtcagaggctattcaaagggtaaaataatcatagta
ccacattggtccacttgacactaaccaatcgatcatttttttttaatcaagaaagctagattctatcagataaaatcactgcttctaaagagtttaaatctagttagaaaaagttataga
aatgtttgcaaagataagtaacagatagagtcagtagaggataagatcaaaaacaaaaccaagcaaaagatgagttcaggggagtttgccatcaagttggcaaaactgactt
acttagggaagaaagttataaaacaggaaaatatgagatgaaccttgagtgatgtggaagatttagataaatggaaaggaaggagaaaatggagttctttaggtggttgtaatt
ggaggaggaaatgaatacacacatcttgttgacttaaacccagacattcagcagctctctatacatatctggaaaagactgcacagtcacctcctgtctctcaccccaggtatta
cttagaattattatcatatttcccttcctttaaagtaagtaagggtgattggtgacaatatggagaactatgatttttccattaacctaataataattggtatttattgagttctgttaagca
ttttacatattaactcacttaagcctttcaacagccttgcaaaataggtattattatccccattttacaggcaagaaaactgaggtttaagtaacttgccgaagtgccatatacaggg
ctcacattcagtattgcagttgcaaagctcatgatctatagtgccaagttgcaatattgtagtcaatgtcacaattattacccctttttatattccttgatatttttccatggcaaacaatt
agctatttcatttaataatcacctaaaacttttcagtcttctgattaaaattacgctggagtgatagaatgtattttcatgatagaaattgggaaaaaaaatggggaatgaagtttatca
gcatttcagacttgttttttttttttttttttgcaagactttgatgagattgttcacttttgtctatgtaaaatcccaaatccttgagaataaaaaagggggaggtttaagtcacttgttgcaa
tgccctttttaatagaggcaataaatctaaaggccataaatttagagtgacttacagaagatcgaactttggagtgtggcagagtaagggatggaaaccgggccctccagttca
ctatcagtagcttttgcactggtctgcccttcctaaattaagtatgcacttcaatttgatgagtggaaacagtctatctgggcagtaaccagggagctttgtgcctagtagattgctt
ctgttctgcacttctttggtttcccacctcaatgtaaaaaatagctagcaatgaagtccagaagttgtcaatggttcatccccagaagaatgcataatgtccaaagttgtatgtgtat
gatgtcttcaatggtattaagttatttcaaattcttagttcacctacataaatcatttctaacaagcatcttcttaaccaactttatgcacagtgtatgtttgtaagtgcttctgcacgaat
gtttatacatgactgtttccatagtacttatgtttttaaaaatattcagtcatttcctactataatcctcatgtatccatgtaactgactcaaaaatacttcagccacagaaagctaaaact
gagcaaatctcattcttcttttccatcccctttgcatgtggctggcatttagtaatgattaataatatggccagctgaataacagaggtttgagacacaattctttctcaaaggagtca
gctaagctgggtctacttatggacaaacatctaaatgtgtggaagtatctgatatttgacaatggtaaatttccacttagctagctagcattgtcagacttcaatctcctcatggctct
ggccgtcctgttttaagcatgataattgttggccacatctcacatagttctcattgagtgagttcataaataaacagggtttttttttttttttaaagagcagccaagcacaaagtgtga
ctttgttgacattttatgtgactttgtcatatgttcctaacccccaataaaagcaatgttgcatcaactgtgaa

### EXAMPLES

The present disclosure will be further elaborated below in conjunction with specific embodiments. It should be understood that these examples are only for illustrating the present disclosure and are not intended to limit the scope of the present disclosure.

### Example 1. Trans Differentiation of Glial Cell to Neuron Induced by Knocking Down Pnky

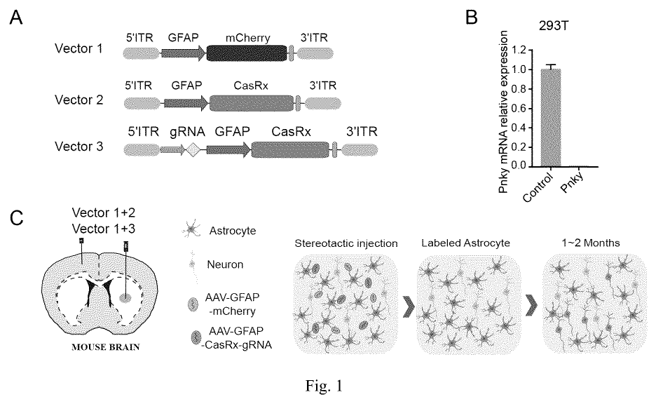

In order to study the efficiency of CasRx in knocking down Pnky, we transfected the plasmids of the control group and the experimental group into 293T cells respectively. The Q-PCR results showed that CasRx could efficiently knock down the expression of Pnky in human cells, with the Pnky expression in the experimental group only accounting for 0.5% of the control group (FIG. 1A). In order to further use CasRx to edit Pnky in vivo, we constructed an AAV expression system. The system uses the glial cell-specific promoter GFAP to promote the expression of mCherry to specifically label astrocytes, and at the same time use GFAP to promote the expression of CasRx to achieve specific expression of CasRX in astrocytes (FIG. 1B). Different AAV combinations were injected into the mouse brain. The control group was injected with GFAP-mCherry+GFAP-CasRx, and the experimental group was injected with GFAP-mCherry+GFAP-CasRx-gRNA(Pnky). in which GFAP-mCherry marked astrocytes. GFAP-mCherry was used to label astrocytes, and the samples were analyzed 1-2 months after injection (FIG. 1C).

### Example 2. miRNA and lncRNA-Mediated Transdifferentiation of Glial Cells into Neurons

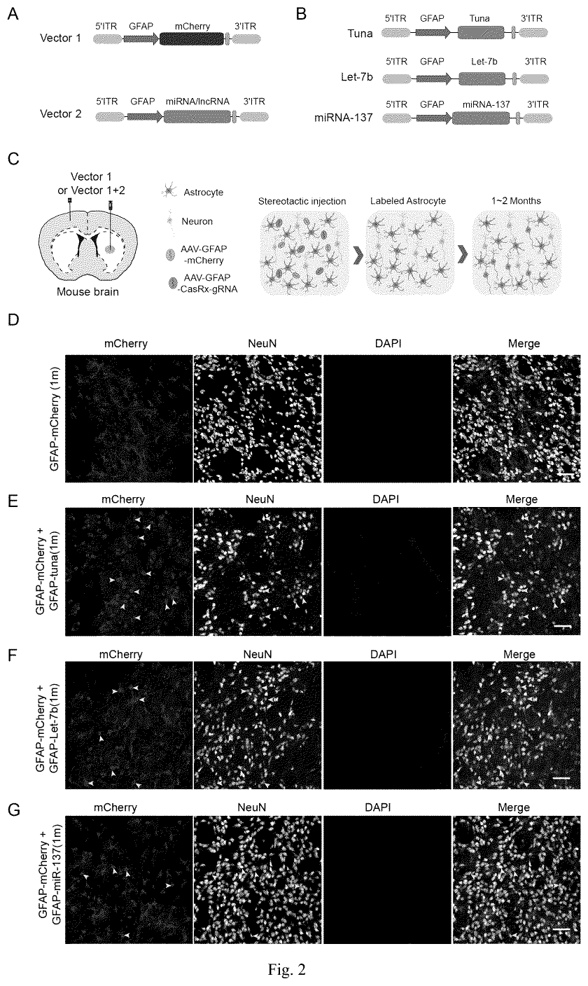

To explore whether miRNAs and lncRNAs can transdifferentiate glial cells into neurons in vivo, we constructed an AAV system that use AAV-GFAP-mCherry to label glial cells and use the glial cell-specific promoter GFAP to promote the expression of miRNAs/LncRNAs in glial cells (FIG. 2A). To explore the functions of Tuna, Let-7b and miRNA-137, we constructed AAV expression vectors of AAV-GFAP-Tuna, AAV-GFAP-Let-7b and AAV-GFAP-miRNA-137 respectively (FIG. 2B). AAV was injected into the striatum of the mouse brain, analyzed after 1-2 months of injection, and immunofluorescent staining was performed with the neuron-specific marker NeuN. If the glial cells labeled with GFAP-mCherry were transdifferentiated into neurons, GFAP-mCherry would be co-labeled with NeuN (FIG. 2C). In the control group, the results of 1 month after injection of GFAP-mCherry showed that glial cells still maintained the typical characteristics of glial cells and were not co-labeled with the neuron-specific marker NeuN (FIG. 2D). However, in the GFAP-Tuna group, the red fluorescently labeled cells exhibited clear neuronal morphology with rounded cell bodies and some longer and slender processes. Further staining with the neuron-specific marker NeuN found that red cells overlapped with NeuN, suggesting that these red cells had transdifferentiated from glia to neurons (FIG. 2E). Similarly, in the GFAP-Let-7b group and GFAP-miRNA-137 group, some red cells exhibited typical neuronal morphology and co-labeled with the neuron-specific marker NeuN. Meanwhile, we also observed in the GFAP-Let-7b group and GFAP-miRNA-137 group that some red blood cells still maintained in glial cell morphology, which indicated that some glial cells transdifferentiated into neurons, and some cells did not transdifferentiate and remained as astrocytes (FIGS. 2F and G).

### Example 3. Transdifferentiation of Glial Cells into Neurons Induced by Overexpression of miRNA or lncRNA

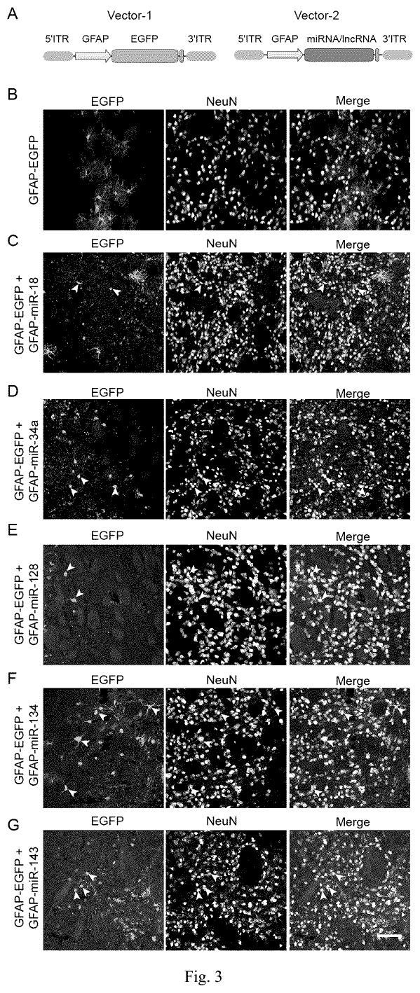

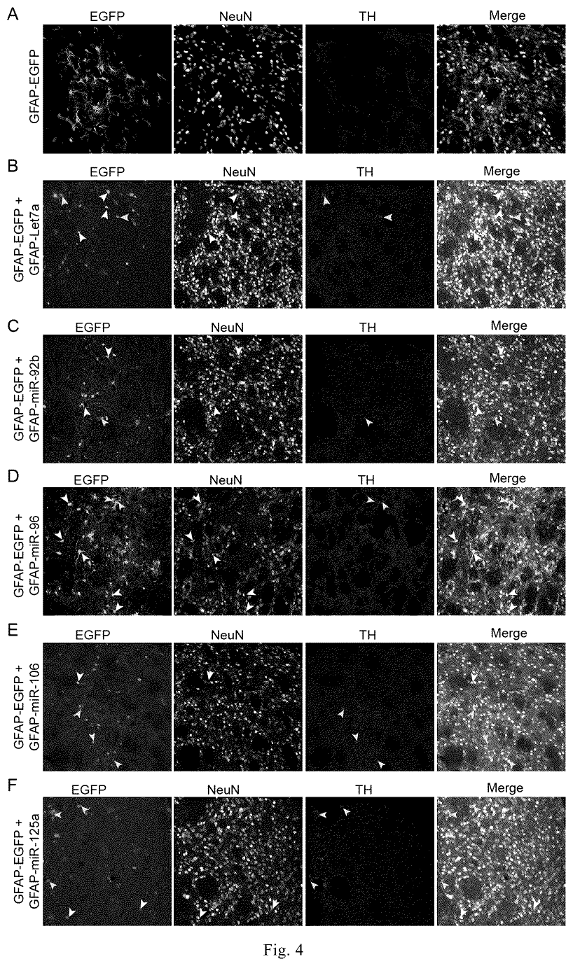

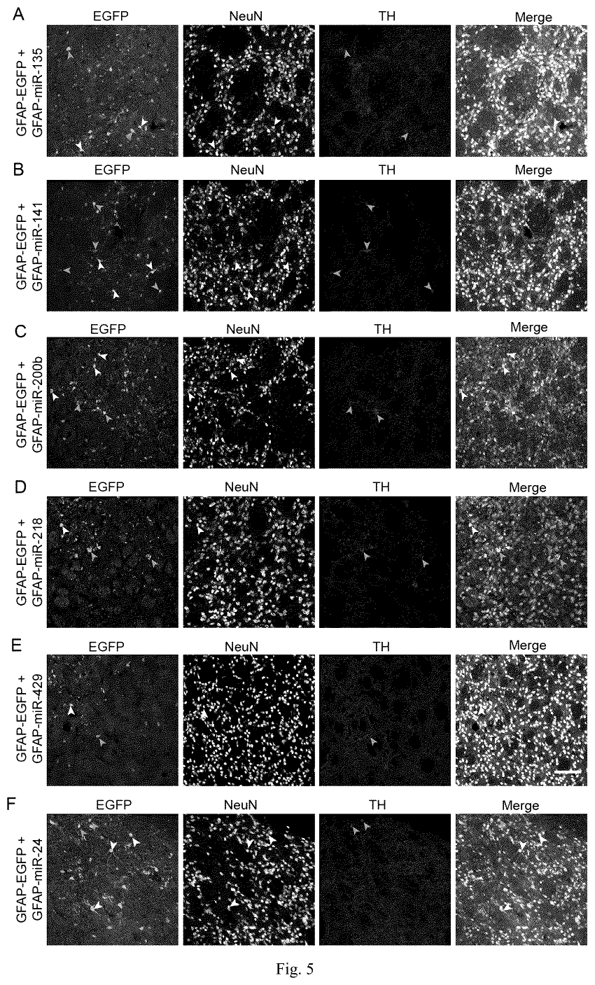

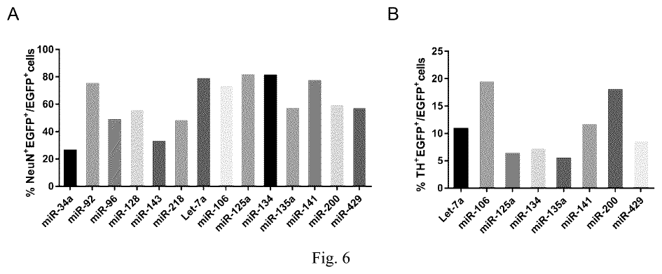

In order to further study whether the miRNA such as miR-18b, miR-24, miR-34a, miR-128, miR-134, miR-143, Let-7a, miR-92b, miR-96, miR-106, miR-125a, miR-135, miR-141, miR-200, miR-218, miR-429 could transdifferentiate astrocytes into neurons, we constructed the expression vectors of miR-18b, miR-24, miR-34a, miR-128, miR-134, miR-143, Let-7a, miR-92b, miR-96, miR-106, miR-125a, miR-135, miR-141, miR-200, miR-218, miR-429 which were driven by the astrocyte-specific promoter GFAP and packaged them into AAV. Each group of AAV was mixed with AAV-GFAP-EGFP and then injected into the striatum of mice, wherein AAV-GFAP-EGFP could specifically mark the astrocytes in the striatum as green. After 1-2 months of injection, it was found that the cells marked with green fluorescent signal still in the control group still maintained the typical astrocyte morphology, while in the miRNA overexpression groups including miR-18b, miR-24, miR-34a, miR-128, miR-134, miR-143, Let-7a, miR-92b, miR-96, miR-106, miR-125a, miR-135, miR-141, miR-200, miR-218, miR-429, a large number of AAV-GFAP-EGFP-labeled astrocytes were found to have transformed into neuronal morphology (FIGS. 3B-G, FIGS. 4A-F and FIGS. 5A-F). Further staining with neuron-specific protein markers, it was found that most of the cells with typical neuronal morphology had expressed NeuN although some cells expressed NeuN weakly (FIGS. 3B-G, FIGS. 4A-F and FIGS. 5A-F). In order to further study whether it can transdifferentiate into dopaminergic neurons, we performed immunofluorescence staining with dopamine neuron-specific protein marker TH. In the miRNA overexpression groups, TH positive cells were observed in the group including Let-7a, miR-92b, miR-96, miR-106, miR-125a, miR-135, miR-141, miR-200, miR-218, or miR-429. Among these groups, miR-106, Let-7a, miR-141, and miR-200 exhibited a higher number and proportion of TH-positive cells (FIG. 6). This indicated that overexpression of miRNA such as miR-18b, miR-24, miR-34a, miR-128, miR-134, miR-143, Let-7a, miR-92b, miR-96, miR-106, miR-125a, miR-135, miR-141, miR-200, miR-218, or miR-429 can transdifferentiate astrocytes into neurons. Moreover, overexpression of Let-7a, miR-92b, miR-96, miR-106, miR-125a, miR-135, miR-141, miR-200, miR-218, miR-429, or miR-24 not only can transdifferentiate astrocytes into neurons, but also can transdifferentiate them into dopaminergic neurons.

All documents mentioned in this disclosure are incorporated by reference in this application as if each were individually incorporated by reference. In addition, it should be understood that after reading the above teaching content of the present disclosure, those skilled in the art may make various changes or modifications to the present disclosure, and these equivalent forms also fall within the scope defined by the appended claims of the present application.

1. A method of producing neuronal cells from non-neuronal cells, comprises transdifferentiation or reprogramming the non-neuronal cells into neuronal cells by enhancing the expression or activity of miRNA selected from Let-7a, Let-7b, miR-18a/b, miR-24-3p, miR-34a, miR-92b, miR-96, miR-106, miR-125a/b, miR-128, miR-134, miR-135, miR-137, miR-141, miR-143-3p, miR-184, miR-200, miR-218, miR-219, miR-228, miR-284, miR-429, miR-430, or enhancing the expression or activity of lncRNA selected from utNgn1, RMST, Tuna, Linc-Brn1b, Dali, Miat/Gomafu, NBAT-1, Malat1, Dlx1as, Six3os, Evf2, LncKdm2b, lncRNA_N1, ncRNA_N2, lncRNA_N3, or any combination thereof,
preferably, the expression or activity of the miRNA or lncRNA is enhanced through, for example, overexpression, gene activators, epigenetic modifications, miRNA mimics, direct delivery of RNA, small-molecule compounds, and/or RNA stabilizers.

2. (canceled)

3. A method for producing neuronal cells from non-neuronal cells, comprises transdifferentiation or reprogramming the non-neuronal cells into neuronal cells by reducing the expression or activity of miRNA selected from miR-7a, miR-15, miR-23a/b, miR-25, miR-29a, miR-129, miR-137, miR-138, miR-155, miR-195, miR-214, miR-222, miR-223, miR-132, miR-133, or reducing the expression or activity of lncRNA selected from Pnky, Paupar, HOTAIRM1, IncR492, TUG1, or any combination thereof,
preferably, the expression or activity of the miRNA or lncRNA is reduced by techniques such as DNA editing or RNA editing induced by gene editing technology, RNA expression inhibitors, antisense oligonucleotides, small RNA interference, miRNA technology, small molecule compounds, genes inhibiting technology, and/or epigenetic regulation;
more preferably, wherein RNA editing includes CRISPR-mediated RNA degradation or RNA translation inhibition, RNA single base editing, insertion or deletion of bases of RNA, alteration of RNA splicing, or RNA epigenetic modifications.

4. (canceled)

5. (canceled)

6. The method according to claim 1, wherein the miRNA selected from Let-7a, Let-7b, miR-18a/b, miR-24-3p, miR-34a, miR-92b, miR-96, miR-106, miR-125a/b, miR-128, miR-134, miR-135, miR-137, miR-141, miR-143-3p, miR-184, miR-200, miR-218, miR-219, miR-228, miR-284, miR-429, miR-430, or the miRNA selected from miR-7a, miR-15, miR-23a/b, miR-25, miR-29a, miR-129, miR-137, miR-138, miR-155, miR-195, miR-214, miR-222, miR-223, miR-132, miR-133, or the lncRNA selected from utNgn1, RMST, Tuna, Linc-Brn1b, Dali, Miat/Gomafu, NBAT-1, Malat1, Dlx1as, Six3os, Evf2, LncKdm2b, lncRNA_N1, lncRNA_N2, lncRNA_N3, or the lncRNA selected from Pnky, Paupar, HOTAIRM1, IncR492, TUG1, are homologous miRNA or homologous lncRNA from different species.

7. The method according to claim 1, wherein the non-neuronal cells comprise, for example, glial cells, fibroblasts, stem cells, neural precursor cells, neural stem cells, wherein the glial cells are selected from astrocytes Glial cells, microglia, oligodendrocytes, ependymal cells, Schwann cells, NG2 cells, satellite cells or any combinations thereof, preferably comprise astrocytes;
preferably, wherein the glial cells are derived from the brain, spinal cord, eyes or ears, wherein glial cells in the brain are derived from the striatum, substantia nigra, ventral tegmental area of the midbrain, spinal cord, hypothalamus, dorsal midbrain, or cerebral cortex, more preferably are derived from striatum or substantia nigra;
even more preferably, the non-neuronal cells are glial cells.

8. (canceled)

9. The method according to claim 1, wherein the neuronal cells are preferably dopaminergic neurons, GABA neurons, 5-HT neurons, glutamatergic neurons, ChAT neurons, NE neurons, motor neurons, spinal cord neurons, spinal motor neurons, spinal sensory neurons, pyramidal neurons, interneurons, medium spiny neurons, Purkinje cells, granule cells, olfactory sensory neurons, periglomerular cells or any combinations thereof, more preferably are dopaminergic neurons,
more preferably, the neuronal cells are dopaminergic neurons.

10. (canceled)

11. (canceled)

12. The method according to claim 1, wherein the miRNA or lncRNA is Let-7a, miR-92b, miR-96, miR-106, miR-125a, miR-135, miR-141, miR-200, miR-218, miR-429, or miR-24, the non-neuronal cells are glial cells, and the neuronal cells are dopaminergic neurons.

13-16. (canceled)

17. The method of claim 1, wherein enhancing the expression or activity of the miRNA, lncRNA, or a combination thereof comprises:
(a) exogenously expressing of the miRNA or lncRNA or a combination thereof, for example, exogenously expressing was achieved by an expression vector comprising a promoter;
(b) delivering the miRNA or lncRNA or a combination thereof in the form of DNA or RNA into the cell;
(c) activating the endogenous expression of the miRNA or lncRNA or combination thereof, such as gene expression activators and epigenetic regulatory elements, etc.; or
(d) delivering an analog or agonist of the miRNA or lncRNA or combination thereof to the cell;
wherein, it is preferable to express the miRNA or lncRNA or a combination thereof exogenously, for example, through an expression vector containing a promoter.

18. The method of claim 1, wherein the expression or activity of the miRNA or lncRNA or combination thereof is reduced through the use of: antibody, small molecule compound, microRNA, siRNA, shRNA, antisense oligonucleotide, binding protein or protein domain, polypeptides, nucleic acid aptamers, gene editors, epigenetic regulatory elements, transcriptional repression elements, or any combinations thereof.

19. (canceled)

20. Pharmaceutical composition or pharmaceutical kit or reagent kit, which comprises reagent that enhance the expression or activity of miRNA selected from Let-7a, Let-7b, miR-18a/b, miR-24-3p, miR-34a, miR-92b, miR-96, miR-106, miR-125a/b, miR-128, miR-134, miR-135, miR-137, miR-141, miR-143-3p, miR-184, miR-200, miR-218, miR-219, miR-228, miR-284, miR-429, miR-430, or lncRNA selected from utNgn1, RMST, Tuna, Linc-Brn1b, Dali, Miat/Gomafu, NBAT-1, Malat1, Dlx1as, Six3os, Evf2, LncKdm2b, lncRNA_N1, lncRNA_N2, lncRNA_N3 lncRNA, or reagent that enhance the expression or activity of any combination thereof; or a reagent that reduce the expression or activity of miRNA selected from miR-7a, miR-15, miR-23a/b, miR-25, miR-29a, miR-129, miR-137, miR-138, miR-155, miR-195, miR-214, miR-222, miR-223, miR-132, miR-133, or lncRNA selected from Pnky, Paupar, HOTAIRM1, IncR492, TUG1, or reagent that reduce the expression or activity of any combination thereof.

21. The pharmaceutical composition or pharmaceutical kit or reagent kit according to claim 20, wherein the reagent that enhances the expression or activity of the miRNA or lncRNA or a combination thereof is selected from: an expression vector, the miRNA or lncRNA or a combination thereof in the form of DNA or RNA, an endogenous activator of the miRNA or lncRNA or a combination thereof, an analog or agonist of the miRNA or lncRNA or a combination thereof.

22. The pharmaceutical composition or pharmaceutical kit or reagent kit according to claim 21, wherein the expression vector is a gene therapy vector, preferably is a viral gene therapy vector, more preferably the viral vector is selected from: adeno-associated virus vector, recombinant adeno-associated viral vector, self-complementary AAV, adenovirus vector, lentivirus vector, retrovirus vectors, herpesvirus, SV40 vector, poxvirus vector, and any combinations thereof, wherein the viral vector is preferably selected from AAV and rAAV.

23. The pharmaceutical composition or pharmaceutical kit or reagent kit according to claim 20, wherein the reagent that reduces the expression or activity of the miRNA or lncRNA or combination thereof, is selected from: antibodies, small molecule compounds, microRNA, siRNA, shRNA, antisense oligonucleotides, binding proteins or protein domains, polypeptides, aptamers, gene editors, epigenetic regulatory elements, transcriptional repressor elements, or combinations thereof.

24. The pharmaceutical composition or pharmaceutical kit or reagent kit according to claim 20, further comprising a carrier or vehicle for delivering the reagent,
preferably the vector or vehicle is a viral vector, liposome, nanoparticle, exosome, virus-like particle, preferably is AAV.

25. (canceled)

26. The pharmaceutical composition or pharmaceutical kit or reagent kit according to claim 20, wherein the composition is locally administered to at least one of the following: i) glial cells in the striatum; ii) glial cells in the ventral tegmental area; iii) glial cells in the substantia nigra; iv) glial cells in the hypothalamus; v) glial cells in the spinal cord; vi) glial cells in the prefrontal cortex; and vii) glial cells in the motor cortex,
preferably wherein the pharmaceutical composition or pharmaceutical kit or reagent kit is formulated for cell transfection, cell infection, endocytosis, injection, intracranial administration, intraocular administration, inner ear injection, inhalation, parenteral administration, intravenous administration, intramuscular administration, intradermal administration, epidermal administration, or oral administration.

27-29. (canceled)

30. The method according to claim 2, wherein the miRNA selected from Let-7a, Let-7b, miR-18a/b, miR-24-3p, miR-34a, miR-92b, miR-96, miR-106, miR-125a/b, miR-128, miR-134, miR-135, miR-137, miR-141, miR-143-3p, miR-184, miR-200, miR-218, miR-219, miR-228, miR-284, miR-429, miR-430, or the miRNA selected from miR-7a, miR-15, miR-23a/b, miR-25, miR-29a, miR-129, miR-137, miR-138, miR-155, miR-195, miR-214, miR-222, miR-223, miR-132, miR-133, or the lncRNA selected from utNgn1, RMST, Tuna, Linc-Brn1b, Dali, Miat/Gomafu, NBAT-1, Malat1, Dlx1as, Six3os, Evf2, LncKdm2b, lncRNA_N1, lncRNA_N2, lncRNA_N3, or the lncRNA selected from Pnky, Paupar, HOTAIRM1, IncR492, TUG1, are homologous miRNA or homologous lncRNA from different species.

31. The method according to claim 2, wherein the non-neuronal cells comprise, for example, glial cells, fibroblasts, stem cells, neural precursor cells, neural stem cells, wherein the glial cells are selected from astrocytes Glial cells, microglia, oligodendrocytes, ependymal cells, Schwann cells, NG2 cells, satellite cells or any combinations thereof, preferably comprise astrocytes;
preferably, wherein the glial cells are derived from the brain, spinal cord, eyes or ears, wherein glial cells in the brain are derived from the striatum, substantia nigra, ventral tegmental area of the midbrain, spinal cord, hypothalamus, dorsal midbrain, or cerebral cortex, more preferably are derived from striatum or substantia nigra;
even more preferably, the non-neuronal cells are glial cells.

32. The method according to claim 2, wherein the neuronal cells are preferably dopaminergic neurons, GABA neurons, 5-HT neurons, glutamatergic neurons, ChAT neurons, NE neurons, motor neurons, spinal cord neurons, spinal motor neurons, spinal sensory neurons, pyramidal neurons, interneurons, medium spiny neurons, Purkinje cells, granule cells, olfactory sensory neurons, periglomerular cells or any combinations thereof, more preferably are dopaminergic neurons; more preferably, the neuronal cells are dopaminergic neurons.

33. The method according to claim 2, wherein the miRNA or lncRNA is Let-7a, miR-92b, miR-96, miR-106, miR-125a, miR-135, miR-141, miR-200, miR-218, miR-429, or miR-24, the non-neuronal cells are glial cells, and the neuronal cells are dopaminergic neurons.

34. The method of claim 2, wherein enhancing the expression or activity of the miRNA, lncRNA, or a combination thereof comprises:
(a) exogenously expressing of the miRNA or lncRNA or a combination thereof, for example, exogenously expressing was achieved by an expression vector comprising a promoter;
(b) delivering the miRNA or lncRNA or a combination thereof in the form of DNA or RNA into the cell;
(c) activating the endogenous expression of the miRNA or lncRNA or combination thereof, such as gene expression activators and epigenetic regulatory elements, etc.; or
(d) delivering an analog or agonist of the miRNA or lncRNA or combination thereof to the cell;
wherein, it is preferable to express the miRNA or lncRNA or a combination thereof exogenously, for example, through an expression vector containing a promoter.

35. The method of claim 2, wherein the expression or activity of the miRNA or lncRNA or combination thereof is reduced through the use of: antibody, small molecule compound, microRNA, siRNA, shRNA, antisense oligonucleotide, binding protein or protein domain, polypeptides, nucleic acid aptamers, gene editors, epigenetic regulatory elements, transcriptional repression elements, or any combinations thereof.


SEQUENCE LISTING (153 sequences)

SEQ ID NO: 1 - DNA, 22 bp, synthetic construct
  source 1..22: mol_type = other DNA
tgaggtagtaggttgtatagtt

SEQ ID NO: 2 - DNA, 21 bp, synthetic construct
  source 1..21: mol_type = other DNA
ctatacaatctactgtctttc

SEQ ID NO: 3 - DNA, 21 bp, synthetic construct
  source 1..21: mol_type = other DNA
ctatacaatctactgtctttc

SEQ ID NO: 4 - DNA, 80 bp, synthetic construct
  source 1..80: mol_type = other DNA
tgggatgaggtagtaggttgtatagttttagggtcacacccaccactgggagataactatacaatctactgtctttccta

SEQ ID NO: 5 - DNA, 72 bp, synthetic construct
  source 1..72: mol_type = other DNA
aggttgaggtagtaggttgtatagtttagaattacatcaagggagataactgtacagcctcctagctttcct

SEQ ID NO: 6 - DNA, 74 bp, synthetic construct
  source 1..74: mol_type = other DNA
gggtgaggtagtaggttgtatagtttggggctctgccctgctatgggataactatacaatctactgtctttcct

SEQ ID NO: 7 - DNA, 22 bp, synthetic construct
  source 1..22: mol_type = other DNA
tgaggtagtaggttgtgtggtt

SEQ ID NO: 8 - DNA, 22 bp, synthetic construct
  source 1..22: mol_type = other DNA
ctatacaacctactgccttccc

SEQ ID NO: 9 - DNA, 83 bp, synthetic construct
  source 1..83: mol_type = other DNA
cggggtgaggtagtaggttgtgtggtttcagggcagtgatgttgcccctcggaagataactatacaacctactgccttccctg

SEQ ID NO: 10 - DNA, 23 bp, synthetic construct
  source 1..23: mol_type = other DNA
taaggtgcatctagtgcagatag

SEQ ID NO: 11 - DNA, 23 bp, synthetic construct
  source 1..23: mol_type = other DNA
actgccctaagtgctccttctgg

SEQ ID NO: 12 - DNA, 23 bp, synthetic construct
  source 1..23: mol_type = other DNA
taaggtgcatctagtgcagttag

SEQ ID NO: 13 - DNA, 71 bp, synthetic construct
  source 1..71: mol_type = other DNA
tgttctaaggtgcatctagtgcagatagtgaagtagattagcatctactgccctaagtgctccttctggca

SEQ ID NO: 14 - DNA, 71 bp, synthetic construct
  source 1..71: mol_type = other DNA
tgtgttaaggtgcatctagtgcagttagtgaagcagcttagaatctactgccctaaatgccccttctggca

SEQ ID NO: 15 - DNA, 22 bp, synthetic construct
  source 1..22: mol_type = other DNA
tggctcagttcagcaggaacag

SEQ ID NO: 16 - DNA, 68 bp, synthetic construct
  source 1..68: mol_type = other DNA
ctccggtgcctactgagctgatatcagttctcattttacacactggctcagttcagcaggaacaggag

SEQ ID NO: 17 - DNA, 73 bp, synthetic construct
  source 1..73: mol_type = other DNA
ctctgcctcccgtgcctactgagctgaaacacagttggtttgtgtacactggctcagttcagcaggaacaggg

SEQ ID NO: 18 - DNA, 22 bp, synthetic construct
  source 1..22: mol_type = other DNA
tggcagtgtcttagctggttgt

SEQ ID NO: 19 - DNA, 22 bp, synthetic construct
  source 1..22: mol_type = other DNA
caatcagcaagtatactgccct

SEQ ID NO: 20 - DNA, 110 bp, synthetic construct
  source 1..110: mol_type = other DNA
ggccagctgtgagtgtttctttggcagtgtcttagctggttgttgtgagcaatagtaaggaagcaatcagcaagtatactgccctagaagtgctgcacgttgtggggccc

SEQ ID NO: 21 - DNA, 22 bp, synthetic construct
  source 1..22: mol_type = other DNA
agggacgggacgcggtgcagtg

SEQ ID NO: 22 - DNA, 22 bp, synthetic construct
  source 1..22: mol_type = other DNA
tattgcactcgtcccggcctcc

SEQ ID NO: 23 - DNA, 96 bp, synthetic construct
  source 1..96: mol_type = other DNA
cgggccccgggcgggcgggagggacgggacgcggtgcagtgttgttttttcccccgccaatattgcactcgtcccggcctccggcccccccggccc

SEQ ID NO: 24 - DNA, 23 bp, synthetic construct
  source 1..23: mol_type = other DNA
tttggcactagcacatttttgct

SEQ ID NO: 25 - DNA, 22 bp, synthetic construct
  source 1..22: mol_type = other DNA
aatcatgtgcagtgccaatatg

SEQ ID NO: 26 - DNA, 78 bp, synthetic construct
  source 1..78: mol_type = other DNA
tggccgattttggcactagcacatttttgcttgtgtctctccgctctgagcaatcatgtgcagtgccaatatgggaaa

SEQ ID NO: 27 - DNA, 23 bp, synthetic construct
  source 1..23: mol_type = other DNA
aaaagtgcttacagtgcaggtag

SEQ ID NO: 28 - DNA, 22 bp, synthetic construct
  source 1..22: mol_type = other DNA
ctgcaatgtaagcacttcttac

SEQ ID NO: 29 - DNA, 81 bp, synthetic construct
  source 1..81: mol_type = other DNA
ccttggccatgtaaaagtgcttacagtgcaggtagctttttgagatctactgcaatgtaagcacttcttacattaccatgg

SEQ ID NO: 30 - DNA, 23 bp, synthetic construct
  source 1..23: mol_type = other DNA
cggggccgtagcactgtctgaga

SEQ ID NO: 31 - DNA, 21 bp, synthetic construct
  source 1..21: mol_type = other DNA
tcacagtgaaccggtctcttt

SEQ ID NO: 32 - DNA, 82 bp, synthetic construct
  source 1..82: mol_type = other DNA
tgagctgttggattcggggccgtagcactgtctgagaggtttacatttctcacagtgaaccggtctctttttcagctgcttc

SEQ ID NO: 33 - DNA, 22 bp, synthetic construct
  source 1..22: mol_type = other DNA
tgtgactggttgaccagagggg

SEQ ID NO: 34 - DNA, 23 bp, synthetic construct
  source 1..23: mol_type = other DNA
cctgtgggccacctagtcaccaa

SEQ ID NO: 35 - DNA, 73 bp, synthetic construct
  source 1..73: mol_type = other DNA
cagggtgtgtgactggttgaccagaggggcatgcactgtgttcaccctgtgggccacctagtcaccaaccctc

SEQ ID NO: 36 - DNA, 23 bp, synthetic construct
  source 1..23: mol_type = other DNA
tatggctttttattcctatgtga

SEQ ID NO: 37 - DNA, 22 bp, synthetic construct
  source 1..22: mol_type = other DNA
tatagggattggagccgtggcg

SEQ ID NO: 38 - DNA, 90 bp, synthetic construct
  source 1..90: mol_type = other DNA
aggcctcgctgttctctatggctttttattcctatgtgattctactgctcactcatatagggattggagccgtggcgcacggcggggaca

SEQ ID NO: 39 - DNA, 23 bp, synthetic construct
  source 1..23: mol_type = other DNA
acgggtattcttgggtggataat

SEQ ID NO: 40 - DNA, 23 bp, synthetic construct
  source 1..23: mol_type = other DNA
ttattgcttaagaatacgcgtag

SEQ ID NO: 41 - DNA, 102 bp, synthetic construct
  source 1..102: mol_type = other DNA
ggtcctctgactctcttcggtgacgggtattcttgggtggataatacggattacgttgttattgcttaagaatacgcgtagtcgaggagagtaccagcggca

SEQ ID NO: 42 - DNA, 22 bp, synthetic construct
  source 1..22: mol_type = other DNA
catcttccagtacagtgttgga

SEQ ID NO: 43 - DNA, 22 bp, synthetic construct
  source 1..22: mol_type = other DNA
taacactgtctggtaaagatgg

SEQ ID NO: 44 - DNA, 95 bp, synthetic construct
  source 1..95: mol_type = other DNA
cggccggccctgggtccatcttccagtacagtgttggatggtctaattgtgaagctcctaacactgtctggtaaagatggctcccgggtgggttc

SEQ ID NO: 45 - DNA, 22 bp, synthetic construct
  source 1..22: mol_type = other DNA
ggtgcagtgctgcatctctggt

SEQ ID NO: 46 - DNA, 21 bp, synthetic construct
  source 1..21: mol_type = other DNA
tgagatgaagcactgtagctc

SEQ ID NO: 47 - DNA, 106 bp, synthetic construct
  source 1..106: mol_type = other DNA
gcgcagcgccctgtctcccagcctgaggtgcagtgctgcatctctggtcagttgggagtctgagatgaagcactgtagctcaggaagagagaagttgttctgcagc

SEQ ID NO: 48 - DNA, 23 bp, synthetic construct
  source 1..23: mol_type = other DNA
ccttatcacttttccagcccagc

SEQ ID NO: 49 - DNA, 22 bp, synthetic construct
  source 1..22: mol_type = other DNA
tggacggagaactgataagggt

SEQ ID NO: 50 - DNA, 84 bp, synthetic construct
  source 1..84: mol_type = other DNA
ccagtcacgtccccttatcacttttccagcccagctttgtgactgtaagtgttggacggagaactgataagggtaggtgattga

SEQ ID NO: 51 - DNA, 22 bp, synthetic construct
  source 1..22: mol_type = other DNA
catcttaccggacagtgctgga

SEQ ID NO: 52 - DNA, 22 bp, synthetic construct
  source 1..22: mol_type = other DNA
catcttactgggcagcattgga

SEQ ID NO: 53 - DNA, 22 bp, synthetic construct
  source 1..22: mol_type = other DNA
cgtcttacccagcagtgtttgg

SEQ ID NO: 54 - DNA, 22 bp, synthetic construct
  source 1..22: mol_type = other DNA
taacactgtctggtaacgatgt

SEQ ID NO: 55 - DNA, 22 bp, synthetic construct
  source 1..22: mol_type = other DNA
taatactgcctggtaatgatga

SEQ ID NO: 56 - DNA, 23 bp, synthetic construct
  source 1..23: mol_type = other DNA
taatactgccgggtaatgatgga

SEQ ID NO: 57 - DNA, 90 bp, synthetic construct
  source 1..90: mol_type = other DNA
ccgggcccctgtgagcatcttaccggacagtgctggatttcccagcttgactctaacactgtctggtaacgatgttcaaaggtgacccgc

SEQ ID NO: 58 - DNA, 95 bp, synthetic construct
  source 1..95: mol_type = other DNA
ccagctcgggcagccgtggccatcttactgggcagcattggatggagtcaggtctctaatactgcctggtaatgatgacggcggagccctgcacg

SEQ ID NO: 59 - DNA, 68 bp, synthetic construct
  source 1..68: mol_type = other DNA
ccctcgtcttacccagcagtgtttgggtgcggttgggagtctctaatactgccgggtaatgatggagg

SEQ ID NO: 60 - DNA, 21 bp, synthetic construct
  source 1..21: mol_type = other DNA
ttgtgcttgatctaaccatgt

SEQ ID NO: 61 - DNA, 22 bp, synthetic construct
  source 1..22: mol_type = other DNA
atggttccgtcaagcaccatgg

SEQ ID NO: 62 - DNA, 22 bp, synthetic construct
  source 1..22: mol_type = other DNA
catggttctgtcaagcaccgcg

SEQ ID NO: 63 - DNA, 110 bp, synthetic construct
  source 1..110: mol_type = other DNA
gtgataatgtagcgagattttctgttgtgcttgatctaaccatgtggttgcgaggtatgagtaaaacatggttccgtcaagcaccatggaacgtcacgcagctttctaca

SEQ ID NO: 64 - DNA, 110 bp, synthetic construct
  source 1..110: mol_type = other DNA
gaccagtcgctgcggggctttcctttgtgcttgatctaaccatgtggtggaacgatggaaacggaacatggttctgtcaagcaccgcggaaagcaccgtgctctcctgca

SEQ ID NO: 65 - DNA, 21 bp, synthetic construct
  source 1..21: mol_type = other DNA
tgattgtccaaacgcaattct

SEQ ID NO: 66 - DNA, 22 bp, synthetic construct
  source 1..22: mol_type = other DNA
agagttgagtctggacgtcccg

SEQ ID NO: 67 - DNA, 21 bp, synthetic construct
  source 1..21: mol_type = other DNA
tgattgtccaaacgcaattct

SEQ ID NO: 68 - DNA, 22 bp, synthetic construct
  source 1..22: mol_type = other DNA
agaattgtggctggacatctgt

SEQ ID NO: 69 - DNA, 110 bp, synthetic construct
  source 1..110: mol_type = other DNA
ccgccccgggccgcggctcctgattgtccaaacgcaattctcgagtctatggctccggccgagagttgagtctggacgtcccgagccgccgcccccaaacctcgagcggg

SEQ ID NO: 70 - DNA, 97 bp, synthetic construct
  source 1..97: mol_type = other DNA
actcaggggcttcgccactgattgtccaaacgcaattcttgtacgagtctgcggccaaccgagaattgtggctggacatctgtggctgagctccggg

SEQ ID NO: 71 - DNA, 21 bp, synthetic construct
  source 1..21: mol_type = other DNA
gtcttaccagacatggttaga

SEQ ID NO: 72 - DNA, 22 bp, synthetic construct
  source 1..22: mol_type = other DNA
taatactgtctggtaaaaccgt

SEQ ID NO: 73 - DNA, 83 bp, synthetic construct
  source 1..83: mol_type = other DNA
cgccggccgatgggcgtcttaccagacatggttagacctggccctctgtctaatactgtctggtaaaaccgtccatccgctgc

SEQ ID NO: 74 - DNA, 23 bp, synthetic construct
  source 1..23: mol_type = other DNA
acttaaacgtggatgtacttgct

SEQ ID NO: 75 - DNA, 23 bp, synthetic construct
  source 1..23: mol_type = other DNA
taagtgcttccatgttttggtga

SEQ ID NO: 76 - DNA, 69 bp, synthetic construct
  source 1..69: mol_type = other DNA
ccaccacttaaacgtggatgtacttgctttgaaactaaagaagtaagtgcttccatgttttggtgatgg

SEQ ID NO: 77 - DNA, 24 bp, synthetic construct
  source 1..24: mol_type = other DNA
tggaagactagtgattttgttgtt

SEQ ID NO: 78 - DNA, 22 bp, synthetic construct
  source 1..22: mol_type = other DNA
caacaaatcacagtctgccata

SEQ ID NO: 79 - DNA, 110 bp, synthetic construct
  source 1..110: mol_type = other DNA
ttggatgttggcctagttctgtgtggaagactagtgattttgttgtttttagataactaaatcgacaacaaatcacagtctgccatatggcacaggccatgcctctacag

SEQ ID NO: 80 - DNA, 22 bp, synthetic construct
  source 1..22: mol_type = other DNA
tagcagcacataatggtttgtg

SEQ ID NO: 81 - DNA, 22 bp, synthetic construct
  source 1..22: mol_type = other DNA
caggccatattgtgctgcctca

SEQ ID NO: 82 - DNA, 22 bp, synthetic construct
  source 1..22: mol_type = other DNA
tagcagcacatcatggtttaca

SEQ ID NO: 83 - DNA, 22 bp, synthetic construct
  source 1..22: mol_type = other DNA
cgaatcattatttgctgctcta

SEQ ID NO: 84 - DNA, 83 bp, synthetic construct
  source 1..83: mol_type = other DNA
ccttggagtaaagtagcagcacataatggtttgtggattttgaaaaggtgcaggccatattgtgctgcctcaaaaatacaagg

SEQ ID NO: 85 - DNA, 98 bp, synthetic construct
  source 1..98: mol_type = other DNA
ttgaggccttaaagtactgtagcagcacatcatggtttacatgctacagtcaagatgcgaatcattatttgctgctctagaaatttaaggaaattcat

SEQ ID NO: 86 - DNA, 22 bp, synthetic construct
  source 1..22: mol_type = other DNA
ggggttcctggggatgggattt

SEQ ID NO: 87 - DNA, 21 bp, synthetic construct
  source 1..21: mol_type = other DNA
atcacattgccagggatttcc

SEQ ID NO: 88 - DNA, 73 bp, synthetic construct
  source 1..73: mol_type = other DNA
ggccggctggggttcctggggatgggatttgcttcctgtcacaaatcacattgccagggatttccaaccgacc

SEQ ID NO: 89 - DNA, 21 bp, synthetic construct
  source 1..21: mol_type = other DNA
aggcggagacttgggcaattg

SEQ ID NO: 90 - DNA, 22 bp, synthetic construct
  source 1..22: mol_type = other DNA
cattgcacttgtctcggtctga

SEQ ID NO: 91 - DNA, 84 bp, synthetic construct
  source 1..84: mol_type = other DNA
ggccagtgttgagaggcggagacttgggcaattgctggacgctgccctgggcattgcacttgtctcggtctgacagtgccggcc

SEQ ID NO: 92 - DNA, 22 bp, synthetic construct
  source 1..22: mol_type = other DNA
actgatttcttttggtgttcag

SEQ ID NO: 93 - DNA, 22 bp, synthetic construct
  source 1..22: mol_type = other DNA
tagcaccatctgaaatcggtta

SEQ ID NO: 94 - DNA, 64 bp, synthetic construct
  source 1..64: mol_type = other DNA
atgactgatttcttttggtgttcagagtcaatataattttctagcaccatctgaaatcggttat

SEQ ID NO: 95 - DNA, 21 bp, synthetic construct
  source 1..21: mol_type = other DNA
ctttttgcggtctgggcttgc

SEQ ID NO: 96 - DNA, 22 bp, synthetic construct
  source 1..22: mol_type = other DNA
aagcccttaccccaaaaagtat

SEQ ID NO: 97 - DNA, 21 bp, synthetic construct
  source 1..21: mol_type = other DNA
ctttttgcggtctgggcttgc

SEQ ID NO: 98 - DNA, 22 bp, synthetic construct
  source 1..22: mol_type = other DNA
aagcccttaccccaaaaagcat

SEQ ID NO: 99 - DNA, 72 bp, synthetic construct
  source 1..72: mol_type = other DNA
ggatctttttgcggtctgggcttgctgttcctctcaacagtagtcaggaagcccttaccccaaaaagtatct

SEQ ID NO: 100 - DNA, 90 bp, synthetic construct
  source 1..90: mol_type = other DNA
tgcccttcgcgaatctttttgcggtctgggcttgctgtacataactcaatagccggaagcccttaccccaaaaagcatttgcggagggcg

SEQ ID NO: 101 - DNA, 23 bp, synthetic construct
  source 1..23: mol_type = other DNA
acgggtattcttgggtggataat

SEQ ID NO: 102 - DNA, 23 bp, synthetic construct
  source 1..23: mol_type = other DNA
ttattgcttaagaatacgcgtag

SEQ ID NO: 103 - DNA, 102 bp, synthetic construct; identical to SEQ ID NO: 41
  source 1..102: mol_type = other DNA

SEQ ID NO: 104 - DNA, 23 bp, synthetic construct
  source 1..23: mol_type = other DNA
agctggtgttgtgaatcaggccg

SEQ ID NO: 105 - DNA, 22 bp, synthetic construct
  source 1..22: mol_type = other DNA
gctacttcacaacaccagggcc

SEQ ID NO: 106 - DNA, 23 bp, synthetic construct
  source 1..23: mol_type = other DNA
agctggtgttgtgaatcaggccg

SEQ ID NO: 107 - DNA, 22 bp, synthetic construct
  source 1..22: mol_type = other DNA
gctatttcacgacaccagggtt

SEQ ID NO: 108 - DNA, 99 bp, synthetic construct
  source 1..99: mol_type = other DNA
ccctggcatggtgtggtggggcagctggtgttgtgaatcaggccgttgccaatcagagaacggctacttcacaacaccagggccacaccacactacagg

SEQ ID NO: 109 - DNA, 84 bp, synthetic construct
  source 1..84: mol_type = other DNA
cgttgctgcagctggtgttgtgaatcaggccgacgagcagcgcatcctcttacccggctatttcacgacaccagggttgcatca

SEQ ID NO: 110 - DNA, 24 bp, synthetic construct
  source 1..24: mol_type = other DNA
ttaatgctaatcgtgataggggtt

SEQ ID NO: 111 - DNA, 22 bp, synthetic construct
  source 1..22: mol_type = other DNA
ctcctacatattagcattaaca

SEQ ID NO: 112 - DNA, 65 bp, synthetic construct
  source 1..65: mol_type = other DNA
ctgttaatgctaatcgtgataggggtttttgcctccaactgactcctacatattagcattaacag

SEQ ID NO: 113 - DNA, 21 bp, synthetic construct
  source 1..21: mol_type = other DNA
tagcagcacagaaatattggc

SEQ ID NO: 114 - DNA, 22 bp, synthetic construct
  source 1..22: mol_type = other DNA
ccaatattggctgtgctgctcc

SEQ ID NO: 115 - DNA, 87 bp, synthetic construct
  source 1..87: mol_type = other DNA
agcttccctggctctagcagcacagaaatattggcacagggaagcgagtctgccaatattggctgtgctgctccaggcagggtggtg

SEQ ID NO: 116 - DNA, 22 bp, synthetic construct
  source 1..22: mol_type = other DNA
tgcctgtctacacttgctgtgc

SEQ ID NO: 117 - DNA, 22 bp, synthetic construct
  source 1..22: mol_type = other DNA
acagcaggcacagacaggcagt

SEQ ID NO: 118 - DNA, 110 bp, synthetic construct
  source 1..110: mol_type = other DNA
ggcctggctggacagagttgtcatgtgtctgcctgtctacacttgctgtgcagaacatccgctcacctgtacagcaggcacagacaggcagtcacatgacaacccagcct

SEQ ID NO: 119 - DNA, 22 bp, synthetic construct
  source 1..22: mol_type = other DNA
ctcagtagccagtgtagatcct

SEQ ID NO: 120 - DNA, 21 bp, synthetic construct
  source 1..21: mol_type = other DNA
agctacatctggctactgggt

SEQ ID NO: 121 - DNA, 110 bp, synthetic construct
  source 1..110: mol_type = other DNA
gctgctggaaggtgtaggtaccctcaatggctcagtagccagtgtagatcctgtctttcgtaatcagcagctacatctggctactgggtctctgatggcatcttctagct

SEQ ID NO: 122 - DNA, 22 bp, synthetic construct
  source 1..22: mol_type = other DNA
cgtgtatttgacaagctgagtt

SEQ ID NO: 123 - DNA, 22 bp, synthetic construct
  source 1..22: mol_type = other DNA
tgtcagtttgtcaaatacccca

SEQ ID NO: 124 - DNA, 110 bp, synthetic construct
  source 1..110: mol_type = other DNA
cctggcctcctgcagtgccacgctccgtgtatttgacaagctgagttggacactccatgtggtagagtgtcagtttgtcaaataccccaagtgcggcacatgcttaccag

SEQ ID NO: 125 - DNA, 22 bp, synthetic construct
  source 1..22: mol_type = other DNA
accgtggctttcgattgttact

SEQ ID NO: 126 - DNA, 22 bp, synthetic construct
  source 1..22: mol_type = other DNA
taacagtctacagccatggtcg

SEQ ID NO: 127 - DNA, 101 bp, synthetic construct
  source 1..101: mol_type = other DNA
ccgcccccgcgtctccagggcaaccgtggctttcgattgttactgtgggaactggaggtaacagtctacagccatggtcgccccgcagcacgcccacgcgc

SEQ ID NO: 128 - DNA, 22 bp, synthetic construct
  source 1..22: mol_type = other DNA
agctggtaaaatggaaccaaat

SEQ ID NO: 129 - DNA, 22 bp, synthetic construct
  source 1..22: mol_type = other DNA
tttggtccccttcaaccagctg

SEQ ID NO: 130 - DNA, 88 bp, synthetic construct
  source 1..88: mol_type = other DNA
acaatgctttgctagagctggtaaaatggaaccaaatcgcctcttcaatggatttggtccccttcaaccagctgtagctatgcattga

SEQ ID NO: 131 - DNA, 1560 bp, synthetic construct
  source 1..1560: mol_type = other DNA
aggcgccaggggcccggttggcgcgaacgccgggttccgagcaccctgggcttccttgtctgcctcccagcgcggcacctcttcggggctcccgaaacctgagctctcgctggttttaggtccagactggggcctctccaccggttcctcccccgccccgggctctggggcccattctttgggctgaccctgtcagggcagagtccgcgcgtctgcctgccattctccgcccgcataaaagcacgttgaaggtgtctcgggcagacacctccaggttttgaatcagtttattccctttcactgttcaaagcagctgttcaaatacacaggctgcttacgttgacgtggagaggatttcaaacaacgctaaaatgctttgaactgacaaggtgtcttgatatctccctcactccatccagcacagctcctcgagatcactcgctaggacaatggctgagcaggcgattcgtgcgggcctcgccacctcggggcgcggactgcggggtgtcctaagccccttccgcaaggacaggatggaggcacctgtaaggagatgctggcgccaccccagcttctcccaggtccggaggaacctctactcagtcaataccctgagctggacttgtctgaagaaacggagccgactccctcttgccggggtgcgctgagtggaggggaaacatcctcgaataacagaactacaccaaaaagacacccatgttatctctcacactttcacactcctcgagatagtgagccggacctgggtcttagtagcacccagtaccttgacacaaacctcccaaatttccacctgagtaacagttatggggtcagtccatgcactgtaacttgaactctaatttattaactatttcatctagtaaacacactcacaccatatataaaatagcatttatttatttctatataccaggagttggcagaaaacccaccgtgaccactcccatacattgagctggaggcacacaattactaaaacagaggtgaaatggtattcatttgatcttaattttttcttatttatgtagtcccaggataatagaaatcaggaaacaaaagaaaacaaagaattttctgaggagatggccattgggggagtggaggtagcagctggtttaaacctaagtaaaactagaaaaagaaactgctgtttcctttttcttatatccaccttagaggatcatgtttgaacgtccctactcctcctcctctttttaaaaagccttgtctcagtcattcattcctgtgcttcctgctcttctgctagaccccagcagctgtttgatttggtgaggcccccctccaacctctgagtggaacttcttttctaagggcctgcagaatgtcaaaactgaggctctggcttcggagctagagctttgaacagccaatccacacaaaaaggcagctggctgctttaatgaaaactgctataaagcttcaagaactttagccttgggggatgcatttataaggaacatggaaaatgcatttccaagttgctggttcttgggagagacataataaacatttacc

SEQ ID NO: 132 - DNA, 2754 bp, synthetic construct
  source 1..2754: mol_type = other DNA
aaaggtaccggaataaaaatttgactaaagtttgggataaattaatcggtggcttgaagaacagtagcacatttctgattatgtaggctccagccaggatttaagttttgtgtcatgattgttttcatatttctttgacatctattgatttggaggcagctgcaagcctttgtcacaggagagaagagcagtcacaatatattagcttgatggcataactcgaaccggagaagcgaggggagcctgagtgactggggcgcacatgacccccctcctggggcctttgggctcaacccttacttttaacttgttctaacaggaataatggagtagtactactaatgcatggtcagaaggggcttttctgtctagaacgcaggcggcgggagttgcaccgccacagcccgggagaccaaagagtctccactccccgctcccggcgcctcctcgccgaccggagccccgagcccagtgagcccagccggtcccacgtcgagaggcgctgggttgggagaagttctgctggtcgtgggctcggcccccaggcgccaggccgagtgcccacctcggcttctttagggggcctacagcgggcagccgagcggctggcggacccggagcttgggaggcgaccgccaggctggtgcccggcctgacccggcgttcgcggccgcccgctcgcccgccgcgggccgggagcgtacaggagtgtgacgcagattgtgaaaacagaagggagggagttgggtcatttccttcgctaatcatcgccccctcctgctcctcctccccacccccatcccatcctcgccgccacccagcctcgaactctcttccttgccagagtctgcctcactttgagccagtggctgcactttatggagaaaatatatgtgcccatcactcacccatctatttgcagcgtccatggagcgcctcggggaacacgcatggacaattaaacacacccacttacggcgggatgatgaggctttctgttaaccacatgtgttcagaaatggatacagccctaattttgacacggaatgcagctaatgcaccttgctatagatgtacgtccgttggaaaagtgtgtgatgggcacctgatctacaccaagagaacagggctgtctgcagaagacccagcggcctgacaccttttcttgttgccggggaaggaaagtttttcttttgatttgtcctctcacgatttttcccttttcctttgcattacagtttcagagaggaatgtaaatattttgactagacctctgcaggggccttttaaacaccaggcacaatcatctgtggaatactgtagttcatactttcaaaacttgggtgttaaagagtctgtcttgctgaggaaacccatagaactagtggcgacctttcaactccctatttgtgtgatgaaatgaagtatgccctagtgattgtcgaattttaaggacttgttctgtcactgctactttccgatcgtttgcagggaagaaaaatcaatcaaacagacctttgatggggttgtggcaaggtcacatttcactgtgctccaggcttacctcttcttaacgcccctccctgctgtggagcgtgtgcaggtagtatgcaaaactctagaaataatatttgaaaaaattaacaccttgtttatctcttctgtgtatttgatataggggtttctttcttttcctgttttggtgtttaatacaaggctgatatattggaacagaatgcagacttcaaagaaactaaaattaggaggcaaatagcgtcaaggtttcctcggtttctgtatgtagtacaaatagtatagcgtcttttcatccaaaaagaagcaaaaattgccaggaggcataaagcatctctgaatatccgttttttttaaagaaacaaacatttcccaaatattgatgtacgcatggttttatagtctgaaaatatttctaggcacaaatatggcatacaaacattcgtttcaactctgtcgttgttgttaaaatgacagtaaattatagtttattttaagatacctccctgttaatgtcagttgaggttattttagggcctggatgctctaggaagaccacctaaaatgtgtttatttctctgcccaattcacctatagtgtaaggagcattttattttattttattttattttattttattttattatactttaagttttagggtacatgtgcacaatgtgcaggtttgttacatgtgtatacttgtgccatgttggtgtgatgcacccattaactcgtcatttagcattaggtatatctcctaatgctatccctcccctccccccatcccacaacagtccccagagtgtcatgtaaggagcattttaaataaaatattttgggcatccttaagtccttttttaaaaatccaacattctacaattgtgacaaataggctgaaaaattttgttttgtttataccaaattcattttaataaagcatttgctgagattttaatacaacaaatgccaacaaaatgcatcagagtagaaaaaagaaaatattttcagaattgcctataatattgcttgtgatcctaaattttttgatgcaaggaacttgaatttttcagctaatattattctgttttcttattctgtgtttctattagaacactcacctatgctctgcttcccttgctgtcttcccatagcttatgtgtgcactatgaatttcaataaatataaattactgcttaaaagaaaaaaaaaaaaaaaaaa

SEQ ID NO: 133 - DNA, 1052 bp, synthetic construct
  source 1..1052: mol_type = other DNA
aaaagtttgccggcttccgcagtgatggatcaccgttttagtggcatttaaatccccggcgctccgccgtctaggtgacgcgcagtcgcccccccaggcagcctaggcggcggcagctgctgcggcgactgcaaaggccgatttggagtgctggagcgaagaagagcaaaagctgcgttctgcgcgcgcccgactccgctgcccgccccgccaggcctccgggaggtgggggctgggaggcgtcccccgctcccgccccctccccaccgttcaatgaaagatgaactggcgagaggacgaatcgcatccaggagctgcgcagccctggccgctgccgggacgccctgctccgcgctgagcttggggccagaaaccagccatagtccccacactccgccgccgcagctgagatttagcggaggaaggggcgagggaaggtagggagcaaacctatgaagaaacatcgcgttgtcattggaacttccaagcctttgctgttaagagccaggttcttaaatcaacccgccccacacacatgttgcttacatgctgcgttttctcacggtctgttttgcctgaacccatcaacagctgggagattaatcaaccacactgaaaatgtggagggatttatgggggagggggttgaaatgtgggtgtttgaaacaaaagtgtataaacaaatgaattgttgataacttagttattgacctggagactggtagcttattaaagaaactccgtgttactcattcctggagttgggggtttctgtaggcactttatttctccactttcaagagcttgggcttggcccaaatcttagactgtccaattctgcctctattaccaatttaaatctatggcttgaacctgtgcactgaaaatcaaatcctttaaaaagaaagaggagaagaagaagcaaaaaagaaagaaaaaacacttattagaagccctagtcattttttggctttctgttttgttgctgtccattgaagactttgaacatgccgccttaataaatgtattaaaattgaaaaaagaaaaaaaaa

SEQ ID NO: 134 - DNA, 5206 bp, synthetic construct
  source 1..5206: mol_type = other DNA
gagcgacgcagccgggacggtagctgcggtgcggaccggaggagccatcttgtctcgtcgccggggagtcaggcccctaaatcgaagaagccctggcgcgccctccccccctcccgggtctgatagcagactccttgaaagcagggtccttgtttagtgcatctttgcccacatacaccacaacatatcaagatgcatttattaggaaggaggagtttagagagcaggctatcagaataaccactcatcctgtgcctcctgattgctgagtgttcacctggaccttctgactaccttccctgtgctattccatcagcctacagacctggtacctggatttttgcccgagatgattcctaccaccttactactgacgaagacacccattccagtggaccactgtgacccaggaggcattcagccatcatgatgtggcctttacctccactcctgtcttgttctacccagattcagcacagccctttatagtgaagtcagagtcctcaagccaaatagctaaagctgttttatcacaacaaaggcctagtttgttccatgagtgtgcatttcatttcttcagttaaagccttcagagacacacaataaatttggaccaggggattttttagttattaatgctctctgaagaaaggcaacatctttttgagagcagcattggaccacaccccacaatctcaaatgattgaaattcatgaacatctaggatcccgtgaaggtcactggaccctgttttttctacttcaaatcctgtagtagcctactgaatgagaaaacatattctgacccattgggatcaaatcaaaggcacagtgaactcctcatagcatcttctttggaattactcaggaaccagaactttttacacaaatgtaagaaattctaccaaggagtccccttacctaacagcatctcacaaggctgcaccagattccagaaaaggcttctcttgatacatcaagcattttgtgaccgacttattcttagatcattggttttccaaaggctttgtggccatgaagccctttgagtgaaaactgtgcagaagcccagagtaaaagtgaagctgctctggatgaagtagtgaagcaagagtaggggcctgaatcctgctacaactatcttcctttaccaccgtggtgacacctaaggggacttccttacaacaccttgaactcttccgaacacagtttgaaaaccactgccccagacagcaatatgtttgacctgaatggcattccaatcttttctgtacctccactcagcacagttcatgttcagtagatgctgaacattcttagaaatactgtgtgtgaacttagaaaagtgcaagaagacaggcatgtctttgaccccaggaatgatcatttgctgaagatggtgtcaagtgaacctagattaacagccctccactccagatggatatccagtgattcctagaatgggatatagccagagaacaattctatgcaccctacactgacagactcccttaagcaacaccagatgctctactggtacttgaagtacatgactttgaagtcttgaccctccatgaatacctgaattatcagcaagcgggttttgaagctggtgcctcattgaggccatattagagcaacttgtacatttgacctcttgttatcagccatggtactctacttcgtgtgcaagagataactatgaaagccaaattcaaatactggcaacatttcctaaaggggctcaatatctatcattcgtcttcttttccaaactacacatcactgtatgactcaaccagtagcagttatattgccccttggtttttattcagtttaactactgtttccaagataaatgagctaataagctttaaaaaaaaaaaaaaaaaaggctgaattcttttttcttcatcactggcatatctgcctattctccagaattattatgactattcagctcactttaacagttgaacttcaagcgacaatctttgaacaccccttctcatgtgatttaaaatgaaaccatttggaaaagtttcttctagccagtaatagattttttttttaattgctctgccttgtgccgagagatgttcttttaagatgaatcttttgatgtctgataccaccaaatataggtggtagggagagttggaggctggccctttgagcaggccattagcttacttgctgggcatttccgatagcttattgcctacctttttgctggaaacaaactgatttgaaaaacaaaatctatgaagactgcagctaaggattttatcggtagacttaagagcttttgtccttgtggatattttagtggaaccacatcagtctcaatactgtcattttacactgactcagagcagctgacttcattccttgccatgatatatatttaaggcaggcattgtaacagacataaagacaacttatctgtttcagcaggaaggattcagtttatgaactctcagaccagatcatgttgaacaaggagactttgatgtgtgtcatgagaaaactcattctttacttcccagtcaatttaaaggccagctatcctgagctactcgaatgaatgcactggttaaacattggaaatagtttgtttatatccttgtctctctctaggccaattgtgattacatgactcgactctacatctcgtcaaacaaggcctaggtctggttgctgtagactgctcgccctcaacaaataaaatctggttgactagcctccttgtatatacaactattatttgttaagaagaaattatcgtcaattttctactaccttccaattgtcagctctttttttcctctctggtttttcctatactttacagaaaaagacattgatctatactgccattccctctaatcctgccatactcagtcaaaaggaatgacttaagatgaagatgatcatctgctcgagtctaaaatatacattgtatataagaattggtgattagaaaagcaaaaaacctaaaacttaaatctaggagtctgtatactgtctccatgtctccatgcctcagatctcatctaaatctttgaacagcaccattcaaccaatctgaggccttgacttgcttgtaagatgattctcagagatcggctgagttaaaaaagatgacgacttgattaccaaagaaagtagggccaactttgacaaatctggctctgctgaccctgtcactcccagatgtagcatagactcctaaacagaacctcaagtctgattgaggataaggccttctcctgagctgaaagttctttggcagatgagcaagaaactgaaagctgatgtacctgactggctctgtaagatcagaaaactgtatccagaataagccctatggattaacccctgagtacccagagtaaaaactaatttacagaacttccttattgatctgctggttcttccagatcatattctggctattggtatggctggcctttctgaaggtaccctgcttgtctattttcctgactcagctcttgcctgcctttttcacatgttgctgcaattagactcaccgtgaggactacagtcaatttcagtctatcttgtgcccaatacaacaaggatttttaatagtaacaacccacacctcacccactaggactcaatgttcacaacaggaaggaccattgctgcatactccttgaccagcaacttttttgaagatatttttaagtgcagagtaggcctctattcctgtatgtaattgttcattttcagcacctggaacctcatctatcgggtctggaaggaatacagcagttcgaaagccgcgtccatttctctccttcagtagtgcagaaatgagtccgattcaccagtacacacagaactgtaccagttcaacctagcaaaagaagaaaagtttccactgtacttaaaatttacagctgactcaaattgcctcacagaattatttgatgtagaaggctagttgtcttacttcagatcagcaggacagttgggctctcagactcatgaccactgagtttgcttgtgttgaaactgtggtttcatccaacatatgctattggacatgattattattccattcaaatggattacagacttcttgaggacaggacaaacttatctctcatggtgtttttttagaatacttttataaccaaggaagaaaccatgccagctgttaccattcaacttcttaagcagagattaagctttttcatatctgttcttatcctggacatcagtagtttttaattgcccagcatccgttccatcttgtaacaactccctgatgtttcttaaaaccacctcttcctattttcagtctgtggtttggacagtctgacccaaccttgagctttgtgggtgaacatgtaattcagacctcatcaatcagcaaatccatctgaactgtggaggagaagctctctttactgagggtgctttagctttgtaggatgaaaacctcaaactaacagggcctaccatgtagagaatgaagccagtgcaggggaaagcagagccaaaatatggagagacttgaatcctgatgacagcgtttgtgcccctggatccaaccgtgcctgaagctagaatatcccctggacttttcagttatgtgaaccaataaatacccttttttgcttaagttactttgagttgggtttctgttacttgaaattgaatccacactaatatatctaccaacattgagacttgacagatccaagtatttattaagctagaggtcatggtcactgaaattactttccaaagtggaagacaaaatgaaacaggaactgagggaatatttaagatcccacagaagcgtaaaaatgacatggtagaaagtaatagaaaacctaaatgtctgtcattaaaggataggttaaggtgtggttcagccatataggaatatctcgtatctgttaaaatgaataaagtacattcattgtgtatggaaaaatggccatgatacattaggtgaaacaagttattaatagaaaagtgtacagtgtgaactcattttaaaatgtgtgtgcttatgtttataaatgcatagaaaggtctattcacagctttctttgaacagtgtagatcacatgaaactttcaactttatacatttctgtattaatattttacactacccacattatttttaaactttattttaaataaagaatttttaaaattaaa

SEQ ID NO: 135 - DNA, 2100 bp, synthetic construct
  source 1..2100: mol_type = other DNA
atttcatttgtgattcggatgatgcagctctaggtggattgactatgaaaggcgctgaatatcttcaggaaaatgggttccatgagaagtgacatcctcctcgaatcaaaagattctcaggatgatggagtgagtgatggaataggttgccaactgtagttactccactcagcacagaatggctgcggggaaatataatcagacatgcccaaagaagtctgcttagggtgaaacaaatacctttgaggtttaaatcaggaaggagagactgtttctacaggtgtcatacaaaaatcaatactgagccaaggattattggatcccatagaaccacggagacttggtagtgaaattcttccaaagcattccatccgtgcaggcagatggtgttcctaggtttggaaatataaatcccatgggaagtcagctgaagaccattttatgcatcactcatcctgttagagccctgtgctgttctgtctctctataagactggctgaaaccaccacggttgtttacaatttatgctctaactttggatcacctagttaatactctaactccgattattaccaaagacaatgttctataaaagatattgtctattagaaggatgcttctgcattattaataaaatcacagggaaaatgcaacattttggatgtcacatctgataagttggcagttaaagaacagaaacagcaataagactgcgtggctacattttcccagtgccaccaactgggatttaagctgactaacattttgaaagcccagcccatttcattcacagcaggatggcagtgggtgactgatcgtacccgccagctggctaatgtcataattaacaaagttgctactacatctgcaaagtttaatcgaaaaatttgccagtgatatgatggccttgtcacagagatcacaagggacacggagttgacttttgctcaaggtggaacgacagagctgtgtaggcaagatgagaaattgcgaacttgttcggccaaaggattatctcctgagatgtgtagaaatgaactcttgtcagagtttcaaaggaattaacgttttttggtaaaatgtgctaatgtaaagagtgagaagattagtgatacataatgaaagcaatgcattctttcacatggaaatgggaatataataaatgtcctgcaggtgattggggctgtagcaattttattgaagattaaagtttgattgacctttgttagggtgattaacccttctgaaattaaaagccagagctgaattattctgctcaatttaaatgcaaacatcgagcaggtcgtatgtcattcaaataataataagaagaaaatcttcctattttaattttcatttttgcacttctgagtggtatgctgctttagaaggaccatagctctcgcaaaagcattctggtggcagagaatatacaataacgttacagataattgggagcatataggaagagtcccaagcactactgcctgcattcgaaacatcaaaaccaccatcccaacaaaagcacggagcatgaagccaatctctgtacactgaaaatgcatgctcccaatagaaaaacaacctagggatgtcctgaaggggttaggaataaataacaatgacatctctattctagttcagtgccaaccaccgaaattgcatatcatatccatgttaaatctacatgatgtctcctggaaatgcgtgctttttggtacagttgcttatgaagcagtttctaaaagttgattatttctgatgtctggaatttcattctccatgatctctggcagtttctgaatatattcagtattttcattaagttcaaaaatgctgtaggagaagctaacgaattatttgcaatgccatttaatggtttagttttatttttctctgttaaattttctaagccttggtgaaaagttgtgtattcacatttcttcaaaaaactgtttccatttttcatatcatgttaattttgtttagttctaagtcagtgaatatttttagtcttaggtcccaccacatagtgaatgtgtctgccttttatgtatctgtgaactattaaaatgataataaaggttctcttccttccat

SEQ ID NO: 136 - DNA, 2687 bp, synthetic construct
  source 1..2687: mol_type = other DNA
aggtacctatattctgcacgggcttctgggcaggcagcactaacactcaggagaaggtctgcctctgtctgcgccttcctggttcaatgcaggcagatgagtgtcacagatgtaactgaggcactgaataatcaacatatcggagccaattcccaggacggcaagagggggaaccggcagagatgactgaccctggaggacaagagcattcttctgacaagttttagcatggactatcctcaggaaaatgagatccaggaagagtgacgccctcctcgaactgaaagatactcaggttgatggagtcagggacgaagtggcttatcaacaacacaggatgtctgcagggaacagaacttattcagacatggcccagattgtctgcccgtggggactgagggacctctgagggttacatcaggaaggaaggagaggctgtttctacaggtgccacagaaaaatcgatgctgagcctcggattattggatcccatagaaccgtggagacttggcagtgaaattcttccaaaggcaggcagaggttttcctggtttgggaagtataaatcccgtgggaagtcaactggaggacattttacgcgtcactcatcctattatgagcctgctgctcctccgtctcttgacaaggccaccaactagaagggcacagaattgttcataaaacagctgaatgtgaatgtctttggattcccatttaatgagggtgtcttaatgagatgactgaagagtacaaccgccaaggctgagatgatgcaaaggctacgtgtgggtacagttcccggtgcctgtgcacaccattcattggcagctaaaggacaggagcagcaataaggcttcctggctacattttcatggtgccaccaactgcgagttaagctgacaaacattttgagagctcggcccatttcattcccagcaggatggcaggagttaactaaaacagaataaactaaaaattgtgccttcattgcacatttgcactgattatcagcactgaaaatggctaagttctgcttatcaagagcgggtgactgattgtccctgccagctggctaatatcataattagcaaagttgctaccacatctgcaaagtttaatcgaaaaatttaccagtggtatgatggcattgtcaccgcgatcctgagggaatcagagtcgacttctgcttcaactggaatggcaaggctgtagagctgagatgagaaactgtaaacttgctcagccaaaggttatgcttcacatcacccaccagggaggatcatctctcatgaaaactttcaaatttgattttgaaaatttattcagaaaaatttaagccacgctctttcccaacacaggaaataatatgctccagagaatcctctaattcaccgaaaatggacttggataacatttgggttctcatactaccacagtcctcttgagaaggacagccaggcttgggtggcagtggtgaggaaggttcctttctgtcaagggagctgatgccttaaagaagctatctccagataaagggagatctaaggaaggaagccaacgaaagacaccatgtaccagaaagccaagcaggattttgacatttataaccataatctctataaacaagatgaattatggctggggattcagttagtaaatcaacgaaaataaatcctggtaaggtaatgatacaattttatcattttttgttgaaatctaacagtgtatagtatgctttgctgcaatcaaagacctgcagtattttgtgtgctaaactccaatgacccatcatttattttctctttattccacctacttacctgtgataataaccaaatagcctcacatttcccctttcaaagttgtatctcaagaggaaagctcactgaccccacacattagccctccagtagctccagctgagaagtactctctcctccatttttttcagtgcataattcaaagataacccaaataagaacagaaagttgtgtgtcatagtcactggcatctatccggtgcttctgaaccatgaaccacatgacctgattaaagaggttgctgtgagtgtgttgtactcttcgtgaactaaattccatgatgacagaaaacaaacctgaaaacaagagttatttcacaactctagagctccacagagtcggctgcaattggttgagttgaggtgaattttcagtggacctggatcagtgtagaagggcaggaacattggcccagcacagccccaggctgtgcagcattcaatgtgacttgggactggccttcctggctctgccttgccggtgaaacatggattggaagctttgaatgttggaagaaataaacaagtcttggttggagtcccagtttcaataaccacatattttcaaacctagacaaatattctggtctcatctcctcatttggattacttcaatcctcggagagaacggaataggactctgtatttctttggatgttcaacacagtgtttaactaatgggtgctatttgaaatattatcgtctattatcattgctattctaataaggatgatacttgctataaatattattgagaaaaaaaacttggaagtgttcttgagcaacataaacagtacaaaatgacagatattaaacaccgttcttttttgcacgtaaaactatacaaataaaaatatagaaaagcttgc

SEQ ID NO: 137 - DNA, 3347 bp, synthetic construct
  source 1..3347: mol_type = other DNA
gtgctgctggcgcccggcccccgcggggtgcagctctgcgcgttctcatgctgtctctctctctttccctccgcgctgcctctccgaggtcctcccgccgagccccggcgcggggcatgaggagcccccgggtgccgcccagagaccagcaggctgcgcgcacacctagccagcggcagacggggacatgagcagcgcgcacggggtcccgcgcccggcggccagccctatccggcggcggccagcgggtcaacgctgcccgggagaatgaggcaggagccggcggcagcctcctttttttccttctcctcgccttcctgcggctccggcgctccgggtccgggccgggctgcggctctgctgcgtgccccgcgcgcccctcaaccgcctccggatgcgcttctcgatctcagcccgctgtcgccttctctctgaccctggtcccttccactgcaggctgttgctccggtgtctctgggctgctccaccacgtggtgcctgcccatcatggcatccaccaccaggtggcctttttcttggttagcctggcaaggaagataaagacatttgcaaccaagatggtaatcactagtgaaaatgatgaagacagaggaggtcaagaaaaagaaagtaaagaggagagtgtcttggcaatgctggggattatcgggaccattctgaacctgattgtgatcatatttgtctacatatacaccaccctgtgaatggcccagagcgtcctcagaggcctcagaatggccaaagacggaagtcctgcgtgtcggcgcatcactgaccagaccctgcgagaacaagcaggcttgacccgcacataccacccaatcaaatgcaccttcaaactttacaaaaggtcacacaaatagaccgatcctgctgcagggagcagacactaaagcacaatgattccaacaaaactcattcacagcactaggaactcaacgtctttggcagggggcccagaagaatgcttggaagaccagcctctgacaccatcagtgagcggatgggtgcagaaattcattattccagatcgctgacagatatcacatatttgaaaagatgaatagggcggacatggctcagatgtgtgtctcccaggacaagtgtttcatcttcacttgacgagctatttagtggaaaaaccacaggcgcagccctttgacaggcatcccattcatcaaaagtgtctaactatttgatactggggagataacttatttttcttttttcattggcttgacatgtgtatctgttcatgtcaaggtttataaatatatatttttaataaatgtgctctattttttagcatgaaccaaatacttggagaggcactcccagatccatagagctttccttagttttatctgctttgtcccctcctcccccaactacagatgttctgttgtggagccattctagtccttttgtctcatcttgagtcttttaccttgcgcttttgttctctctctctcctctctctctgcctctttggtctgaaggacattttcccatactgtcagccatggttttgggtgcatgttttaagattgtccattgagtggctttttgttgttatctcggagatataaaatgattgtgggcatgcagaccttagatgcaccctatctttactgagaattatgcatgaataagggctgagtgatagatcagcttaaaattaaaaggactacctttgaggaagaagagcgtggctatatttgcagatgaacttttgaacagaatattcagcttcttaccggcagcgttattgtttcattcttgtgaccattcgtttatcagattttgattttagcggtcatgtaccgcgagagttgggaagaacaagggggaaagctcgggattaggtgcattactccttcctttgcaagatacctgggatcctcctcaaaagcgggtggggtataaatgacacaagaactcccccaggagatctcatggtgattcaggctgtgaggacagccctgtgacaggtgacttttcagggacatgaggaggggatttaatgattgccctaaaggacttctgtatttttaaagcccctggtttacacccacatgaagctatttcctctctggcagggatggttgcataaaaacaaattagctcccttctggctccctgaaatgggcccttgcctggctacagtggcatggccttaaagagagggttagtattccttctgccattgccagctgtattagtctgttttcacactgctgaaaaagacatccccaagactgggcaatttacaaagaaagaggtttattggacttacagttccacgtggctgaggaggcctcacaattacggtggaaggtgaaaggcacgtctcacatggtggcagacaagagaagtgaacatgtgcaaggagactcccatttttaaaacagatctcgtgagactttttcactatcatgcaaacagcatgggaaacctgcccccgtgattcagttacctcccaccgggtccctcccacaacacatgggtattcaagatgagatttgggtggggttacagccaaactctatcaccagccttgcccctgggcagaagcagcagcagtctgcctggctggattcaaatgattctgaggcttctatagtctatgcctgcagatctctccctcacccatgctatagtgtctgaaattccaccattagagagtcatttcttgggctctgttaaatggaccaggctcttttataaagaaaatgcccctgagcagctggctctggcattgatttatgatatcttctcttccctgccagaaggaaggaagctaaggtgcatgtagggcgtactgtgtgcccaggcactgtgccagatgctttggatacttggagtcattgaattcttgtagtaaccctgtgagagagggagtcttttctccacattgtagaagaaggaaaaagggctcagagaggtcaagaaatgtccctgagatcacatggcttctagtggagtcaagatccaaacccaatgtgtctgattccttagcccttgggggtccggaggctgctgaacaagaaaggaggtggagaggagagaaagctgcaggcataccaccgcacacccttctccctcccctgtaaaaacaaccctgggaactccctggacactagcagaatatcatacactaaggataagggatgagaggaggctggttagaaataaagcagtgtcagggggaaggagctactcagtaggctctgtgtgattctagaaagactgtatgaaaattctgaacagtgaacagaataaacaataaaggtgcaatggaaaaaaataaaaaaaaaaaaaaaaaa

SEQ ID NO: 138 - DNA, 3302 bp, synthetic construct
  source 1..3302: mol_type = other DNA
tcattcccctccttatccctcctcttctctccccttccctccccgcccttcccctctgccgcagcccagctcctagcgcgtcagcgtgggcttcccggagcgagtcgctaggtaacagggctagctccgcagacccgggccagggaagcccgcgcgcgtcgtcagcatctgcgccgaggggcggggaacgcactgacactcccctatcggagcctgcagacccttcagctggttctcagtcccctccctggatgccttaagggtggctattgccaccgcggagttgcccctcccctgcggggaccccgacgtcgccgcccccttctcctcccacgctggtccgcaccactctccgcaccgccagccaccctccgagatcttcccgccgagcccaggcgtggggcatgaggagcccctgggtgtcgcctgcagacaacccagcctcgcgcacacctcgccagtcgctgacagggacatgaacagcatgattgggttccggtgtccggcggccagcctcagccggaggcggccagtgggtctgcgctgtttgggagaatgaggcaggaacccacggcaacctctctctcctccttctcctcgccttcctctggctcccgctccccggacccgggccgggctgtggctctgctgcgtgccctgcgcgccccccacctgcctccggatgctcttctcggttagcctggagaggaagataaagacatttgcaaccaagatggtaatcacgagtggaaacgatgaagaccggggaggccaagagaaagagagcaaagaggagagcgtgttggccatgctgggcatcatcgggaccatcctgaacctgatcgttatcatctttgtatacatatataccactctatgaaaggccccattcagacctcggtggcctcaggatggccacgagacaagggtcctgtgcatccgtctatcagcgaccacacactgcggggggaccagcaggtttgaagaccacacaatgctccatcaatgcaccttcgaactttctgacgggtcacctgatggacccgtccttctgtgaggagcagacactctgaagcacaatgaatccagtgggactccttcaggccacaaggaactcccacaggggacccagcgggatgctggaaaggccagccttctgcaccggcggtgagcacgagggtgaggaagtgcttcaccctggagtgctgaccacatctcacactggaaagaggatgcgtggggtagtcgcagctccgctgacctcctcccaggactggcaagataaataagctctaggtgcggttcgttgacaggcattgcattcatcaaatgtttccagctgtttgatatgggagataacttatttttcctttttcattggcttgatatgtgtatctgttcatatcgaggtttataaatctatatttttaataaatgtgctctattttttagcatgaaccaaatatttggagaagcacttctggacccatagctcttcttggttttaatcgcttttgctccatcagctgcaagacgtctttgtctgtagtcatccggttgcctcagctcatctcaagccttgtcgtacctcaggttcttcttccttcccatccctgcccctcccttctctcctctctctttcccctgtctctcccttccatctcttaattcccctatccttccccatctccccaccccccatctcctctctcccctcctcctcacctcttgtctagaggacattttcccataataccagctgtggtcttggctggtgtgtgcttcaagattgtcgagcggcttttcattgttctgtcagagatataaaccgattgtgggcatgcagaccttagatgcaccctgtctttattgagaattatgcatgaacaagggctgagtggcaggccagcatctccagtttttaaagggactaccaaggaggaagatgagcagccctgcctgcaggccacccttgggacagagcagtcaccagctcaagtgcagtattgcttatttgctcctgtaaccatccaattatcagattctgatttatttatttatttatttatttatttatttatttatttatttatttttgtgaccatgagagatggaaacggggcgagctcgatgttcagtgcaccagccgtcatcccttccttggtgagctgcctggggaggtggagggtgggatctcttcagaagtcagtgggatatgagggaagtgagacctccccgctgtgagaatgcacacacagttcctgctgtaaaaacactgctgggacgagtgacccttcaagggcattgggaggggacttaatggtctctgttaaggagccctgcacattttaaagctgtttttccactcacacaaagctctttttttctctgaggggatggttgaataaatacaaatttgccctgctctctggctccgtaacacagccccaacctggatgcagtgtacagctttaaggagagggtccgcattcattctgccaagggcagaagtggcagcagccccccctttggattcagatgacactaagggggggcatgtctcagcttgcccccttccttaaatggcaccatgaaggagtcatttcttgggcttcactaagggaccagtctcttttataaatcaaatacccatgagcacctggctctgcccagagatgtctcctcttccccaccagaaggacagataccgtgtacacaagtaaccaggctttcatagttgctggtgagagggagagtctttctctctcacctgtgtggtagaaggaagaagaaaggacagctcggaccaggaatcttccctgaggtttcctggcacccagtggagtcagattctagcctcacctggccagttccttatcccagcgaggacttggatgctgttgtgggatgctgatgaggatagtgactgtggctggaaagcctgtcgctttgctgaggatcaacccagcacacacacacacacacacacacacacacacacaccacaccacaccacaccacaacaggaaacaaccttagaactccttggacactggcagaatttgacacaaataaaagacagtagagaggctggttaggaaggtgggaggcaccccggggcgagcactacctaccaggctgtgtgattcttgacaaactgtaattctgcacagtggactaaacaaacaataaaggtgcagtgggggaaactattggtgtcgccaacttcctcgtgagcccgtgtgtaattgtttgatttaataaaacttgaccgtggctgtaaaaaaaaaaaaaaaaaaaaa

SEQ ID NO: 139 - DNA, 3246 bp, synthetic construct
  source 1..3246: mol_type = other DNA
gcgagtgcgtccccaccagcctggccgccgcgggaactcggggaccgggagggcgggggttagcgggaggaaagggtaagctgaagaggggctgggaacgggagggaggctcggggcgaacgggccgtcgcgttcatttccataagaaagctgaaatcgcagttggaagcaggatttctcgcccctgcagcccttcccccatacccggttttctgcaagaagcgggctccagagctcagagcccgaagtgtgtgctctggtctttgatgatgttgataaaatgtggtttcaaatgttgtcccgaagaggccggtttcctgtctgtatgtggaatggaaggaccgtgccggagttgtgttctcaccacccactttgtcggcggaggagaaacactgctgcctgatgcttgcactgctgcactgttcttgcacctgctcctagccctgaactcgggccctgagctcctcactgccgcccgctctgcgctgagccccactcctgagaccgcgaggtgccagagcagcgcctctcccaccaactcctagacgcctgtaggagctgcgccggagccttcagaaccccatgggggtgtgggggtgaagttggaggtcatccgggtgatttttttcttactttttcctaattgttctcctgcgttgatttcaactcttccagtctcccggtgctggggcgccaatcctttcccaacaattcctacccaccagctggaggtcggggcccagggttaccccagagttctatggtggcagggggcgccgagtagaggggcgggaggagctgggtcctcgggaaccaaaggggacctcagcgggggcggagtgcgcagccttggctgctgggaaatgcccaactagccgctgtgagtgcccacagcgcgtcgtgggggcgtccgttctgcagctcgggtgactcagacgccctgccaaccccaccagctcccggcttctcgaacgctcttctgatcgcccagcggggagacttcagggcctgcctcgtcaggccagattgcagcccgcgaccttcccggattaaccaagggcggggcctgttgcccagcaaggctgctgcctaactgagcctcccggggaacagccatgaccaaagccccgcgggcccaggcgcgggcacctacccctacccgctcaggcctgtgggagcgcaggggcagaggccgagacgctggacttccagccccacacctctgacaacccgggcacgtctgcggagcccgggggaacagtcagcgggctctgcgtcccgggagctggggacacgcctgcaaggtgtgtgatcctttaaagatccttagtactagttttgtagggagcctttgttatagctttgtcagcctgcgcgccttcagcagctccccctcccgggcccctcggttccctccgggctcgcccccagcggagagggcccggcgcaggggatctgcccaggatcaactgccctcttccagatccccttctgcgagattcttttcctttggttacaatctcccctccgtgcctggggcagataaacacggttcgccccgcctgcagtattggggcgatgctgtcctggaagaacgggctttctgaatgtgacctcgggatgctgcagcggcgcaatgctgctactgctgaatgagtagctctcctgccaccgcagggcgactgcggtgggcacagcagggcgcacagaggagggggtaatctgcccacccaggctaaggatttgagggttctgacaaaaaagagctggttgaggatgacacagttgtgaactaacctggctcctactaccccacagggcagccaggtcccagtagtcttgggtaccactgcccccaccataccaccattgtaaagtatggactcctgcttagccaacgtgcagctctaggcaggatctctgaaggatgctctggcacacagatggtgcaggtatgactttacctgcacacaagcttacatgtgccctgtcatatatggctacacgtttccccatttccccacagtcttcctatttaactggtcttcaatcctcctttctagggctttccgcctgcacacagtcatttggagaaaatcaagtgagtgcacttctccccacagaggattctgtatgttatgtaacacactcacactgggattcctcaagttaccattttgccccaactcagccagcacttggttttctggcgggggtgtcaggatgatgcaaatggaggtggaagcttcattccctgggggcttttattcccctccttgaagccccagttcaaggcccaggaacaaaggggaaaggtaactgatctctcttttggtctttctctcccctcacttccaccccttcacctgcaggccctcctctttgataaatagaggaaagcccgtgggatcaggtacatatgtaaatactgctgacctcggccgcccctcctcaccgaaaagcagaaagcccgtttcaggtgcaggctgctccgtgcctttgagtgtccaccacttctattcccccagactgcttgggactcaaggccactcacttaatacccgtctcttgttaaaaacattcaggacaactattaaatagttattggaagagttaaagaccttctttcaggagtgaaacgtttttaaagctgtgcttggcataagtctttaaggcgtacaaagtagaatcagtagacttgcctgagcgattttgaaatttgaaagatttctccccatgaaatggtaatttaaaatagtttgtagatgttgttttgttctgtgatggctttttcaccattgtcacctaaagattctcaagaccaagctactaagacatttcatttggttagtttctgggtgatactgtgctttctgattagacctatttaatttatttttaactctgcaggagagttttcaaccttggaaatgtaagtgaaacacagactcaccctccttctgagaccttgataggggcagaaaggaacaaatctggctagcccccctcacacctggaagtttccctgtctaaacctgggtacaatgagcgtggtgggttttgtcaccttttattttattgctggctcaaggaaaattctttgaaatgaacatctattctatttcagtgtgcaatgttccagccgacttaatgggcttgccttatgcggagaggcagtcctgcaggacagagctaatgtgtggtctcccaggaagctcatcagaacacctcttgtcccttttcttt

SEQ ID NO: 140 - DNA, 463 bp, synthetic construct
  source 1..463: mol_type = other DNA
ctaggcgactggtgtggattggtgttgatgagatgtggcttccagcgttgcccgactcagacctgtttccagtctggatggagcggaccgactgtgtgggatggggtgagaggaacaagtgaaactcaacagctaaggagaaaatgaagaactagtgctacgttttccacgttctttttgctccggggttgacatggcatttggcctttctacctggagaaaatcaagagtatgtaactttccctgcagatggtctgtctgtaagagctgacacaggaacctttgggagccagtatctattttgtttccgactgcaaagttccagccaatgatttggaggggggccggcaccttctgctgggagctagtctgggagggaacaactcattcctgagttctctttcgcaaacatatctcttgtctattaaaataatagtaataaaaataagaataataattaaaa

SEQ ID NO: 141 - DNA, 2190 bp, synthetic construct
  source 1..2190: mol_type = other DNA
tttgttttttaaatcaagattatagattttggggaggtgactcaggagttaagagcatttgcaactctccaaggaggcttggattcagttcctagtacccacatggtggctcacaatcatctataacttgagtttcaggaaatccaatactctcttctgacctctacataaaccaggcatgcacatgatgcacataaatattgtgcaggcaaacaccgtcccccagacactcaaaatacataaaataaaaataagtattttttaaaagttggaatttggtatgattagctttatagctttagctatttatattagttttatggcaacttgacactacccagagtcattttggaagagggaacctcagttgagaaaaatcctccaccatattgatctatgagcaagccagtaagcagcactccttcatggcctctgcatcagctcctgcttccaggttcctgccctggttgagttcctgccttgactttccctcagtgatagagtgttcttgggagtgtagatgaagtaaaccctcgcctcctaaactgcccttggtcatgatcttttatcatagcaatggaacccctacttaagacaattatctttgaagcacatattttgacatttaggcatatcaataaatgattgatggttagtttcttaaaatgccccttgaccacaaagacagtgaattttctcccttgctccctagcatagattataggtctgagccatcagatctggcttgaggttcaaagcttgtgaccacctgttccaaggacaaatgagctgcctagaactcttccactggcctgcctgtagccgggaatggtggagttccctgtgactgtgggattctatatgagctactgatgagacataagcaagtaaggaaagttgatcttgggatcaacactcatcaagaataagatgggtcccccactctctagctgtgcttccagacaggtttgcttcagtgtggcttcagcaaagccttcctagtggtctacagtagagaatgcaagagtccatccctagcctgaacactgcacacagacaccaattatgctaaaatatttaatttcattccataaataattaagcctaaataataatacctagatataacttggaacttgtgacaatctacctgcctctgtctctgtgtaccaggattataggcctgggcacacattcttcttcaggactgattataatacattttgtatattgatttaaaaaaatgatgcttaggtagttatggcctgtgagtaattctataaagtagactaagtggatatcatcagtctatactctaagacattcctttaatttttttaaatttttttttaaagttctactttttaaagattcctagagagccagatgggtgatgaaaagccaggtttcacatgtaacactaatattgggggaaaaaaaacctgacgctagaaattatttagttattctgaaagctgtcacttcttgaaagcagagtcagaattagaaaagaattaacagtgtatcgttgtgttctcacatttccaatgtggctggtgaatcatgaagcaaaaatttcctcaaaactggagccccagagtcttagtctgagcccatcctcctgcctgagaatctccaaattgcctagtgaaggaaaagctatgtccatggtcccacctgttccaaagtccctttggaaatctaggcagaagtaggtttgctagagtcttacttatgacaacctgggaaacaaagaaggctcttcatacaagggtgtgtatccacacaagaggcagcatcctgaaggaaaccttcaggagcctccgatcctgcagttagctgactgtttcctgccctccttggtccaactcgttatggccccaggtgttccttcttgctgctgaactgctgtctctaccagctgagcccactcctgtggtctgcactcactgccctggctgcaggttgaacagggtcaggctacttctcagatgcttcccaccagccgcagtgaactccattttgacatgaagccatttaaatggaaatccagttttacaaagctcaagtgactggtaatgaagatgtgaatgatgcttatgaggtttttatttaattttaaatcaattgtgtattctaattcattaataaaggggaaaggaaagaatacaggaaaaaaaaaaaaaaaaa

SEQ ID NO: 142 - DNA, 10194 bp, synthetic construct
  source 1..10194: mol_type = other DNA
cagctacaaagacgacgccggctgcgctcgcgggaccagagggagggaggctgcggacgagtgcggggaggagtttgagatgggaggcgcttgcaggggctcccccagttctgcgctgtgagccggggcacaaagagccctctgcactagcgccgcaggaccgcggacccgagttggaggcatctgtccacccatgtggttccagacacgttcatgtggccaccatgaccccgtcggcatcacaggggtaaccaaggtccacagaacgaggggaaagggtcactgtggatcaagccaggctcctctgagcagtccagggtctatttacagagagcagcaagttcctgggacagaggagacagaagggaatttccaggttcttcaggacgttcacaaccacactgagaagcatctttgcagataagtatttaaacttaccagcccatagcctaccagcctgccctcttctggtctgtgcaggaaagtgtagtatcctagaatgccaaagtgggaggggaaatgggtgatgtagctcattctcttttctacctctatggaagaaagaaagagcctgtccccattttgtggggtcccagaaagggtgattttcacacttcacatttggcgttagggctagtatttcacaaacattaccgtctggaacttttgaaggctgagttaaatgacttcatcttgtgtacttgtagaactgctattagaaggaggctggggactccctgatgatgtgaaccagcccttctcttctcagtttcagcactttgtgatcatatttataaaccttggacagggctccttggccttagagttaagtgaagagttaacactgtgactttagatgcatttttctcagtgtaagtgtctgagctcatctccagttctggactcgtgcccccactcccgggtgctgacagcaagctctgaagatatgctgagagagctccctgactctcctgactcaggtctgccagttgatgaagacagcacagttgttaccaaaaacaaaacctggcagatggtcctaggtcaggattagtggtcattctctggtcttctccagactggtgacacagggtggtcttaaaagagtcctagtccagcctctggctccccgaatttcctctggtgcatggggcatcaggaaagggcaggggagaaggagggggagcagaaaaataactgaggctgggcagaagggtcccttccatttcccatccattgcttcaaacaatgggtgattaccgtgcaccttgagtgaatgtcaaggcaggaagggttccagggccacccagtgtggcaggagcagagctgggatcagaattctggattcctgactctccgttcagctgctattcagctatcaccaagctgctgagtttgtcctactttctcctgggtcacattctgctgttgttgggaaacagggagtgagtgaagggaacctgggaacccttctcccctcaccttttaaggatgttctggggtggaataaagagaggtggagggtggggtgcctgggcccagcctccctggctcctggtccttctttcttcagcagggagttctggcaaggcctgggcctggggagggggcctgggtgcagttttctcctgcggtgtggttggctcttttgtcttccaggatgggcctgtggggtctgtgtggggaaggaaggagctgggagcctgggctgcctgtgtgtcggctggatgctgccagcaccggcatggaccaagcaacttctctgagccagatgctggggacgcagaggtgaacccagagacatgatcccatctgctcagatcttcatgtcagaacacgctttattacagtctcggcaagggggtggctccatctgggccgcatctccaggactcctggcctggacttctctttggacctcgggtgaccttggattctggccacagtgacttagcggtcagactccggtggttttctagcccgctgggtgactccctgaagatctcatcctcttcacacctgttgggatggagcctcccatggataggaactcttcctggttgatgctcagcagatgactgatctggctctggaggacagctccaggggtatgagggaggcctggcagcccactactccctcctcacctgctcgggccctgcctgctgggccctattgtctgggtctgggggccctttcctgccccatgtgaacttgattcctcctcgcgtttctgccggtgaccagacagagcctggcctgggccaagtccccactgaccacactttccccaaggctgggtctcaaaggaccctacaggatggggacaggaacaaggatgggagtcggggaggggctgtcaggcagagggcagatgcagctcctcgggtgccgctggctggagtcctttgtctccatttgctcagtgcgtaattgtcattttgtgggggacttccttggtggggcttttccatgcttttcctctccctgatggttacaaagaggagacagtcaggatctgggaggagctggagccttccccctgtgctgttgaactctgggaggggcagccagaggggctgagagtgcaccatcctgaaaagagagggtctttaagagataagatttaggctagggaaattcactagaaagagacaggtggccctgggttttttttcctgccatccaaagggaaccagtcttacccaaaggcttaaggggtcaccttagcccagtctcaggacactcttctccaatcagatcgctgcttctagttgcactgctcctggattctgttcttggggaacagaaggtggcaggacaggccccacaatctgcaattccagctccctccagaggagacagtcccagcgtttgcaagagagcagcttgtggcctcccttaggcaagtttaaaagccagatgtcctttttcccagaatgcggagtggtgtgtactattcatcagcttgggctgccacagcaccatgccagactggatggcttaaacatcaggaatttattttctgccagttctggaggctggaaagtccagatcaaggtgttaagacttggtttctggtgaggcctctctccttggcttgcaggtggcaccttctcactatgtcctcacatggccttttctctgtggagagggacagagagcatgagcaggctctggtgtctcctcctcttcttataaagacactaatatcaccatattagggcttaaacctatgacctcatttaaccttaaccccttaaaggtcccatctccaaaaacagtcacatagcaggctactgcttcaacatatgcatttgggggaggggacaccattcagttcttaacagggtggtcaccgcaaacatggaaagtcagagccttctccccttcagaattcccgcccccacccagggatggggaggaggagcagagaggtatgggaagcagacacggagagtggcaggtaccatgctggggtggctcaggagtgcttcggaggacatatggaactggcagggctcagtgcagggaggcggaggccctgggagagccgtgtcctgagaagggcctgggctacaaccctgggcaagttacttcacctctgagcctccgatgctctgtgaaatggaaggaatgtgcttgcctgtcaggtgcagggagaattcagtgagattgtgtgcctagcacagtgcctgactcccagtaggtgctcagcaaatgctcccatccatctgggagtatagacttagggtttatctatttttttttttttttggctctctggactttaaaactcagcatcttctgaaccagaggcatttctgattagcccttccctacctattttcctagtatcactctttaatcagcttggggaggtggcagcatttcatggcctccgtagtaactcacaatgcttcctggggtatttaaattctactctctcatcagcactgagcacctactttgggccctttcccgtgctagccatttgggggaaatataggtgaagagtggctggggtttgaacctttaggatttcacagccatgctggatgggataaaaccagctcacatggagaatcagagaatgggacagtgccacaaaacagtctactgaggcacaagttctgagtgcctgggaagtggtagagagaccccagagagggctgctgcagtctggaacagcttcctagcaagctgtaccaaacagggccttgaaggatgagaaagatctggctgagatgatacccgaccctctagggaaattcttaaagtaacttctaggaaatgtcattgctccttaaaaaaaaaaaaaaaaaaaaaaaagcaggttctaattcaaattccgctgcactactgaagtagtgcaggacttatcagaacctttaaaatgctaatggcaatttcaaatactcagggacagggcataggatgaagtgttccttgacttttatggccgtagaatctttttctcatggaacatcgtttagaactgtgggagatgctaggctggggtttctatgggaaaagtacagcagtgatagaagatggcataattagggtacacttaggtgctttgtagcattggctgaaaaggaagcagaccgggtgggcgtgggggagtctcaacttggttccaggagccaggccagcttgatgcgttttttttttttctaatggtgggagggcaggccaatagttggagaaactagaggcctgacagtcggtgatggaagagatgtctactaagcacggttgtcagatcttttgcggagagatgcgggtgtgcgtgtgtctgtcagagtgtaacaggatccagccctctgggaaggggctgaccagaaccaggcagggggtttctgtctaggacaagtagggtccattttcaggggagctgaaatatttgcagggggtgctctgagacccacagccaataaatagggaagcaacatgcttttgggcagggtccatgtggttagggttgattttgatggggctggggggcttagagttagaccccctgccctggtcaggccccactccctggctggtgggggcgggaggcctctgaagctgcagctggaggggccggggcagtgttgcatggtgtgattaacctactaacccagggctcagccgggacgctgtggcgtgtgacgaggtgcctggccttctgccaaccaactaacccactaaccaggcccgagcatgccctactaaccacccccccgcagccagatcctactaactgtgcagctcatgggcctttcaaggggctgggatatgggggcagccaaggctccagaaggctctgcctatgggctgggatctttgtgcagacccacgttccaggacagagaatgcaaataagactcaccaactctgccacttcctgttgagtgagtttgagtaagtcacctcacttctctcaacctcagtttcctcagtggtagaatgcgagttctgataccttacaggttgcagtgaggattcccgtcaatataagggtctcagcacatggcagacacctaaaaaaaaaaaaagtatgaggatggaggaaggcccctcaaacctggttgaacacgtctgctgcctaggccctggcctctctgggtgttctgcacacagatccttctgcctggggcaagacacttggctttgaatccatgtcacttccccacattcttcctccgtctcagctttcttttctgtaggcagctcttttgttcgattactaacccgtctaacccctggggtgcctcaactgcaccccgatcatcccttaacaaactaacggacctgcgtgcgttcttccttttctcttagcgactcctgtgtgtgtctgctgaggtgccctgtccgctggtgctgtgctctgacttactaacccagcccctactaaccctgttttctcttcttactaaccccagccctgccgagctctgggctccccccgggggctggtccccctccttttggcaagcagatgacctggggctactggccctgtagacagatgtcccactttgctgccccatattggctgtaagatcagagtccactgggccaggtctaaggcaggggatggccctattaacaagactcagaggaggaagaggtggtcctgtggatgtgggaggctggactctgagtatgacatctctcctatgtgcagaagtctggttgccactgggagtaggtgggaccagggaaatctctgggacgtgagtgtggaggcctgttggtctagactctagactgtggagctctgagcttttgtgtcctctggaaggaagctggggaagaatcctctccattgttaagtgacggggatagaagctgtcctgcacaggaagtcacgaggggggcgtatcccacgaggaaggcaggagggggcgtgcccctcaccggaaattagcagaggggcgtgtcccacaccggaagtcagaaagcggagcctttcttacaccggaagtcaatgaagcgggtctttcctacgctaaaaaccactgagtggagtatttagtacacaggaagtcggccagagaaacatttctcatatttgaaggccggaaagagggacatttctgacaccggaagtcagtgagaggactctttcccacacaggaagtcagctagagagccgtctcccctctctggagccgagagaggccggtttcccccaccgtaagtagacgtggggccgtgaccggaagtccttgggaaagatccgtcccattcccggaagctagagggcgttagttgtcgggttgaaaaggggtgtggggaggggaagcagctttaccccgggctcggagtttgcaggagagagaagtggggagcaagaagtgaacctcaggggctcacagggttcccgcagatgctcaggccggccaggaatgcatctctggctctctgttcccacggacgtcactgcctcagccagcctcccccagagcccgccagccgctaagccggggccacacctgggggtgatttcatgcctcacctccagtaggcaccttggtttctttgggctaatctctggctcccttgcgctaactcttgctctcacccagctaatccctgcctcaccctgactgccccaggggctgaccactaacaaccaacctggccctgtctgggggttccaggctcctggcctggccctgaccggttcttaattaacctttccttcaccttgactaactcctgccttcctggtctgttcctttcagcagaaactaatggtttgtggatttttttctgactaacaacaggtctaacattcctcgttactgttaacagcttggatgtcggcatggctgggaaggggctaacacagctttgaacttggctaacacaggtttgaacttggctaacacaggtttgaacttgactaacacagggaaaagcatagctaacaattttgggcgtggtggctgctctgagtcagaacaatcagaagtcggtaaagatggtagttttctaaaggaggtgccagggctctggtgtggaccaggcctgatggagcagtggtacccaccaaggtggggtcagaagtatagccagtcttgcaaggttttggccattgggcatatcttcactcctcatagtctgcatttggtttcagttcttaaaaaaatatagccttatagctacagtagtttgcacaagtagatgcagctcttataaaccttaaaatacctgtctggtgtctacagagcatatgccattttgtatagagtcacccttccccaggccagggcctagagtcttcattttggggactttgtgttttggaagttctaggacataaagctttagatcagagtattcagaagggttataactccgtcagtcatttacatgttagtagtataaattatctgagcttcctgttctaacttttagctcttctagcataaactcttctgtgcaatttagctgcaccgaggaaacgggagtttttctggaagggacttttgatctctttagactgagggaacgtcctttgggagtagaggggcagggagcatacgcaagggattccaggtgcaggtaaaaggtggcactagttcaaggttttgctgactcagtctggtagtcagagtctgcaggagaagacagttcaaggcagggcctggaggattggatcagtttagggacaggtcaaaggctggcttacagaccttagaggcaggttgcttgggtcgttgaatgctagtctggtgctgagagcccttttctctggcaactgtggactcagagctaaccaattgtagttggcagtgggggtgaagggtgatccagaggcctgagctgcagagggcacaagagagaaaagatgtcttagaaagagctttgagaacatgccttggctgctggcagggaccttggatggggtagtctacacccggaagtgcctgcctgccatcctctagtggctgccttgctccatttcactcaaagcaggaagctcacacctcctattcctgaaactcctctttgtttaactgcaaagacttgatgctgctaaggatctactatgtgccaggcactgctctgggcgctgggacctgcacctgggctttttcgtcatggtgcttttatagcctagtgggagagttggtgaagtagatagtgattcagtgagatgggtgttatgattggtcaggggtctgtgggagcaccaaggagacagacaagattgatgtgcacctactctgtgccaggcgtgtgccaggcattggggatgtagtggtagttaaacaccatttggtcttcaggagctttaattctagtgtgttgggtgcaggggggtggaatggggacagagagacacctaatccaccctgtggtggctttctggagagggaggcatctaagctgagctgtggctgggtggagtgtgggtggggatgagttccgggcagcgagagtggtggacaccagtttctggggatcagagaggatccaaagaggttctggaaggttcatgtggaatgtagcaagagataggagacatggacatggtgccgggtctggttgccaagaagtttagattttatccttaggccttggggagcgacggatatgatctgagaaagggagttagtggatttgagttttaggctggccatttggcttttccagcccaggtggaactcagaggagtttgcaatggcctctggccacattttagacaactgagcagaactttttgaaactaggaagaccctttggtccatcttttgataaacagaatccatacatgtctaccccagttggaagtatctctgcaatgactggaaagtaaagaggaccaaggtgaaaataaaggctcggaaggggagcaatcttgaaaacatgtcatcccatggtggtgggaagtccctggagaagatcaggggaaacacagtcataggctgcaagtctataagataattccattggggagggagcccatttgtcatgcatggctgcaaggggcagatacaagtgtggagtaagcttgcaagagctgatcctggtcccagagagggaaaaatatgccttggtgggtaatgaaccttttgttcccagaggcagaaggattgggactaggccaacatagagattggcgatggttgtgagattctaagagtgtgtgtgcatcttgacaatattagaggaggctgagcccaagcaggcacattctcttcgacccctccctcattcagtctgctttggagtctactgaacatcaagcttgctatgagcaggatcttagagctgaggaattggcctcccaatccgaacaggtgttataatcctttcttaataggttgtgctgtggacccaatgtgagggctgtgctggtgtaaatggtgacatgttgagctggggggatgctttcggggtggggggactggttccattccatcaaaggccctcttgagagtctatccagggacccattgttttactttaacagaccagaaaagatgtttgttttccatgtcattacccccaggggataccgaatgtgtgggtagaaatttctctgtagattaaaaatcagatttttacatggattcaacaaaggagcgtcacttggatttttgttttcatccatgaatgtagctgcttctgtgtaaaatgccattttgctattaaaaatcaattcacgctggaaaaaaaaaaaaa

SEQ ID NO: 143 - DNA, 2190 bp, synthetic construct; identical to SEQ ID NO: 141
  source 1..2190: mol_type = other DNA

SEQ ID NO: 144 - DNA, 2308 bp, synthetic construct
  source 1..2308: mol_type = other DNA
ctagaatgagaatgcacagcaagcggcagacaatcacccggcattcggcatctctttcttttcacgcgctccctcgctccgcctttctccagctctgccttctcaggcagatacatcagataccttgtttatccatcttcagctccactctgagggcgcagacgcacgattccgggatcgggtgcaccacggcgaagcccaggcgggagacggcaggagcagctcagatgaagaaactgaaacccacagagatgaagtaacatcctcaagatcaaaaggtaacaaacgattgaggcaggatttggatattgctctacatgacgggaaagcctgtgctcttggaattacagtcatagtacaacagatcaagttcaaagcatcatctaagtcaaggaaaaccagtaaagaggtttggagagacaaacagggtcaactccaggcatttgggtcagcagaaacggcacgattgagcagcactgtgactataggatcatggatcagaggctgcttcctctttggttctgggcatcagcctcatgtccactcaaagtaagtggcccctctgattggaatcggaggtgcctgggtcatctcacagagccaaacaaatacaattagctattgcaaagccttttgggaattattcccagtgtaaataaacacataaccatatagcaagagccttgataaagtccaaaaacatgcaaacttggagtatctaagagaaaagaccacaatgtaaatgaaaaaccaaataaactcgggcaaaccataggatagggccctgtctgtgatggcctgcatatgatgagccatagaaaaaggatggtgaattctggataataagaaatgtcaatgagatgcaagaaccacctgttttatgtaaagctccaaataaccagatcacagtggacagccactcaaataatgccttcataatacagagtattattgagaataactcagttcacagagagcttaaggcagccaatatttgatagcctgtcagaaaaaaacagaacagtaattatagaaaagaatcatatcctcggaaaaacaaaaattaatcaaactaagtttgtaaagtctatcttacagacacattgtctggactggtcctctcaaaaatactgtttttttttaatgccaatttgtttagttaatgatttttgtcttattacttcaaaactggaaatatcctatgactcataatatcttacaacctttctactttcttaaagaatctcaagtttataatcacaggggatcggattatttttcaaaaattaaatggtgatgtaatgatttctgtgtctattgtagaaaagtcaaccttattacagctgcaacaatggcattaagaaatatgagtaattccaatcaacttgagataatgtctaatcaaacacaaatacaactggtaaatttcattaaatagcatggagattaaattaaaacactattatgtaataaaaacctttagtggtactaaaattttagaatagttcagatatacagaaaaatttcaaagatacacagagttcccatttttttcctattactaacctctcatatttgtcacaactaatgaatattcaatagagtattattaactaaagcctatacatttatttagatttccttagtttttagctaacattctttttctttgttccaggatcccatccgggccaccacattgaatttatttgtcattttaggtacctcttggctgtgagtttcttagactttccttgtttttggtgaccctgacagtttgagggagtactagtcagtcagttatttttgcagaatgccctaaatttgagtttggctgatgtttttcttagggtttgactggggttatgggttttggggaggaagaccacagaggtgaagtaccattctcaccaaattatattaaaggtacataccatcagcatgccttatactattgatgtgaactttgattgcctggctgtggtagtgtttgtcatgtttcttcactgtaaagttactcttctcatcacccacttttctgtactgtactctttggaagaaagtcactatatgcatcccaaatttaaggagtgggaagttatgctccacccatttgtaagcagaaaatctacataatttgtttggcattcttctgcataggaaaatcatctcactctcccagttatttatttatttgatctttttttatatcagtatggactcatgggtatttcttttatactttgggttataatccaatactaacacaataaagaaatttttaatggagatgcaaaaaaaaaaaaaa

SEQ ID NO: 145 - DNA, 8779 bp, synthetic construct
  source 1..8779: mol_type = other DNA
cgcagcctgcagcccgagacttctgtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggtattttaaaagttccgggggttttgtgaggtgtttgatgacccgtttaaaatatgatttccatgtttcttttgtctaaagtttgcagctcaaatctttccacacgctagtaatttaagtatttctgcatgtgtagtttgcattcaagttccataagctgttaagaaaaatctagaaaagtaaaactagaacctatttttaaccgaagaactactttttgcctccctcacaaaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaa

SEQ ID NO: 146 - DNA, 6983 bp, synthetic construct
  source 1..6983: mol_type = other DNA
caggcattcaggcagcgagagcagagcagcgtagagcagcacagctgagctcgtgaggcaggagactcagcccgaggaaatcgcagataagtttttaattaaaaagattgagcagtaaaaagaattagaactctaaacttaagctaatagagtagcttatcgaaatattacttagtcttaataatctaagaagatcttaagagataacatgaaggcttatttaaacagtttgaaaaaggaaatgaggagaaaagtatttgtactgtataatggaggctgaccagagcagtttaggagattgtaaagggaggttttgtgaagttctaaaaggttctagtttgaaggtcggccttgtagattaaaacgaaggttacctaaatagaatctaagtggcatttaaaacagtaaagttgtagagaatagtttgaaaatgaggtgtagttttaaaagattgagaaaagtaggttaagttgacggccgttataaaaatccttcgactggcgcatgtacgtttgaaggcatgagttggaaacagggaagatggaagtgttaggctagccgggcgatggtggcgcacgcctttaatcctagcacttgggaggcagaggcaggcggatttctgagttcgaggccagcctggtctacagagtgagttccaggacagccagggctacacagagaaaccctgtcttgaaaaaacaaaaaggttaggctagtatttggagaaagaagattagaaaatggaagtgaaagacgaagaagacatacaggaaggtgaagaaaaagctgttagagaagataggaaaatagaagacaaagcatctttagaagacagaaaaggtacttaaaggcacaggtagtaggaagccgaagaatagaagatagaaagaagcaagatagaaaaacaaaatggaagttaagacaactttggatgccagcattcaagataggcaaagaagataagattgaggccaaaaggttggataagatataaagtcagaaggaaattatctttaaagccataagttcaaatttctgatggagcgagcagtttagaagagtctttagacagccacatacaagattgaagctagcaatcaaagctactaggactgaagtaaaaagttaaggcagaatgcctttgaagagttagaagaatattaaaagccttaacttgtagcttaattttgcttgatgacaaaaggacttttgataacagtttcaagattgtcagcattttgcattggacttgagctgaggtgcttttaaaatcctaacgactagcattggcagctgacccaggtctacacagaagtgcattcagtgaactaggaagacaggagcggcagacaggagtcccgaagccagtttggtgaagctaggaaggactgaggagccagcagcagcagtgcatggtgaagatagcccaggaaagagtgcggttcggtggaggaagctaggaagaaggagccatacggatgtggtggtgaagctgggaaagggttccaggatggtggagcgagagcgagttggtgatgaagctagctggcggcttggcttgtcaactgcgcggaggaggcgagcaggcattgtggagaggatagatagcggctcctagaccagcatgccagtgtgcaagaaaggctgcagggagagcatgcggtgcggtaacattccttgaggtcggcaacatggtggtggttttctgtaacttggatggtaacttgtttactttgtcttaatagttatgggggagttgtaggcttctgtgtaaagagatatatctggggctgtatgtaggcctttgcgggtgttgtaggtttttctttttcagggttatgtcctcttgcatcttgtcagaagcttttgagggctgactgccaaggcccagaaagaagaatggtagatggcaagttgtctttaaccgctcagaggggaatgaatggtagagccagcacaacctcccagttttgtaagacgttgtagtttgaacagatgacctaccacaagcctcactcctgtgtaggggaggtaattgggcaaagtgcttttgggggaatgggggcaaaatatattttgagttcttttccccttaggtctgtctagaatcctaaaggcagatgactcaagggaaccagaaaaaaggaaatccactctcaggataagcagagctcgccaggtttacagtttgtaggaagtagaggatggatgctagctttcacactgagtgtggaggagctggccatggcggaattgctggtagtttactctttccccctcccttaatgagatttgtaaaatcctaaacacttttacttgaaatatttgggagtggtcttaacagggaggagtgggtgggggaaacgttttttttctaagattttccacagatgctatagttgtgttgacacactgggttagagaaggcgtgtactgctatgctgttggcacgacaccttcagggactggagctgccttttgtccttggaagagttttcccagttgccgctgaagtcagcacagtgcggctttggttcacagtcacctcaggagaacctcaggagcttggctaggccagaggttgaagttaagttttacagcaccgtgatttaaaatatttcattaaaggggaggggtaaaacttagttggctgtggccttgtgtttgggtgggtgggggtgttaggtaattgtttagtttatgatttcagataatcataccagagaacttaaatatttggaaaaacaggaaatctcagctttcaagttggcaagtaactcccaatccagtttttgcttcttttttcctttttctttttttgaggcgggcagctaaggaaggttggttcctctgccggtccctcgaaagcgtagggcttgggggttggtctggtccactgggatgatgtgatgctacagtggggactcttctgaagctgttggatgaatatagattgtagtgtgtggttctcttttgaaatttttttcaggtgacttaatgtatcttaataactactataggaacaaaggaagtggctttaatgaccctgaaggaatttcttctggtgatagcttttatattatcaagtaagagatactatctcagttttgtataagcaagtctttttcctagtgtaggagaaatgattttccttgtgactaaacaagatgtaaaggtatgctttttttcttcttgtgcattgtatacttgtgtttatttgtaacttataatttaagaattatgataattcagcctgaatgtcttttagagggtgggcttttgttgatgagggaggggaaacctttttttttctgtagacctttttcagataacaccatctgagtcataaccagcctggcagtgtgatgacgtagatgcagagggagcagctccttggtgaatgagtgataagtaaaggcagaaaaaataatgtcatgtctccatggggaatgagcatgagccagagattgttcctactgatgaaaagctgcatatgcaaaaatttaagcaaatgaaagcaaccagtataaagttatggcaatacctttaaaagttatggcttatctaccaagctttatccacaaaagtaaagaattgatgaaaaacagtgaagatcaaatgttcatctcaaaactgcttttacaaaagcagaatagaaatgaagtgaaaatgctgcattaagcctggagtaaaaagaagctgagcttgttgagatgagtgggatcgagcggctgcgaggcggtgcagtgtgccaatgtttcgtttgcctcagacaggtttctcttcataagcagaagagttgcttcattccatctcggagcaggaaacagcagactgctgttgacagataagtgtaacttggatctgcagtattgcatgttagggatagataagtgccttttttctctttttccaaaaagacctgtagagctgttgaatgtttgcagctggcccctcttaggcagttcagaattttgagtagttttcccatccagcctcttaaaaattcctaagccttgcaccgatgggctttcatgatgggatagctaataggcttttgcatcgtaaacttcaacacaaaagcctacatgattaatgcctactttaattacattgcttacaagattaaggaatctttatcttgaagaccccatgaaagggatcattatgtgctgaaaattagatgttcatattgctaaaatttaaatgtgctccaatgtacttgtgcttaaaatcattaaattatacaaattaataaaatacttcactagagaatgtatgtatttagaaggctgtctccttatttaaataaagtcttgtttgttgtctgtagttagtgtgggcaattttggggggatgttcttctctaatcttttcagaaacttgacttcgaacacttaagtggaccagatcaggatttgagccagaagaccgaaattaactttaaggcaggaaagacaaattttattctccatgcagtgatgagcatttaataattgcaggcctggcatagaggccgtctaactaaggactaagtaccttaggcaggtgggagatgatggtcagagtaaaaggtaactacatattttgtttccagaaagtcaggggtctaatttgaccatggctaaacatctagggtaagacacttttcccccacatttccaaatatgcatgttgagtttaaatgcttacgatcatctcatccactttagccttttgtcacctcacttgagccacgagtggggtcaggcatgtgggtttaaagagttttcctttgcagagcctcatttcatccttcatggagctgctcaggactttgcatataagcgcttgcctctgtcttctgttctgctagtgagtgtgtgatgtgagaccttgcagtgagtttgtttttcctggaatgtggagggagggggggatggggcttacttgttctagctttttttttacagaccacacagaatgcaggtgtcttgacttcaggtcatgtctgttctttggcaagtaatatgtgcagtactgttccaatctgctgctattagaatgcattgtgacgcgactggagtatgattaaagaaagttgtgtttccccaagtgtttggagtagtggttgttggaggaaaagccatgagtaacaggctgagtgttgaggaaatggctctctgcagctttaagtaacccgtgtttgtgattggagccgagtccctttgctgtgctgccttaggtaaatgtttttgttcatttctggtgaggggggttgggagcactgaagcctttagtctcttccagattcaacttaaaatctgacaagaaataaatcagacaagcaacattcttgaagaaattttaactggcaagtggaaatgttttgaacagttccgtggtctttagtgcattatctttgtgtaggtgttctctctcccctcccttggtcttaattcttacatgcaggaacattgacaacagcagacatctatctattcaaggggccagagaatccagacccagtaaggaaaaatagcccatttactttaaatcgataagtgaagcagacatgccattttcagtgtggggattgggaagccctagttctttcagatgtacttcagactgtagaaggagcttccagttgaattgaaattcaccagtggacaaaatgaggacaacaggtgaacgagccttttcttgtttaagattagctactggtaatctagtgttgaatcctctccagcttcatgctggagcagctagcatgtgatgtaatgttggccttggggtggaggggtgaggtgggcgctaagcctttttttaagatttttcaggtacccctcactaaaggcactgaaggcttaatgtaggacagcggagccttcctgtgtggcaagaatcaagcaagcagtattgtatcgagaccaaagtggtatcatggtcggttttgattagcagtggggactaccctaccgtaacaccttgttggaattgaagcatccaaagaaaatacttgagaggccctgggcttgttttaacatctggaaaaaaggctgtttttatagcagcggttaccagcccaaacctcaagttgtgcttgcaggggagggaaaagggggaaagcgggcaaccagtttccccagcttttccagaatcctgttacaaggtctccccacaagtgatttctctgccacatcgccaccatgggcctttggcctaatcacagacccttcacccctcaccttgatgcagccagtagctggatccttgaggtcacgttgcatatcggtttcaaggtaaccatggtgccaaggtcctgtgggttgcaccagaaaaggccatcaattttccccttgcctgtaatttaacattaaaaccatagctaagatgttttatacatagcacctatgcagagtaaacaaaccagtatgggtatagtatgtttgataccagtgctgggtgggaatgtaggaagtcggatgaaaagcaagcctttgtaggaagttgttggggtgggattgcaaaaattctctgctaagactttttcaggtggacataacagacttggccaagctagcatcttagtggaagcagattcgtcagtagggttgtaaaggtttttcttttcctgagaaaacaaccttttgttttctcaggttttgctttttggcctttccctagctttaaaaaaaaaaaagcaaaagacgctggtggctggcactcctggtttccaggacggggttcaagtccctgcggtgtctttgcttgactcttatatcatgaggccattacatttttcttggagggttctaaaggctctgggtatggtagctgatatcactggaacactccccagcctcagtgttgaactcttgataattaactgcattgtctttcaggttatgcccaattcgtcttattacctctgagtcgacacacctcctactatttattgaatactttgattttatgaaataaaaactaaatatctctca

SEQ ID NO: 147 - DNA, 2793 bp, synthetic construct
  source 1..2793: mol_type = other DNA
agctcagagatgcaaaaagcctgcggcgctctggaggcgcctggtcctggggacctctttcctccactctgttctcagcataggaagatggggctggggaattctttttggttttctggttttgttttgctctggttttggtcggggttttgtatttttctattcttccttggttttattttttcctttttttcctttttttttttcttttgctttcctgtcattctcccagccaaaagccgccttcgacccttttgatttgatgtagcaaagagtgtgtgtccagattatctgaaatagacactgaagaagctacatagatggtcaagcagggcccggaagaagaccattcaggaaaaagtggtccaggactcggtggatgggctgccggcagaccggtgtggctcagacctggtgactttttggagaggtcgtagaatgaggacagcagacacacgaggaccttgacttcggcgcacagacagtgcaaaaaggagacaaagacggacagaatcaaaaaagagcgtatttccccctcctacgtatttgtttttcctgatagtctcacaaagatgagagtctggtccggtccagcgggttatcttgtggcactggctgtctccagtggctgtgcctgcgctttgaggccactctgctgttcaccgtggcccaaaccagattcgggctgaaaggtcgctgagtcagacttttaggggctccgagaagcagcaagccctctgccttcactgttcctttttcttttatttttagctccccacccccacccctgctcccgctcgtgggctcttctcctcctggcccagggcgtgcgaaaaggtggatgaacctggaggcgggaggggccgggccaagggagacgggcaggaagcgggtgagtgcgaactgataacctcacatcagttgaggctgctgcatagcttcttggtgcgctgagggataccatgatgtatagctggggacgtaggagccagcgctgctgccggagcccttcccagatgaggagttcggattccagccgggtggtaccggtggggagccggcagacaaggctctgccgttcgccagcgcgctgccctccagagctgccccgccttgcttcatcagcttcttgaacttggagcgtttgttctggaaccatatcttgacctgcaggatcgggcgacaacgtgactggactatgaccattgagcctacccaggtccaaggagaaaagggacgggtggacaacaatacccgccctccttgctttgggaaagctgacagctccaggacagaggggcagagaccaccgcctccccacaccccatgagggccaaaggccagaaaattgagccccaggtccagccgctgttggcaacttttagagcagctatcagagccgggaataagagaacaggggagagccaggccttggcttgcaaatgagacgatgccacctgggcgacgtttatggaagacctcatgcagcacaatgcaataacaataaagaaaatttaaaacctagataggaacggggtggggggcgggggcatttgttaaaattagatctcctttcctcgtaggactattggaagaggctacagcgtctttgttaaactctttagactcaaaccaattctcccccccacccccaacggtgtatttgctttccccagctccaacctaaatctagagacagactggccctcgttcgaatgcctaggaactgcccagggtcttagagaagtgcaggaagaaaaaagaaaaatctcactttttttttcccccttcttcttaaggatctggaacttgggaagtgaagggccagagggaaggatctgcctctgccacctcgcaagaagaaggaggtgggggaagcaggcttaggcccaggacctgagtgattagtggcacactttaaaggctaaggaagattaaatcccccttctattgttccagttgttcaggcacgtgtgacctcagaacctccaaacaaaacgtctggggaaatccttccagttccctgcctggtcagaagaccaggtagcctgcaggtcagagagtagggtccgagctagggcaaacagtttgagggaggaagaggagacgctgaaagtcagaggtggatacacacacacacacacacacacacacacacacacgggttggggggcggactgtgacacctagaaccagaggcccagcgaacaaaaaatgtgaagcctggggctggagcccagctcccgaggcttgtttggcagagcggccaggtacctgcgtctgtgtgagtcccaaggaggccgccagctccgccctctcaggcagagctaagtactgagtttgttggaacctccggttcaaagcctgcaactgcaaactggaataaattgtcctgggtttacggatctttttccctttgccgttaaagcgcacctcgccgccttccaccaccgtactcttctcggagtccgcccctggagggacaacagaagcagagacccttgttaatgctgcactgcctttagtgaggagggacgagaatgaatgtctatggatgggttggcagaagtagggtccggaaaagaacgttcaagaaaccctgaaagactgcgaaggtccctcaaggtccgagcgcacgtccctggctgctgcagacagaattgggtcgttcccaagagcgcagctattgcctttgcagagaaagtttgctattttttgcaggcggtagttgaggtttaattacccttaattgcatacattgtttatagcgctacgcaataaataattaccgag

SEQ ID NO: 148 - DNA, 1000 bp, synthetic construct
  source 1..1000: mol_type = other DNA
ttttttttatctttgcaactcttaatctcgctacctccccctcctctcttctctcttctccctctctctcctcttttctctaccgctgtctctgagacttcttcttccctttttcctaaagaagttgatccagaaattcgaaaagccctgggcagagttgttgaatgggatgtgcaattagagaggggattttgttgggaggcaagagggtcccccaggctccacatagtccaggtcggcaggcaaggaaagacggctgaccagcccagcgggcgcggtttgcacatggtttgcacgtcgggccgcctctttctgcctataattcagctgcctctctgaggagtggtggaggagctgcgggagccgagaagcccaagagccctctggacccagaaaagtcctacagatgcccactcctcaccacacaacagaaggagctctggtcctgcctgccagccccagagggcactcaaactttggaggccagccacccggatgaagtgaataaggcctgaaaattgcttgtttgctcgtattgtaaaataataataattaccattattatttttaaactgtctaatttctctagggaaagtaacatcgaaagcctaaaacagacgccaaaaggcccatagaacacagagggccctctctgcctctggccaccacagcccctaggccaggcatgggtatttattcttaggtatgttgcttttaagaagctgtaatcagcatcttgagccgggcctccctttgtgaggcttctgtaactatggaagtgtgatttacgcagatttgtcggggtcagagacgtctttccctgagcactgtgtatatttagacaggactcggtttggtgttaaaaagtgtatatgttgaatggattcacacacagtagccaacaatgaccacattgtcggcccgtgtacaacgcgtattgaaacgcagcgcccagacttcaactaatctgccctcaataaagctgaaataattatcctaa

SEQ ID NO: 149 - DNA, 5352 bp, synthetic construct
  source 1..5352: mol_type = other DNA
aaaaccctcgccactcatgaagaactgaggcaaaaggatcacagatgctgctcggggccagccctatactccctccagtgcctggtctccggtggacccgcggggctctgcagttctctggcggccgcgccttgtaagcgctaagccgggaggagggcgcaggcagggcgcacagctctgagcgcgaccctacccggctcgggcccgcagcaaccccgactgtccgagagcctctccccaagcaaggctctgcgccgccctctgagcccacctcctggccacaacgctgcctgcgcttctccagcccaggccggaagccctatgctgcctcccgaagccggactttctggaagcccatcttcgtatgctcaccagcccaccctcctacgctggtcagagtgagattgctaacctcaagaccccattcaatgccagctttcaactactgctgaagaccaatgtaggtcctggaatagaagaactgcagagtatccggagctggccgaccccagactgattctcaacaggtggctgggtagtgccccccaccctgtgttggtcgtgcgggacccatcgcacacagagaagccaccttacccctcacctcaccccgtcggccaaagactgaccacccgcgcccgagtgtggggaggggtgggaattccggatcacacacacacacacacacacacacacacaccagggtaggggaacagggaagatcttacctgcagaaaggtaagagaagccgggagcgtgtagcagcggcggataaatattcattaggaagtgtaagtcctttcaaagggctccgcgcgcgcgctctagctcctgtctctctttctctccctccctctgtcctccaggaaggcttcccctcctctagctatctcaacgcccccccccccttcctgcgaaataccgggtggatcctaggctagctaacctgttgggaagcctgaactgcggtagcgcctctgcagctagcagtttgctttcgctcctggggcgcgcgtccccccccatcccctcccctccttcctttcgcccagcccctcacccctcctaaatcactacatcattagctttattgggcggggggggggcgggtaacggagcagccagacgcacctctccatagaagggtgaatagttggtgccctgcgatgcaaccccaactccatctccacctcattctcacccatctgcacatcctcatccaatcttgcttctttttcttcgctctttccacctccacctccatcctgtgtcaacctaactccctcagcagccctcccacccgccgccgccaatccggatcacagattgggttgggagtgagattgcaaggctgttaggtccgaaggtcctgtgtgtcgaggttagaactcctccctggcgtgagtacggggtggttatcaagatctccttctccttcctgaccctgcaaggttaccaaacagcctggacttttgaatcagatctctggagcaagcattaagaactccagccacacagatctcagggcctggccctttcactcagccgcggaggctgcttgagggatggggagaggtgatttcactcagcgccaacgcgcaggaaaaactcatagacgacagtcggactaatactcgtcccttaagaatgcactacctgaagttagtcgcaagtcatcttcaatcgaggaaaggaaaaaaaaaatcctcacatctagaagcttcgccgaagccacttccaacagcacgcgaacatgttcgcgcacacccttctctgtgcacgccccaaagcctgctctctcgcgggcaggtgaaaatacaggctgcccaggccagaggagagaaggcggcggaggaatacatcgcttagggttgttgagaaactaggaataactgttttcaatactctgctctgctaccaggaccttaggtccctcttgggaccctgaaaagtggctttgccccacgatggggggtgggggggcagtgactctagctctggccctcctactgggagcttaaagttggggtgaagggtttaaggaggagccgagggggcgggccctgttagtttgcagttgtaagtgcagctgtgcctgggacaccaagaaggagagtctcgccccctaatgctcacggtgcttgcttagaggacccccgaggttaactaccggagcttctgctctctcctgagccccaggccaacacaggacgatggtggccgggatgccagccgggatgcctaaacacagtaagacttcagttgcctctcatttggctgttcagagctaatgtctaccagaggaggttttagaagtgggtcgcacccgtcctgaatccatcctgtgaggtggcagagggtcagagggtcactccttcttgttaaaggttagcataataagtattattattactagggtagaggaagatgtggaggaaaaaaaaagcctagggtgcctaggacactaaccaaccttacagcttacccaaagtccagaacctcccattgtacaccttctctccccaacacagatccctaaggtctcctcttcatctggcatgtgtgttcacagaaggtacagtgtgcgagaacacacttaggaaattttaggaacgctaaccacagtactggaggaataaagatgtgtcgaaggaatggatggaatgcatgaacacatacaccttgttgtccagatgcagacaaaagctaccatgtccgcttggagtgacgcccagacacgcacagagatgtacaggaagccaaggagagtgggatccaccagagtacagtacaagtacagagtacagtgcaagatgcccagtggccctggagaaaaaccttcgttgactcttctgtatggaaagtttctgaagaaagggccaaggaatgatctgccccagaggctgcctttaggctcccatacctgctctgtttggagacacccagaagaggccagtacagcaaagactctttttggtttggttatttctgttgcttgttgtctttttttcatcctttgcctgggaaattgtgctctggaatagggcttgagtagacagcacaggcctccagtagataacaagagattctcaatctccaatgaggacattccgttctaaggaagcttccagataggggaaagccctgttttgtcccccttttctcctctgagatctgaaaaaatagaaagaaaaaaaaagaaaaacaaaacaaatataaaaactatagcccaatcccatgtacatcaaataaatccacaaggaatatgctagatcaagctaaattatgtatttaactcattttcttttgtgagatactgagaagtcttcaacttcctctattccaggcagggatcgaggacctggcctggcagaggttccaccaccagggtttcccacagtggttctgggactaggcgtaaggtgtcctgacttgttttctctgtcacctcttctggggaactggggaactgtcccagacaatggctgccccagttccctctagaagagacagcccccacccccagaacccgttgccctccaacctcacctcattttctcttgagctcattctccgagctcttaaagtagtaggtgtgagtcttggaccatttctttaaaagaaacaactaaggaaagggagggaggggaggagggaggaggggagcgggaaggagagctacctcctagactaacgcgtccaggaaccactctctaaggagcaaaagagagcctgtgttgtaaagtagaagtgatcccccgacattgctgcagggcgccttgcaacagatgtaactgtccactgaagagacagagggtgggcatggggaagattttttttcctcccttttgtgaacagctcttacacttttccaagacacattgagttcctggacatttttgtgaatttgttcttgattgtgttttgaacaataaactgtgaaaatcaacttcaagcccacacaatcagaatctttcttgggagggcaggttctttcagcgctgtttttctccaggtcgcttttatcttacccccaatgggagtcacctgctgatgctcctcgggaggagactgtaactgaaagacggaagtgaatgcagacacagcatcctctgagccaaaccgttgctttggttccttctagcttaggatgccgaacactggctggctttgaacaccctctacttcgctaagaagtttagagcctttctcacaacaaaccacagcgttttagaactggaaatgtttcttctgattttccagtcttaggacaattttcgaactctaacttaaaaaaaaatttgtttatgggggtttagctagaacacttgtagcattcgcttctcaaggaaagatcaactgttttctcataaccccaactgctccacacacttcataggatataatgtattttctttacttatccttgtgccttctgcccaggacatagaactaggggcaaaactttggaggtctatgacctcttgatagcacctgtaattgttaggcttttgttcttgtgccttgtaaatctctcatgtacgtctatgaaaatatgcattgcatgggtgtgatcctatcaagttgtccacactttctcaacaaccactgatgagtgaaaccatttctattgcccagggcaaagggctaatcgccttgtgggtctagagttgtcataggataatatccggtgtgtgtgtgatatgacagttattactttcaagaataaattaaaataaccagatacccctttaatactttcctcaacagcgtcaatgctctagagatgggacctcgccttccaaggtgtcttcactatcacagggcccgggctctctgcctcagcccaaccctgggcttgcgctcagagaggaggtattccgacggaaaaaaatcccatacatttttaactacagtgattactaaattaatagcaggcaccaagacagatgtcaaaacgtcttgaaaatgtttatctgtgaataattgagaaggtagtgggacgcattatctgattgtgtaatgaaatatgtatgaggaacagctgtttgcagcagccccttccccttgccgccttagccagggcccttctctcgcaagctttcttctactttcttccacctttttcttccttctactttctagttcgctctcttcttccttaactgctcgcacggctttggctgcagtgaacccagagtcgcctggttctctgatgaaggttagccacaaggtttcctctaagccttgctggattggcgttccgtagtgggatgaggcttcgttgtctgctgttctggccatcgcgccttccggccaggctgcccgctgggtctccatggagtctgcggg

SEQ ID NO: 150 - DNA, 1990 bp, synthetic construct
  source 1..1990: mol_type = other DNA
atttcacacctggatgtgctcactcaaccaagaatatagagaaagagcttctgccctgagactcagaaaaatattctcctgtgctttggttcagtatagatttctaaaccctgatcattgcttaagagatattcactgaggacagagtcttgctctattgcccaggctgcaactggtgtgatctcggctcactacaacctctgcctcctgggttcaagcgattctcctgcctcaagctcccaagtagctgggattacaagcatgcaccaccatgcctggctaatttttgtatttttagtggagacggggtttcgccacattggccagggtggtcttgaactcctgacctcaagtgatccacctgccttggcctcccaaagtgctgggattataagcatgagccactgcacccagccttatactgaactttcaatgggttcaattccactaggagcataaaggccactgcatatgagttgtggaaagaagagattagaagaaggaagaacttgagatgagttcctcccttcaacattctgtctcctcctacctagcatcttctttcttttagtctttctagaatgtccatctgtttttggccattgcggagagagaagctgagctttaaaggagtaggagcttcaaaggcgtaggagcttcaaaattcttgtttcttcatgtttgatcacccttctaaacctgtcttctgttccttctgctattcttttttcttagagcataggaaaggggagcttttaaattaatacttaaagcatggaaaaaaagaacttgagaagaaagtaaaacaagggagatgaggctagtaaagtaaggaaaatgaagaggaagaggaggaagggttagcttctaaattccaagtcaaattgatatggaacaggcaagccgcttgtcttacttaaacttcagaaaaggatctgctgaaacttgatagaaatggaaagggaaatccttggggtggggaacctccaaacattagtaatgatattgaacaactcaaagtattgaggaaatctgcaggctacatgcctgaagattacccatgcagatagaccaaaaggattagaattatctgttgatattagtaatatttattgacatctagctagtattggtaattttaagttttagattaatttctttggtaatagctatgatatattttatagacaagaattatatctataggcttgctatcataggctcttttaatcagcattaatttagtctactgatttttagcacatttgaatcattcacttatgctaggtaactcattgcaaaataaaaagatgattcctgtatgtatggcagctatacattaaggaggagtctaccagaatatgaaaaagtcagctgacctaaatattgctgagacaaaggaaaacccactcccttggaggagcatgaccttttcctgtaattcttcccactgctgttgttgagctccttggatcctggctcctggacaccatcatcaagaagactttatggatgggctgtccacccactgagagaagaggagcatcagctacagtttctctctagattgccttcttcattttgagtaatgactgtcagcagggtcagattaaacacaaaacaactggacaattgcttggaggactaaactataagggcactaacatgtcaatagtaggctaacacatccatggaaaatatatttaccagctcttctctcagggaggattctgtgtggggttggaagtaatgatttgttaaattccttaggggtagaaagtagggcataatcagaatatagaggaatatgctgtttgacttcagggtttctgtttttcttactaggatatataaaacagggactctagctagattgtttatgaccacagagggtaggctgagtgctcccatgatcttcctgcttggttcttgcccatacagaggtcagcctttcctctaataaagattgaacaagtagtggtctgaggga

SEQ ID NO: 151 - DNA, 4266 bp, synthetic construct
  source 1..4266: mol_type = other DNA
gcttcaaattggatggcactgcagctggaggctttgttcagaattgatcctggggagctacgaacccaaagtttcacagtaggaagggggaaaaaagaaaagaaaacatttttcctaatgtaacaatgcgaatgctagaaaatgacaagactgatcggttttaaaccattctgaagactgactgagcgtggaagttgctcaacaaaaaagggaacggggatattgaaccagagagaaacctacgcccagaagaacatgtccctggattgctttcccactgctgtggagtgtcttgaacactggtccctggacaccaacttcaagaagacttcatggatggctgtccagtcttatgagccacagtttcccctctacattttcttcactccagcgaggctcttatcagggtcagatcagagatgaaccagctggacgacagattggagcgctgacctcttagagtgctaacagtgaacagtgtggggtcagatctatagaaagataataataagaaaacaccctatatgcaagggagagggatggttcataatttcttaaagattgaaatcaaggaacaatcaaaatatagaagaatgtggacgtgttttgctgcaggacttctgttttgtccccattggaatatgtattatggtattcctgttggatcaggactcaggggcaaggctaagcattccagtggtcctcctacttagctcttgtcctttcgtaagaaacaccaactcattagtctctatattacttctctgtactgtagatctgcattcttgatctgagagatattggcaatgacactcttgtatgataaagctcaatgataagagtacttcaaacccccttgaactttttgtttatacatcaagtggtgacattgtgtattgagctaattagatcaatggagtcacagggtgatactgaactcttttaaaatatttggctgaaacatgacattgtagttatttgtagaagagaacattatggaatatgaaaaacatcacagaacacagaactagcagcagaaactagcagcaggtagacatttttccttttccatagagctttcaaccaaatgtctctgtagaaaatagtggctatcgtgtatatatatagccacatagatgtccttgagtgtaccctgtagtcagtgggagagttcctactgccacagtcatggccatggctatgttctctaagcctacattttataaacactctgtgaatcttgactacttttctttagcaagcattgcaaagtcctgggatgtcagagaagtgcctggggttggcagggtttctagagaggaaattgttaaatgatttgaaccagaaaacaaacaggggatggggttcagaaccaacaattacctctattctatgtaggaaaccacaacatgaaatatgctgggcatggaaactttgataccttggtttttcattctttttaaaaattaatactaaagagctatgcgactgtaggcaagccatttcccatcccctgtgaatatctcccagatgactttaaatcccttctagttctgaaaggcttttaacatcagggcccaggctccagtggccagtttcaaaataccctcccttttgatgttaggttacataaacattgttcttttttagggagggtctcttttatcaacttttaaaaacacacatcaggttctctggtattaaaaagatgccatctctgagtcccctactatctgtgctgcctgcctttcctcctgttctttccttattcccatccctattgaacttgtgctatgcagtatgcatcaggtatgtgttagctttggggatacatgatagataaactggacacacagggtcttcccattctcttctggaattttctttggagggagcctcttgtatctagacagaccgtgctgtggtaccccagaggtaaccacctacaggcttcactctgcctaagcaattttgctgtgcactaagatacacattcaagtaactttagattaccacaataactttctccaggtatgaggaaaagagataatttacttctgagatgtgtataggatagccctccatcctgggaagaacagtgactactccctgcatcccgaccttgcccagggaaagctaatgtttctctgtgttatccctgtgacttgccacttctttaaaaaggaatgggcaaacaataaacagacaaaaatgttgtctgacctcattggaaatccttttaagaattaatcctttctatctccttcattatcaacaaatctattgaatacttatctctgagtccagggcatattttataatacataaaacaatggaatttcaaaattggagcactgacatacaatattggttttgagtatttttattatagggaatgactttagacattgcaatttatgacttaactgataaaatggatgactcttgactttcaattttcattttcagttcagtcgaggaatagcttcctccaggtaatgtctatactttcctatgactaagggctctaactatctctgttgcttttctttatgtaggcatatgttagtatttattttctatatgacaaatgtattaaagaaagcatgaaattaatgagataaacttttcagataggagtttagaaaatcaaggggccaagataaataaatgaaaaatcaacttaaataattaacatattccagatatattggaataaatgtttattgtacccttttggttttgtcttgggttatttttttcttatctcactgatttttttttctttcctttttagcttttttgtcttttttgatttttgttgttgcgtttctcctttttttttttcttgttgatgttgtttgtttgtttgtttgtttgttttttgagaaagaacagaaggttggttggatagggaggtggggaagatctatctggatggagttgggaggagggaaaatacacgatcaaaatatattttgtgatgggcagggcatagtggtacatgtctttaatctcagcactctggaggcagaggcaggtggatctctatgagatggaggctagcctgatatacaaagtgagaccagaacatagggctgcctcaaaaactttatatatatattaaaaatgtttgctttttgagacagtcacagataaccaaaactgatcttgtaatgatgtaaacatgtccagctaattttcaaatattgtagggcagcatttctccctttgtgcacacgtggagtcagcaaatccatataattctaaccattctggtgaaaaggagaacactcggccaagcatctcacacttccaagtgtgaagccttgtttgaaagctccgagtatctaaatagtagccctgtgaaaggtaaatttatgaatggtctggtgtgttcttattccagccattgaccttaaagcaacttatatatgttttctttatccttcaagagaaaagaaaaatcatatttttccaagcaattaaaattcttctgcttcaggtaggaagaaggaattaggagttatgtctccttgtatataattgcaagtttcatttttcttgttttaatgattgacagaaaactgataaactgagacatctccttattagggttgaatgtactctcttggtggccccattgctaatttgtttgactattttccatgatttcttactctgtaatggaaaggtttattaaatatgagggttgcaaagctttctgaatactaatgaacttatttgccaaaatttaaatgttcttcttgtcagtgaatgcctgtctcacttaacaggcaccaaattgaataatgaagaaaattagactctatcgtaccctcaagagaaatcgcgtgtgaattgtaatagaaaattgagggagaaaagggtcatattgtagcaataacactagataatttggattattttaaaaaaggatgaacttagggaagctcaggtcttttcaaagaaacacacatttggttaattcatgcaaaacgctggtttcccctcaacccaggtggtctatacctatcgccagtttacagaaaaaggaagccaggtggatggaaaacgtgtgccaagtttctgtgcttacaatccactaaactcattctcatatgaggactttatatacctgtgatggagtgggaaaatcaataacctggaaaaaatgagtaccattttccaaagaagttcaataaagagatggaatttgggaaactgctgcagttcttcctataagcataaaaaaaaaaaaaaaaa

SEQ ID NO: 152 - DNA, 972 bp, synthetic construct
  source 1..972: mol_type = other DNA
attccggcgtggagttctctgagttcggtggcacttgggatattccacttcagtgccatgggaagaaaatggtaagcaaaaagcaaaaggaataatgcaaaaaaaaaaaaaaaattataatgttgtggtatccaattgctcctgcttcagccttttatccaaagccttgagatggatctgcctcatccaggaagttttcctgcttcgttagagtatcttcgttttctttatcagacgctggctggcactccacgccagtgcacggctccatggaatgcctgctgttggacacttgcagagctctgccaccaagcaacctcctgcattggaaatcgggggactactccaggatcttatgcgtgcttgctgtttgaagctgcagaaaagtgaataatcaattaagtattgatttcttgtaagatttggtaaatttcaaaggcaagacaaagaggatggcttaaggaaacactgcagggacttggcgtaaattagacaactaacggaagcgcagaataaaataacacctcaactgaagatttccttggaagaattagcactagctattggaagacaatggtggatttaatcaaaacagcaggcatgcaagaattttacccgtgaaccagaaaaaggagctaattataaccacctttttaatggagctaatttgactgaatggatgcaaatgtgctttatttatggatgaagaagcaagccttactgcatgatcttgggatcatcggatctgctaagctttgcagctcctgttaccttcagtacattttttatttccggagaaggtatttgtatgtaaacaacaactaaccatgcaaaagattcatcggaacaatatactgtggtatatgagatataatcataattttgcagcctctcaggatttcctctaacttggaggttttgcaacctttgtgtgcaatgggttatggtaaacaataaatgaatgatgctaaa

SEQ ID NO: 153 - DNA, 3129 bp, synthetic construct
  source 1..3129: mol_type = other DNA
ggggagccgccggagggcagctggggctgcggaattttggaagcgcagaagttttctcctcctgtcctgtaactggttttcactcactcaactgagagatttctttgttgcaatgatggggtctaagataacacttctggaggctgcaggcgagaaacacagctgatatcatcttttattgtgtgttgttatttgcctagcattataaattaaggaggaatagtaacaaagagcttgaagacatgcacagctcacaggccccgggtggaggctggcgacatcagacagacagaaccaagacatctgaggggcaaccaggaggtgcgtgtggctgcagagcacacagacttgtctttggacaaaattgagaagaactcataaacttggcttcctcgcttctgctcctcagacgcgcgtggatgatttgaagatattagcctaacaagatggaagcaggtttaaaattttacataagaagttacctttttcagttgtgttgatgttcctttcagctggtcctatagtacccctcctcaggaatgtctccccagtgcaaggacaaagactgaagagactgctatattgatggactctcaagccaactatgaagttgaaacaaagaaagtgatcacctgaagacacctcctctgctaagaaacacccccaaattgtgcagcttctgccactagaactctcagaacaagagacaatcttttcaagaaacagaaaaactcaataatgacatctagattttcatgagccaagaactttcccttcctcatgtgtattcctctgtttgtacttaaattcatgtgacattcatttttttcctagtatggatatgcttattaatgcacttgtttcaaaatcccaaattgcacaaatgtgttaatattttaagaaacaaaatgaatcctacaaggagaatgatttttagccacacatagggttggatcttgagagtgacctacagaataaaagtacttttaaaataaagtagtcagaggctattcaaagggtaaaataatcatagtaccacattggtccacttgacactaaccaatcgatcatttttttttaatcaagaaagctagattctatcagataaaatcactgcttctaaagagtttaaatctagttagaaaaagttatagaaatgtttgcaaagataagtaacagatagagtcagtagaggataagatcaaaaacaaaaccaagcaaaagatgagttcaggggagtttgccatcaagttggcaaaactgacttacttagggaagaaagttataaaacaggaaaatatgagatgaaccttgagtgatgtggaagatttagataaatggaaaggaaggagaaaatggagttctttaggtggttgtaattggaggaggaaatgaatacacacatcttgttgacttaaacccagacattcagcagctctctatacatatctggaaaagactgcacagtcacctcctgtctctcaccccaggtattacttagaattattatcatatttcccttcctttaaagtaagtaagggtgattggtgacaatatggagaactatgatttttccattaacctaataataattggtatttattgagttctgttaagcattttacatattaactcacttaagcctttcaacagccttgcaaaataggtattattatccccattttacaggcaagaaaactgaggtttaagtaacttgccgaagtgccatatacagggctcacattcagtattgcagttgcaaagctcatgatctatagtgccaagttgcaatattgtagtcaatgtcacaattattacccctttttatattccttgatatttttccatggcaaacaattagctatttcatttaataatcacctaaaacttttcagtcttctgattaaaattacgctggagtgatagaatgtattttcatgatagaaattgggaaaaaaaatggggaatgaagtttatcagcatttcagacttgttttttttttttttttttgcaagactttgatgagattgttcacttttgtctatgtaaaatcccaaatccttgagaataaaaaagggggaggtttaagtcacttgttgcaatgccctttttaatagaggcaataaatctaaaggccataaatttagagtgacttacagaagatcgaactttggagtgtggcagagtaagggatggaaaccgggccctccagttcactatcagtagcttttgcactggtctgcccttcctaaattaagtatgcacttcaatttgatgagtggaaacagtctatctgggcagtaaccagggagctttgtgcctagtagattgcttctgttctgcacttctttggtttcccacctcaatgtaaaaaatagctagcaatgaagtccagaagttgtcaatggttcatccccagaagaatgcataatgtccaaagttgtatgtgtatgatgtcttcaatggtattaagttatttcaaattcttagttcacctacataaatcatttctaacaagcatcttcttaaccaactttatgcacagtgtatgtttgtaagtgcttctgcacgaatgtttatacatgactgtttccatagtacttatgtttttaaaaatattcagtcatttcctactataatcctcatgtatccatgtaactgactcaaaaatacttcagccacagaaagctaaaactgagcaaatctcattcttcttttccatcccctttgcatgtggctggcatttagtaatgattaataatatggccagctgaataacagaggtttgagacacaattctttctcaaaggagtcagctaagctgggtctacttatggacaaacatctaaatgtgtggaagtatctgatatttgacaatggtaaatttccacttagctagctagcattgtcagacttcaatctcctcatggctctggccgtcctgttttaagcatgataattgttggccacatctcacatagttctcattgagtgagttcataaataaacagggtttttttttttttttaaagagcagccaagcacaaagtgtgactttgttgacattttatgtgactttgtcatatgttcctaacccccaataaaagcaatgttgcatcaactgtgaa